SEQUENCE LISTING (2 sequences)

SEQ ID NO: 1 - protein, 175 aa, Homo sapiens
  MOD_RES (146)..(146): Modified with acrylamide
MLPPMALPSVSWMLLSCLMLLSQVQGEEPQRELPSARIRCPKGSKAYGSHCYALFLSPKSWTDADLACQKRPSGNLVSVLSGAEGSFVSSLVKSIGNSYSYVWIGLHDPTQGTEPNGEGWEWSSSDVMNYFAWERNPSTISSPGHCASLSRSTAFLRWKDYNCNVRLPYVCKFTD

SEQ ID NO: 2 - protein, 16 aa, Homo sapiens
  MOD_RES (11)..(11): Modified with acrylamide
NPSTISSPGHCASLSR

(19) United States
(12) Patent Application Publication
(10) Pub. No.: US 2013/0115232 A1
(43) Pub. Date: May 9, 2013

(54) Methods for detecting graft-versus-host disease

(71) Applicants: The Regents Of The University Of Michigan, Ann Arbor, MI (US); Fred Hutchinson Cancer Research Center, Seattle, WA (US)

(72) Inventors: James Ferrara, Ann Arbor, MI (US); Sophie Paczesny, Ann Arbor, MI (US); Samir M. Hanash, Mercer Island, WA (US); Thomas Braun, Ann Arbor, MI (US); John Levine, Ann Arbor, MI (US)

(73) Assignees: FRED HUTCHINSON CANCER RESEARCH CENTER, Seattle, WA (US); The Regents Of The University Of Michigan, Ann Arbor, MI (US)

(21) Appl. No.: 13/573,766

(22) Filed: Oct. 3, 2012

Related U.S. Application Data
(60) Provisional application No. 61/542,630, filed on Oct. 3, 2011.

(51) Int. Cl. A61K 39/00; G01N 33/53

(52) U.S. Cl. 424/184.1; 435/7.92; 435/287.2; 514/169

(57) ABSTRACT

The disclosure relates to the development of methods for detecting or predicting graft-versus-host disease (GVHD) and for detecting or predicting response to treatment for GVHD. More particularly, the disclosure provides new biomarkers and combinations of biomarkers for detecting or predicting gastrointestinal GI GVHD and for predicting and analyzing response to treatment for acute GVHD.

### RELATED APPLICATIONS

This application claims benefit of U.S. Provisional Patent Application Ser. No. 61/542,630, filed Oct. 3, 2011, which is incorporated herein by reference in its entirety.

### GOVERNMENTAL RIGHTS

This invention was made with government support under grant numbers HL101102, CA039542, and HL007622 awarded by the National Institutes of Health. The government has certain rights in the invention.

### INCORPORATION BY REFERENCE OF MATERIAL SUBMITTED ELECTRONICALLY

Incorporated by reference in its entirety is a computer-readable sequence listing submitted concurrently herewith and identified as follows: 2,188 bytes ASCII (Text) file named “46007A_SeqListing.txt,” created on Oct. 3, 2012.

### FIELD

The disclosure generally relates to methods for detecting graft-versus-host disease (GVHD). In some aspects, the disclosure provides biomarkers associated with acute GVHD and predicting outcome in subjects with acute GVHD. In additional aspects, the disclosure provides biomarkers associated with gastrointestinal (GI) GVHD and methods of using the biomarkers to detect and predict GI GVHD.

### BACKGROUND

Graft-versus-host disease (GVHD) is a common complication of allogeneic bone marrow transplantation in which functional immune cells in the transplanted marrow recognize the recipient as “foreign” and mount an immunologic attack. It can also take place in a blood transfusion under certain circumstances.

Acute GVHD, a leading cause of non-relapse mortality (NRM) after allogeneic hematopoietic cell transplantation (HCT), is measured by dysfunction in three organ systems: the skin, liver and gastrointestinal (GI) tract (Cutler et al., Manifestation and Treatment of Acute Graft-Versus-Host-Disease, Appelbaum et al., eds., Thomas' Hematopoietic Cell Transplantation, 4th edn. Oxford: Blackwell Publishing Ltd; 2009. p. 1287-303; Mowat et al., Intestinal Graft-vs.-Host Disease, Ferrara et al., eds., Graft-vs-Host Disease, 3rd edn. New York: Marcel Dekker; 2004. p. 279-327; Ferrara et al., Lancet 373: 1550-61, 2009). Acute GVHD of the GI tract affects up to 60% of patients receiving allogeneic HCT (Martin et al., Biol. Blood Marrow Transpl. 10: 320-7, 2004; MacMillan et al., Biol. Blood Marrow Transpl. 8: 387-94, 2002). This dysfunction manifests with nausea, vomiting, anorexia, secretory diarrhea and, in more severe cases, abdominal pain and/or hemorrhage. Thus, the etiology of diarrhea following HCT presents a common diagnostic dilemma.

Acute GVHD typically occurs between two and eight weeks after transplant, but may occur later, and is often clinically indistinguishable from other causes of GI dysfunction such as conditioning regimen toxicity, infection or medication. Endoscopic biopsy is often used to confirm, the diagnosis, but histologic severity on biopsy has not consistently correlated with clinical outcome. Clinical stage two or greater (more than one liter of diarrhea per day) is associated with reduced survival, but daily stool volume can vary considerably. Lower GI GVHD responds poorly to treatment compared to other target organs, and treatment with high-dose systemic steroid therapy carries significant risks, especially infectious complications in profoundly immunosuppressed patients.

The art to date does not disclose methods for non-invasive diagnosis of GI GVHD. Accordingly, a strong need in the art exists for a non-invasive, reliable biomarker specific for GVHD of the GI tract that would significantly aid in the diagnosis and management of patients with this disorder. The following disclosure describes the specifics of such a biomarker.

### SUMMARY

The methods described herein were developed to provide a means for detecting or predicting GVHD, and in some aspects, predicting outcome in the treatment of GVHD.

In some aspects of the disclosure, methods are provided for detecting or predicting GI GVHD by measuring elevated levels of regenerating islet-derived 3-alpha (REG3α) or ST2 in a biological sample from a subject compared to a control level. Thereby, the disclosure provides methods for earlier treatment of GI GVHD in a patient at risk of developing GI GVHD.

In other aspects of the disclosure, methods are provided for predicting outcome of acute GVHD at symptom onset. The identification of steroid-refractory GVHD biomarker panels at symptom onset has tremendous potential for impacting the ability to risk stratify patients before initiating GVHD treatment. It may also ultimately guide the intensity and duration of treatment and minimize the toxicity associated with chronic steroid administration. The ability to identify patients who will not respond to traditional treatment and who are at particularly high risk for morbidity and mortality could permit tailored treatment plans, such as additional immunosuppressive treatments for high-risk patients that may be more effective if introduced early. Equally important is the identification of low-risk patients who will respond well to treatment. These patients may tolerate a more rapid tapering of steroid regimens to reduce long-term toxicity, infections, and a loss of the graft versus leukemia effect. Follow-up marker monitoring in high-risk patients could also help decide whether to taper the treatment.

In some aspects, the disclosure includes a method for detecting GVHD in a subject, the method comprising measuring a level of a biomarker in a biological sample isolated from the subject, wherein the biomarker is regenerating islet-derived 3-alpha (REG3α), and wherein an increased level of the biomarker present in the biological sample compared to a control level indicates GVHD in the subject.

In some aspects, the disclosure includes a method for treating GVHD in a subject suffering from GVHD, the method comprising the steps of identifying the subject at risk of suffering from GVHD, measuring a level of a biomarker in a biological sample isolated from the subject, wherein the biomarker is REG3α, and wherein an increased level of the biomarker present in the biological sample compared to a control level indicates GVHD in the subject, and administering an effective amount of a treatment for GVHD to the subject.

In some aspects, the disclosure includes a method for determining efficacy of a treatment for GVHD in a subject suffering from GVHD, the method comprising the steps of administering to the subject the treatment for GVHD, and measuring a level of biomarker in a biological sample obtained from the subject, wherein the biomarker REG3α, and wherein a decrease in the level of the biomarker after treatment compared to the level of the biomarker before the administration of the treatment indicates that the treatment is effective for treating GVHD in the subject.

In some aspects, such methods further comprise measuring a level of a second biomarker or a combination of biomarkers selected from the group consisting of: interleukin 2 receptor alpha (IL2Rα), tumor necrosis factor receptor superfamily member 1A (TNFRSF1A or TNFR1), interleukin 8 (IL-8), hepatocyte growth factor (HGF), and elafin in a biological sample, and wherein an increased level of the biomarker present in the biological sample compared to a control level indicates GVHD in the subject.

In some aspects, such methods further comprise measuring a level of a second biomarker or a combination of biomarkers selected from the group consisting of: IL2Rα, TNFR1, IL-8, HGF, and elafin in a biological sample, and wherein a decreased level of the biomarker present in the biological sample compared to a control level indicates that the treatment is effective for treating GVHD in the subject.

In some aspects, the disclosure includes a method for predicting GVHD in a subject, the method comprising measuring biomarker level for a combination of biomarkers in a biological sample isolated from the subject, wherein the combination of biomarkers comprises REG3α, IL2Rα, and elafin, and wherein an increased level of each of the biomarkers in the combination of biomarkers present in the biological sample compared to a control level of each biomarker predicts GVHD in the subject.

In some aspects, such increased level of the biomarker is more than about 25% the control level. In some aspects, such increased level of the biomarker is more than about 50% the control level. In some aspects, such increased level of the biomarker is more than about 100% the control level. In some aspects, such increased level of the biomarker is more than about 200% the control level. In some aspects, such increased level of the biomarker is more than about 500% the control level.

In some aspects of the disclosure, such increased level of the biomarker is more than about two times the control level. In some aspects, such increased level of the biomarker is more than about five times the control level. In some aspects, such increased level of the biomarker is about 10 ng/ml. In some aspects, such increased level of the biomarker is about 25 ng/ml. In some aspects, such increased level of the biomarker is about 50 ng/ml. In some aspects, such increased level of the biomarker is about 100 ng/ml. In some aspects, such increased level of the biomarker is about 150 ng/ml. In some aspects, such increased level of the biomarker is about 200 ng/ml.

In some aspects, the level of the biomarker after treatment is at least or about 25% less than the level of the biomarker prior to administration of the treatment. In some aspects, the level of the biomarker after treatment is at least or about 50% less than the level of the biomarker prior to administration of the treatment. In some aspects, the level of the biomarker after treatment is at least or about 75% less than the level of the biomarker prior to administration of the treatment.

In some aspects, the disclosure includes a method for predicting a subject's response to a treatment for GVHD, the method comprising measuring a level of a biomarker in a biological sample isolated from the subject, wherein the biomarker is ST2, and wherein an increased level of the biomarker present in the biological sample compared to a control level predicts lack of effectiveness of the treatment for GVHD in the subject.

In some aspects, the disclosure includes a method for detecting effectiveness of a treatment for GVHD in a subject undergoing treatment for GVHD, the method comprising measuring a level of a biomarker in a biological sample isolated from the subject, wherein the biomarker is ST2, and wherein an increased level of the biomarker present in the biological sample compared to a control level indicates lack of effectiveness of the treatment for GVHD in the subject.

In some aspects, the disclosure includes a method for detecting GVHD in a subject, the method comprising measuring a level of a biomarker in a biological sample isolated from the subject, wherein the biomarker is ST2, and wherein an increased level of the biomarker present in the biological sample compared to a control level indicates GVHD in the subject.

In some aspects, the disclosure includes a method for treating GVHD in a subject suffering from GVHD, the method comprising the steps of: identifying the subject at risk of suffering from GVHD, measuring a level of a biomarker in a biological sample isolated from the subject, wherein the biomarker is ST2, and wherein an increased level of the biomarker present in the biological sample compared to a control level indicates GVHD in the subject, and administering an effective amount of a treatment for GVHD to the subject.

In some aspects, the disclosure includes a method for determining efficacy of a treatment for GVHD in a subject suffering from GVHD, the method comprising the steps of: administering to the subject the treatment for GVHD, and measuring a level of biomarker in a biological sample obtained from the subject, wherein the biomarker is ST2, and wherein a decrease in the level of the biomarker relative to the level of the biomarker prior to administration of the treatment, indicates that the treatment is effective for treating GVHD in the subject.

In some aspects of the disclosure, the ST2 level in the subject is about 50% greater than the median control level. In some aspects, the ST2 level in the subject is more than about 25%, more than about 50%, or more than about 75% the control level. In some aspects, the level of ST2 is at least about 200 pg/ml.

In exemplary aspects of the disclosure, a high or increased level of ST2 at therapy initiation is defined as an ST2 concentration of greater than about 740 pg/mL, and a low or decreased level of ST2 at therapy initiation is defined as an ST2 concentration at therapy initiation of less than or equal to about 740 pg/mL. At about D14 post-HCT, a high or increased level of ST2 is defined as an ST2 concentration of greater than about 600±200 pg/mL for patients who received chemotherapy-based full intensity conditioning, of greater than about 300±100 pg/mL for patients who received reduced intensity conditioning, and of greater than about 1660±500 pg/mL for patients who received total body irradiation-based full intensity conditioning.

In some aspects, the disclosure includes a method for treating GVHD in a subject at risk of suffering from GVHD, the method comprising the steps of identifying the subject at risk of suffering from GVHD by measuring a level of a biomarker or a combination of biomarkers in a biological sample isolated from the subject, wherein the biomarker is REG3α or ST2, and wherein an increased level of the biomarker present in the biological sample compared to a control level indicates GVHD or risk of GVHD in the subject, and administering an effective amount of a treatment for GVHD to the subject at risk of suffering from GVHD.

In some aspects, the disclosure includes a method of determining susceptibility of developing GVHD in a subject, the method comprising: analyzing a biological sample from the subject to obtain level of a biomarker or a combination of biomarkers in a subject, wherein the biomarker or the combination of biomarkers is selected from the group consisting of REG3α, ST2, and REG3α and ST2; and assessing a clinical parameter or a combination of clinical parameters in the subject, wherein the presence of an elevated level of the biomarker or combination of biomarkers and the presence of a clinical parameter or a combination of clinical parameters associated with increased risk of GVHD indicates that the subject is susceptible of developing GVHD.

In some aspects, the disclosure includes a method of determining susceptibility of developing GVHD in a subject, the method comprising: analyzing a biological sample from the subject to obtain level of a biomarker or a combination of biomarkers in a subject, wherein the biomarker or the combination of biomarkers is selected from the group consisting of REG3α, ST2, and REG3α and ST2; and calculating a risk score or probability as an indicator of the subject's susceptibility of developing GVHD based upon level of the biomarker or the combination of biomarkers.

In some aspects, the disclosure includes a method of determining susceptibility of developing GVHD in a subject, the method comprising: analyzing a biological sample from the subject to obtain level of a biomarker or a combination of biomarkers in a subject, wherein the biomarker or the combination of biomarkers is selected from the group consisting of REG3α, ST2, and REG3α and ST2; assessing a clinical parameter or a combination of clinical parameters in the subject; and calculating a risk score or probability as an indicator of the subject's susceptibility of developing GVHD based upon level of the biomarker or the combination of biomarkers and the clinical parameter or the combination of clinical parameters.

In some aspects, the biomarker or the combination of biomarkers further comprises a biomarker or combination of biomarkers selected from the group consisting of elafin, TNFR1, IL2Rα, IL-8, and HGF.

In some aspects, the clinical parameter or the combination of clinical parameters comprises any of the clinical parameters selected from the group consisting of: age of the subject; whether the subject received a bone marrow transplantation or a peripheral blood stem cell transplantation, whether all human leukocyte antigens were matched or mismatched in the transplant, whether subject received previous treatment with tacrolimus and methotrexate, whether subject received high toxicity conditioning without total body irradiation; and whether subject received high toxicity conditioning with or without total body irradiation.

In some aspects, the combination of biomarkers comprises REG3α, elafin, TNFR1, and IL2Rα. In some aspects, the biomarker is REG3α. In some aspects, the biomarker is ST2.

In some aspects, the methods of the disclosure further comprise a step of administering a treatment for GVHD after determining that the subject is susceptible or at risk of developing GVHD. In some aspects, the treatment for GVHD comprises administering a steroid, administering an immunosuppressive drug, or administering a combination of steroid and immunosuppressive drug.

In some aspects, a biological sample of the disclosure is collected from the subject at about day 5 to about day 10 after transplant. In some aspects, the biological sample is collected from the subject at about day 7 after transplant.

In some aspects, the methods of the disclosure involve determining a risk or probability of developing GVHD. In exemplary aspects, a probability of about 0.33 or greater in a subject having received an unrelated donor transplant is indicative of the subject being susceptible or at risk of developing GVHD. In further exemplary aspects, a probability of about 0.38 or greater in a subject having received a related donor transplant is indicative of the subject being at risk of developing GVHD.

In some aspects, the disclosure includes a system for identifying susceptibility of developing GVHD in a subject, the system comprising: at least one processor; at least one computer-readable medium; a susceptibility database operatively coupled to a computer-readable medium of the system and containing population information correlating level of a biomarker or a combination of biomarkers in a subject to susceptibility to developing GVHD in a population of humans, wherein the biomarker or the combination of biomarkers is selected from the group consisting of REG3α, ST2, and REG3α and ST2; a measurement tool that receives an input about the subject and generates information from the input about the level of the biomarker or the combination of biomarkers in the subject, wherein an elevated level of the biomarker or the combination of biomarkers is associated with increased susceptibility to GVHD; and an analysis tool that is operatively coupled to the susceptibility database and the measurement tool is stored on a computer-readable medium of the system, is adapted to be executed on a processor of the system, to compare the information about the subject with the population information in the susceptibility database and generate a conclusion with respect to susceptibility of developing GVHD for the subject. In some aspects, the susceptibility database further comprises population information correlating a clinical parameter or a combination of clinical parameters in the subject to susceptibility to developing GVHD in a population of humans to susceptibility to developing GVHD in a population of humans; and wherein the measurement tool further generates information from the input about the clinical parameter or combination of clinical parameters in the subject, and the impact of the presence or absence of the clinical parameter or combination of clinical parameters on identifying susceptibility of developing GVHD. In some aspects, the clinical parameter or the combination of clinical parameters comprises any of the clinical parameters selected from the group consisting of: age of the subject; whether the subject received a bone marrow transplantation or a peripheral blood stem cell transplantation, whether all human leukocyte antigens were matched or mismatched in the transplant, whether subject received previous treatment with tacrolimus and methotrexate, whether subject received high toxicity conditioning without total body irradiation; and whether subject received high toxicity conditioning with or without total body irradiation.

In some aspects, a system of the disclosure further includes a communication tool operatively coupled to the analysis tool, stored on a computer-readable median of the system and adapted to be executed, on a processor of the system to communicate to the subject, or to a medical practitioner for the subject, the conclusion with respect to susceptibility to GVHD for the subject. In some aspects, the measurement tool comprises a tool stored on a computer-readable medium of the system and adapted to be executed by a processor of the system to receive a data input about a subject and determine information about the level of the biomarker or the combination of biomarkers in the subject or a clinical parameter or a combination of clinical parameters of the subject from the data.

In some aspects, the data is biomarker level information, and the measurement tool comprises a protein or nucleic acid analysis tool stored on a computer readable medium of the system and adapted to be executed by a processor of the system to determine the level of the biomarker or the combination of biomarkers from the biomarker level information.

In some aspects, the input about the subject is a biological sample from the subject, and wherein the measurement tool comprises a tool to determine the level of the biomarker or the combination of biomarkers in the biological sample, thereby generating information about the level of the biomarker or the combination of biomarkers in the subject.

In some aspects, the measurement tool includes: an immunoassay containing an antibody or a plurality of antibodies attached to a solid support; a detector for measuring interaction between a biomarker protein or combination of biomarker proteins from the biological sample and the antibody or the plurality of antibodies to generate detection data; and an analysis tool stored on a computer-readable medium of the system and adapted to be executed on a processor of the system, to determine the biomarker protein level(s) based on the detection data.

In some aspects, the communication tool is operatively connected to the analysis tool and comprises a routine stored on a computer-readable medium of the system and adapted to be executed on a processor of the system, to: generate a communication containing the conclusion; and transmit the communication to the subject or the medical practitioner, or enable the subject or medical practitioner to access the communication.

In some aspects, the biomarker or the combination of biomarkers further comprises a biomarker or combination of biomarkers selected from the group consisting of elafin, TNFR1, IL2Rα, IL-8, and HGF. In some aspects, the biomarker or the combination of biomarkers is selected from the group consisting of REG3α, elafin, TNFR1, and IL2Rα. In some aspects, the biomarker is REG3α. In some aspects, the biomarker is ST2.

In some aspects, the communication expresses the susceptibility to GVHD in terms of a risk score, or probability of developing GVHD.

In some aspects, the analysis tool further generates a treatment regimen to the medical practitioner based upon the risk score, or probability of developing GVHD. In some aspects, the treatment regimen is a more aggressive therapy if the subject has a high probability of developing GVHD. In some aspects, the treatment regimen is a less aggressive therapy if the subject has a low probability of developing GVHD.

In some aspects, the disclosure includes a regimen for treating GVHD in a subject, the regimen comprising: measuring a biomarker or a combination of biomarkers in a biological sample from a subject with GVHD or at risk of GVHD, wherein the biomarker or the combination of biomarkers is selected from the group consisting of REG3α, ST2, and REG3α and ST2, wherein an increased level of the biomarker or combination of biomarkers compared with control indicates that the subject is suffering from GVHD or is at risk of GVHD; and for a subject with GVHD or a risk, probability, or susceptibility of developing GVHD based upon level of the biomarker or the combination of biomarkers and presence or absence of the clinical parameter or the combination of clinical parameters, prescribing or administering a treatment regimen that includes a steroid, an immunosuppressant, or a combination of steroid and immunosuppressant.

In some aspects, the disclosure includes a regimen for treating GVHD in a subject, the treatment regimen comprising: measuring a biomarker or a combination of biomarkers in a biological sample from a subject at risk of GVHD, wherein the biomarker or the combination of biomarkers is selected from the group consisting of REG3α, ST2, and REG3α and ST2; assessing a clinical parameter or a combination of clinical parameters in the subject; and for a subject with a risk, probability, or susceptibility of developing GVHD based upon level of the biomarker or the combination of biomarkers and presence or absence of the clinical parameter or the combination of clinical parameters, prescribing or administering a treatment regimen that includes a steroid, an immunosuppressant, or a combination of steroid and immunosuppressant.

In some aspects, the clinical parameter or the combination of clinical parameters comprises any of the clinical parameters selected from the group consisting of: age of the subject; whether the subject received a bone marrow transplantation or a peripheral blood stem cell transplantation, whether all human leukocyte antigens were matched or mismatched in the transplant, whether subject received previous treatment with tacrolimus and methotrexate, whether subject received high toxicity conditioning without total body irradiation; and whether subject received high toxicity conditioning with or without total body irradiation.

In some aspects, the biomarker or the combination of biomarkers further comprises a biomarker or combination of biomarkers selected from the group consisting of elafin, TNFR1, IL2Rα, IL-8, and HGF. In some aspects, the combination of biomarkers comprises REG3α, elafin, TNFR1, and IL2Rα. In some aspects, the biomarker is REG3α. In some aspects, the biomarker is ST2.

In some aspects, the disclosure includes the use of measurement of an elevated level of a biomarker or a combination of biomarkers in a biological sample from a subject at risk of GVHD compared to control level, wherein the biomarker or the combination of biomarkers is selected from the group consisting of REG3α, ST2, and REG3α and ST2, for the selection of a treatment regimen for the subject. In some aspects, the use also comprises measurement of a clinical parameter or a combination of clinical parameters in the subject. In some aspects, the clinical parameter or the combination of clinical parameters comprises any of the clinical parameters selected from the group consisting of: age of the subject; whether the subject received a bone marrow transplantation or a peripheral blood stem cell transplantation, whether all human leukocyte antigens were matched or mismatched in the transplant, whether subject received previous treatment with tacrolimus and methotrexate, whether subject received high toxicity conditioning without total body irradiation; and whether subject received high toxicity conditioning with or without total body irradiation. In some aspects, the biomarker or the combination of biomarkers further comprises a biomarker or combination of biomarkers selected from the group consisting of elafin, TNFR1, IL2Rα, IL-8, and HGF. In some aspects, the combination of biomarkers comprises REG3α, elafin, TNFR1, and IL2Rα. In some aspects, the biomarker is REG3α. In some aspects, the biomarker is ST2.

In some aspects, the disclosure includes a method of decreasing toxicity of a regimen for treating GVHD in a subject diagnosed with GVHD, wherein the subject is being treated with a more aggressive therapy for GVHD comprising: measuring a level of a biomarker or a combination of biomarkers in a biological sample from the subject diagnosed with GVHD, wherein the biomarker or the combination of biomarkers is selected from the group consisting of REG3α, ST2, and REG3α and ST2; and wherein a decreased level of the biomarker or combination of biomarkers compared with control level indicates that the subject is at reduced risk of GVHD; and prescribing or administering to the subject a less aggressive therapy or regimen for treating GVHD. In some aspects, the biomarker or the combination of biomarkers further comprises a biomarker or combination of biomarkers selected from the group consisting of elafin, TNFR1, IL2Rα, IL-8, and HGF. In some aspects, the combination of biomarkers comprises REG3α, elafin, TNFR1, and IL2Rα. In some aspects, the biomarker is REG3α. In some aspects, the biomarker is ST2.

In various aspects of the disclosure, ST2, elafin, TNFR1, IL2Rα, IL-8, and HGF are expressed in pg/mL and REG3α is expressed in ng/mL.

In various aspects of the disclosure, the GVHD is acute GVHD. In particular aspects, the GVHD is acute GI GVHD.

In various aspects of the disclosure, the biological sample comprises whole blood, plasma, serum, stool, urine, emesis, or bronchoalveolar lavage fluid. In some aspects, the biological sample comprises plasma or serum.

In various aspects of the disclosure, the subject is a mammal. In some aspects, such mammal is a human. In particular aspects, the subject is suffering from GVHD. In other aspects, the subject is at risk of developing GVHD. In some aspects, the subject exhibits severe intestinal inflammation, sloughing of the mucosal membrane, severe or high-volume diarrhea, gastrointestinal bleeding, abdominal pain, nausea, anorexia or vomiting.

In various aspects of the methods of the disclosure, measuring of the biomarker is performed with an immunoassay, Northern blot analysis, or reverse transcription quantitative polymerase chain reaction. In some aspects, the immunoassay is an ELISA.

In some aspects, the disclosure includes a kit comprising reagents for measuring the biomarker or combination of biomarkers described herein. In particular aspects, such kits include components or reagents for measuring a biomarker or combination of biomarkers present in a biological sample isolated from the subject.

In some aspects, the disclosure includes a kit for assessing susceptibility of developing GVHD in a subject, the kit comprising reagents for selectively detecting a level of a biomarker or a combination of biomarkers in a biological sample from a subject, wherein the biomarker or the combination of biomarkers is selected from the group consisting of REG3α, ST2, or a combination of REG3α and ST2. In further aspects, the biomarker or the combination of biomarkers further comprises a biomarker or combination of biomarkers selected from the group consisting of elafin, TNFR1, IL2Rα, IL-8, and HGF. In more particular aspects, the biomarker or the combination of biomarkers is selected from the group consisting of REG3α, elafin, TNFR1, anti IL2Rα. In some aspects, the biomarker is REG3α. In some aspects, the biomarker is ST2. In some aspects, the reagents comprise an antibody that binds to the biomarker or antibodies that bind to the combination of biomarkers in the biological sample from the subject, a buffer, and a detectable label for identifying antibody binding to the biomarker or the combination of biomarkers. In some aspects, the kit further comprises a steroid and/or immunosuppressant used in the treatment of GVHD.

In various aspects, the disclosure includes methods, kits, systems, regimens, and uses of any one biomarker or combination of biomarkers listed in the Table of Biomarkers and Combinations of Biomarkers, disclosed herein, as illustrated in columns 1-42, for the prediction, diagnosis, and treatment of GVHD based upon the expression of the biomarker or a combination of biomarkers.

The foregoing summary is not intended to define every aspect of the disclosure, and additional aspects are described in other sections, such as the following detailed description. The entire document is intended to be related as a unified disclosure, and it should be understood that all combinations of features described herein are contemplated, even if the combination of features are not found together in the same sentence, or paragraph, or section of this document. Other features and advantages of the invention will become apparent from the following detailed description. It should be understood, however, that the detailed description and the specific examples, while indicating specific embodiments of the disclosure, are given by way of illustration only, because various changes and modifications within the spirit and scope of the disclosure will become apparent to those skilled in the art from this detailed description.

### BRIEF DESCRIPTION OF THE DRAWINGS

The following Detailed Description, given by way of example, but not intended to limit the invention to specific embodiments described, may be understood in conjunction with the accompanying Figures, incorporated herein by reference, in which:

FIG. 1 depicts REG3α concentrations in plasma samples from HCT patients, i.e. human subjects of two independent validation sets: (A) University of Michigan subjects (n=871); (B) Regensburg, Germany, and Kyushu, Japan (n=143); and (C) Plasma REG3α concentrations in subjects classified by GI symptoms and histologic diagnosis and categorized by conditioning regimen intensity. High intensity regimens included: cyclophosphamide±cytarabine, thiotepa, fludarabine, and/or total body irradiation (TBI); cyclophosphamide/etoposide phosphate (VP-16)/bis-chloroethylnitrosourea (BCNU); busulfan+cytarabine, clofarabine, melphalan, cyclophosphamide/anasacrin or cytarabine/cyclophosphamide; BCNU/VP-16/cytarabine/melphalan; TBI±VP-16; melphalan. Moderate intensity regimens included: fludarabine+busulfan or treosulfan±TBI, melphalan; zevalin or anasacrin/cytarabine; fludarabine±TBI, melphalan, or cyclophosphamide; fludarabine/BCNU/melphalan; TBI. (D) Subjects classified by symptoms and etiology (n=675).

FIG. 2 depicts ROC curves for human subjects with post-HCT diarrhea. ROC curves comparing REG3α concentrations for subjects with diarrhea caused by GVHD (n=162) and not caused by GVHD (N=42). REGα alone: AUC=0•80; IL2Rα: AUC=0•69; Elafin: AUC=0•68; IL-8: AUC=0•61; HGF: AUC=0•61; TNFR1: AUC=0•60; Composite of all 6 biomarkers: AUC=0•81.

FIG. 3 depicts REG3α expression according to severity of GVHD at diagnosis. Human subjects were classified by volume of diarrhea (A) and histologic grade (B).

FIG. 4 shows a correlation of Paneth cells per high-powered field with histologic severity. Number of Paneth cells observed per high-powered field (y-axis) in randomly selected biopsies from subjects with onset histologic grade 4 (N=12), onset histologic grade 3 (N=10), and onset histologic grade 0 (non-GVHD enteritis; N=10).

FIG. 5 depicts the prognostic value of REG3α concentrations at onset of GVHD. (A) Human subjects were classified by response to GVHD therapy after 4 weeks (N=160). (B to D) Subjects were classified by REG3α concentration: low (≦151 ng/ml, n=81; thin line) and high (>151 ng/ml, n=81; thick line). (B) NRM (34% versus 59%, p<0•001) (C) Relapse mortality (17% versus 14%, p=0•59). (D) 1-year survival (48% versus 27%, p=0•001). All p-values are adjusted for donor source, HLA-match, conditioning intensity, recipient age and baseline disease severity according to the Center for International Blood and Marrow Transplant Research (CIBMTR) guidelines. (E) 1 year NRM for subjects classified by number of risk factors at GVHD onset, using clinical stage (high risk=stage 2-4) and histologic grade (high risk=grade 4). 0 (NRM=26%); 1 (NRM=60%); 2 (NRM=71%). 0 vs. 1, p<0.001; 1 vs. 2, p=0•006. (F) 1 year NRM for subjects classified by number of risk factors at the time of GVHD diagnosis as in E and including REG3α concentration (high risk>151 ng/ml). 0 (NRM=25%); 1 (NRM=34%); 2 (NRM=66%); 3 (NRM=86%). 0 vs. 1, p=0•2; 1 vs. 2, p<0•001; 2 vs. 3, p<0•001.

FIG. 6 depicts the identification of REG3α through discovery phase proteomics. MS/MS of the identified peptide; REG3α. Bₙ or yₙ denotes the fragment ion generated by cleavage of the peptide bond after the nth amino acid containing either the peptide N terminus (b series) or the C terminus (y series), respectively. The identified b and y ions and all fragment ion (m/z) values are indicated in the table. C* denotes cysteine residues modified by acrylamide containing three ¹³C atoms. The identified peptide sequence location is underlined within the protein sequence.

FIG. 7 shows REG3α concentrations in the discovery set. Plasma concentrations of REG3α were measured by ELISA in the 20 individual samples of the discovery set, and are presented as scatter plots with lines for means.

FIG. 8 shows ROC curves for two independent validation sets. ROC curves comparing plasma REG3α concentrations in subjects with diarrhea caused by GVHD (N=162) and not caused by GVHD (N=42). University of Michigan validation set (thick line), AUC=0.76; Regensburg/Kyushu validation set (thin line), AUC=0•79.

FIG. 9 depicts albumin concentrations by severity of lower GI GVHD diarrhea. (A) Serum albumin correlation by clinical lower GI GVHD stage. Stage 1 (N=67) versus stage 2-4 (N=73), p=0•005. (B) Serum albumin concentrations and histologic grade. Histologic grade 1-3 (intact mucosa, N=107) versus grade 4 (denuded mucosa; N=33), p=0•04.

FIG. 10 depicts the correlation of REG3α concentrations at onset of lower GI GVHD correlate with eventual maximum GVHD severity. Plasma REG3α concentrations in subjects with lower GI GVHD at onset (y-axis) are compared between subjects with maximum GVHD severity of grade 2 (N=49) and subjects who eventually developed maximum grade 3-4 GVHD (N=113), p<0•001.

FIG. 11 depicts a diagram illustrating a system comprising computer implemented methods utilizing risk scores and probability as described herein.

FIG. 12 depicts an exemplary system for determining risk of GVHD as described further herein.

FIG. 13 depicts an exemplary system for selecting a treatment protocol for a subject diagnosed with GVHD or at risk of GVHD.

### DETAILED DESCRIPTION

The disclosure relates to the identification of a biomarker associated with a subject having GVHD or a subject at risk of having GVHD and therefore provides methods of determining a subject's need for GVHD prophylaxis or treatment. More particularly, the disclosure features methods for identifying subjects who either have developed, or are at risk of developing, GI GVHD, by detection of the biomarker or combination of biomarkers disclosed herein. Such biomarker(s) is also useful for monitoring subjects undergoing treatments and therapies for GI GVHD, and for selecting or modifying therapies and treatments that would be efficacious in subjects having GI GVHD, wherein selection and use of such treatments and therapies slow the progression of GI GVHD, and/or prevents its onset.

More specifically, the disclosure provides fast and robust methods of detecting or predicting GI GVHD by measuring an elevated level of regenerating islet-derived 3-alpha (REG3α) in a biological sample from a subject suffering from or at risk of suffering from GI GVHD.

Before any embodiments of the subject matter of the disclosure are explained in detail, it is to be understood that the disclosure is not limited in its application to the details of construction and the arrangement of components set forth in the following description or illustrated in the figures and examples. Accordingly, the disclosure embraces other embodiments and is practiced or carried out in various ways.

The section headings as used herein are for organizational purposes only and are not to be construed as limiting the subject matter described.

### DEFINITIONS

To aid in understanding the detailed description of the compositions and methods according to the disclosure, a few express definitions are provided to facilitate an unambiguous disclosure of the various aspects of the disclosure.

Unless otherwise defined, all technical and scientific terms used herein have the same meaning as commonly understood by one of ordinary skill in the art to which this disclosure belongs.

The following abbreviations are used throughout.

AA Amino acid
AUC Area under curve

### BCNU Bis-chloroethylnitrosourea

BMT Bone marrow transplant
CI Confidence interval
CR Complete response
CV Coefficient of variation
ELISA Enzyme-linked immunosorbent assay
GI GVHD Gastrointestinal graft-versus-host disease
GVHD Graft-versus-host disease
HLA Human leukocyte antigen
HCT Hematopoietic cell transplantation
HGF Human growth factor
HPF High-power field

### IL-8 Interleukin 8

IL2α Interleukin-2 receptor alpha
ISC Intestinal stem cells
IPAS Intact protein analysis system
IPS Idiopathic pneumonia syndrome
LLOD Lower limit of detection

### μM Micromolar

### M Molar

### mL Milliliter

### mM Millimolar

MS Mass spectrometry
MS/MA Tandem mass spectrometry

### NG Nanogram

NRM Non-relapse mortality
N/V Nausea and vomiting
OS Overall survival

### PG Picogram

PR Partial response
REG3α Regenerating islet-derived 3-alpha
RNA Ribonucleic acid
ROC Receiver operating characteristic
SEM Standard error of the mean
SOS Sinusoidal obstruction syndrome
ST2 IL33 receptor
TBI Total body irradiation
TNFR1 Tumor necrosis factor receptor 1
ULOD Upper limit of detection
VP-16 Etoposide phosphate

It is noted here that, as used in this specification and the appended claims, the singular forms “a,” “an,” and “the” include plural reference unless the context clearly dictates otherwise. The terms “including,” “comprising,” “containing,” or “having” and variations thereof are meant to encompass the items listed thereafter and equivalents thereof as well as additional subject matter unless otherwise noted.

A “control,” as used herein, refers to an active, positive, negative or vehicle control. As will be understood by those of skill in the art, controls are used to establish the relevance of experimental results, and provide a comparison for the condition, e.g., level or amount of biomarker, being tested.

“Measuring” or “measurement” means assessing the presence, quantity or level of a substance, e.g. a biomarker, within a clinical or subject-derived sample, including the derivation of qualitative or quantitative concentration levels of such substance, or otherwise evaluating the values or categorization of a subject's clinical parameters. Recitation of ranges of values herein are merely intended to serve as a shorthand method for referring individually to each separate value falling within the range and each endpoint, unless otherwise indicated herein, and each separate value and endpoint is incorporated into the specification as if it were individually recited herein.

The terms “level” and “amount” are used herein interchangeably to mean the concentration of biomarker present in a biological sample.

A “biomarker” in the context of the disclosure encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. In some aspects, therefore, a biomarker includes a protein or a fragment thereof or a nucleic acid or a fragment thereof. In exemplary aspects, the biomarker is REG3α. In additional aspects, one or more biomarkers are measured together to provide an array for the diagnosis or prediction of a particular disease or condition, such as GI GVHD.

The term “REG3α,” as used herein, refers to a “regenerating islet-derived 3-alpha” protein or nucleic acid.

The term “IL2Rα” as used herein, refers to an “interleukin 2 receptor alpha” protein or nucleic acid.

The terms “TNFRSF1A or TNFR1,” as used herein, refer to a “tumor necrosis factor receptor superfamily member 1A” protein or nucleic acid.

The term “IL-8,” refers to an “interleukin 8” protein or nucleic acid.

The term “HGF,” as used herein, refers to a “hepatocyte growth factor” protein or nucleic acid.

The term “elafin” as used herein, refers to an “elafin” protein or nucleic acid.

The term “ST2” as used herein, refers to an “ST2” protein or nucleic acid.

The terms “protein,” “polypeptide,” and “peptide” are used interchangeably herein to refer to a polymer of amino acid residues linked via peptide bonds. The term “protein” typically refers to large polypeptides. The term “peptide” typically refers to short polypeptides.

The term “nucleic acid” or “nucleic acid sequence” or “nucleic acid molecule” refers to deoxyribonucleotides or ribonucleotides and polymers thereof in either single- or double-stranded form. The term nucleic acid is used interchangeably with gene, complementary DNA (cDNA), messenger RNA (mRNA), oligonucleotide, and polynucleotide.

As used herein, a “fragment” of a protein or a nucleic acid refers to any portion of the protein or nucleic acid smaller than the full-length protein, nucleic acid, or protein expression product. Fragments are deletion analogs of the full-length protein or nucleic acid wherein one or more amino acid residues (protein) or nucleotides (nucleic acid) have been removed from the amino terminus (protein) or 5′ end (nucleic acid) and/or the carboxy terminus (protein) or 3′ end (nucleic acid) of the full-length protein or nucleic acid.

As used herein, the term “subject” refers to a mammal who is at risk of developing GVHD or who suffers from GVHD. Such mammals include, but are not limited to, mammals of the order Rodentia, such as mice and rats, and mammals of the order Logomorpha, such as rabbits, mammals from the order Carnivora, including felines (cats) and canines (dogs), mammals from the order Artiodactyla, including bovines (cows) and swines (pigs) or of the order Perssodactyla, including equines (horses), mammals from the order Primates, Ceboids, or Simoids (monkeys) and of the order Anthropoids (humans and apes). In various aspects, mammals other than humans are advantageously used as subjects that represent animal models of GVHD. In exemplary aspects, the mammal is a human.

The term “treatment” as used herein includes all treatments, therapies, or therapeutic agents used in the art for treating GVHD. Thus, “treatment,” as used herein includes administration of one or more therapeutic agents for GVHD, including first and second line GVHD therapeutic agents.

As used herein, a “biological sample” taken from a subject is, in various aspects, any sample (e.g., solid, liquid, or gas) obtained from the subject, including, but not limited to, exhaled air, breath condensate, tissue, cells, cell extracts, whole blood, plasma, serum, inflammatory fluids, stool (e.g., feces), urine, semen, cerebrospinal fluid, lymph (e.g., endolymph, perilymph), gastric juice, mucus, peritoneal fluid, pleural fluid, bronchoalveolar lavage fluid, sebum, sweat, tears, vaginal secretion, emesis, breastmilk, amniotic fluid, bile, cerumen, and saliva. In exemplary embodiments, the biological sample is whole blood, plasma, serum, stool, urine, emesis, or bronchoalveolar lavage fluid. In various aspects, the biological sample or “sample” contains nucleic acid and/or protein and/or fluid containing organic and/or inorganic metabolites and substances. In exemplary aspects of the invention, the sample comprises protein suitable for protein level or protein expression level analysis.

The term “susceptibility” or “risk,” as used herein, refers to the proneness of a subject towards the development of GVHD, or towards being less able to resist development of GVHD than the average subject. The term encompasses both increased susceptibility or risk and decreased susceptibility or risk. Thus, in certain aspects, an increased level of a biomarker or a combination of biomarkers compared to control indicates an increased susceptibility or increased risk. Likewise, a decreased level of a biomarker or a combination of biomarkers compared to control indicates a decreased susceptibility or decreased risk. In more particular aspects, a level of a biomarker or combination of biomarkers is characteristic of increased susceptibility (i.e., increased risk), and the susceptibility is further characterized by a probability (p) of developing GVHD. In certain aspects, the susceptibility or risk is determined by additionally assessing various clinical parameters of the subject.

In some aspects, the probability (p) ranges from 0 to 1, wherein 0 indicates no risk and 1 equals a 100% risk, i.e., of the development of GVHD. Thus, in specific aspects, a probability of 0.33 indicates a 33% risk of GVHD, and a probability of 0.38 indicates a 38% risk. As the probability increases toward 1.0, there is an increased risk of developing GVHD. As the probability decreases toward 0, there is an decreased risk of developing GVHD. In exemplary aspects, a probability of greater than or equal to about 0.33 for a subject who received an unrelated donor transplant, or a probability of greater than or equal to about 0.38 for a subject who received a related donor transplant, is indicative of an increased susceptibility or risk for GVHD in the subject. In other aspects, a probability of less than 0.33 for a subject who received an unrelated donor transplant, or less than 0.38 for a subject who received a related donor transplant, is indicative of a decreased susceptibility (i.e., decreased risk) of GVHD in the subject.

In particular aspects of the disclosure, a subject who is at risk of development of GVHD or has an increased susceptibility or risk of GVHD based upon a probability is treated for GVHD. In more particular aspects, a subject receives more aggressive therapy when they demonstrate an increased risk of GVHD and/or when their probability increases toward 1.0 (i.e., greater than about 40%, about 50%, about 60%, about 70%, about 80%, about 90%, or about 100%). In further aspects, a subject receives less aggressive therapy or no therapy when they demonstrate a decreased risk of GVHD and/or when their probability decreases toward 0 (i.e., lesser than about 30%, about 20%, or about 10%).

The term “and/or” is understood to indicate that either one or both of the items connected by it are involved. In other words, the term herein means “one or the other or both.”

The term “clinical parameter” refers to medical information or a personal characteristic of a subject including race, ethnicity, sex, age, behaviors and lifestyle (tobacco consumption (smoking), alcohol consumption (drinking), exercise, body mass indices), glucose tolerance/diabetes, particular genetic loci, disease state, and any other factors that medical personnel may measure in the context of standard medical care or specific diagnoses, including transplant information, and treatment information. In some aspects, a clinical parameter refers to medical information including whether the subject received a bone marrow transplantation or a peripheral blood stem cell transplantation, whether all human leukocyte antigens were matched or mismatched in the transplant, whether subject received previous treatment with tacrolimus and methotrexate, whether subject received high toxicity conditioning without total body irradiation; and whether subject received high toxicity conditioning with or without total body irradiation.

The term “look-up table”, as described herein, is a table that correlates one form of data to another form, or one or more forms of data to a predicted outcome to which the data is relevant, such as phenotype or trait. For example, a look-up table can comprise a correlation between biomarker expression level data for at least one biomarker and a particular trait or phenotype, such as a particular disease diagnosis, that an individual who comprises the particular biomarker expression level data is likely to display, or is more likely to display than individuals who do not comprise the particular biomarker expression level data. Look-up tables can be multidimensional, i.e. they can contain information about expression level data (either protein or nucleic acid data) for one or more biomarkers, and they may also comprise other factors, such as particulars about diseases, diagnoses, age, racial information, transplant information, biochemical information, and treatment information, including drugs, and the like.

The term “database” or “susceptibility database” refers to a collection of data organized for one or more purposes. In the context of the invention, databases may be organized in a digital format for access, analysis, or processing by a computer. The data are typically organized to model features relevant to the invention. For instance, one component of data in a database may be information about variations in a population, such as biomarker expression level variation with respect to various biomarkers, including, for example, regenerating islet-derived 3-alpha (REG3α), elafin, tumor necrosis factor receptor 1 (TNFR1), interleukin-2 receptor alpha chain (IL2Rα), interleukin 8 (IL-8), hepatocyte growth factor (HGF), ST2, and the like, but also variation with respect to other medically informative or clinical parameters, including race, ethnicity, sex, age, behaviors and lifestyle (tobacco consumption and/or smoking, alcohol consumption (drinking), exercise, and body mass indices), glucose tolerance/diabetes, genetic loci, and any other factors that medical personnel may measure in the context of standard medical care or specific diagnoses, including transplant information, and treatment information. Other components of the database may include one or more sets of data relating to susceptibility to a disease in a population, and/or suitability or success of a disease treatment, and/or suitability or success of a protocol for screening for or presenting a disease. Preferably the data is organized to permit analysis of how the biological variation in the population correlates with the susceptibility to disease and/or the suitability or success of the treatment, protocol, and the like. A look-up datable (or the information in a look-up table) may be stored in a database to facilitate aspects of the invention.

A “computer-readable medium” is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, and the like.

A “system” includes one or more components comprising at least one computing device and other components suitable for determining susceptibility or risk of developing GVHD in a subject.

The terms “bone marrow transplantation” and “peripheral blood stem cell transplantation” refer to different procedures that restore stem cells that were destroyed by high doses of chemotherapy and/or radiation therapy. After being treated with high-dose anticancer drugs and/or radiation, the patient receives the harvested stem cells, which travel to the bone marrow and begin to produce new blood cells.

The term “GVHD,” as used herein, refers to a “graft-versus-host disease.” GVHD is a complication that can occur after a stem cell or bone marrow transplant in which the newly transplanted material attacks the transplant recipient's, i.e., the subject's, body.

“Acute GVHD” refers to GVHD which usually occurs within about the first 100 days after transplant. “Chronic GVHD” usually occurs about more than 100 days after transplant and can last a lifetime. However, an “overlap” syndrome has recently been recognized in which diagnostic or distinctive features of acute GVHD and chronic GVHD appear together. “GI GVHD” refers to acute GVHD of the GI tract.

### Graft-Versus-Host Disease (GVHD)

In various aspects, the disclosure includes methods of detecting and/or predicting GVHD in a subject who has undergone transplantation and, therefore, the subject is at risk of developing GVHD. After bone marrow transplantation, T cells present in the graft, either as contaminants or intentionally introduced into the host, attack the tissues of the transplant recipient after perceiving host tissues as antigenically foreign. The T cells produce an excess of cytokines, including TNF-α and interferon-gamma (IFNγ). A wide range of host antigens can initiate GVHD, among them the human leukocyte antigens (HLAs). However, GVHD can occur even when HLA-identical siblings are the donors. HLA-identical siblings or HLA-identical unrelated donors often have genetically different proteins (called minor histocompatibility antigens) that can be presented by major histocompatibility complex (MHC) molecules to the donor's T-cells, which see these antigens as foreign and so mount an immune response.

In various aspects, such GVHD is acute or chronic GVHD. In the classical sense, acute GVHD is characterized by selective damage to organs and tissues including, but not limited to, the liver, skin (rash), mucosa, and gastrointestinal (GI) tract. Chronic GVHD also attacks the above organs, but over its long-term course also is known to cause damage to the connective tissue and exocrine glands. GI GVHD can result in severe intestinal inflammation, sloughing of the mucosal membrane, severe or high-volume diarrhea, gastrointestinal bleeding, abdominal pain, nausea, anorexia and vomiting. Until the present disclosure, GI GVHD has typically been diagnosed via intestinal biopsy.

Acute GVHD is staged as follows: overall grade (skin-liver-gut) with each organ staged individually from a low of 1 to a high of 4. A human subject with grade IV GVHD usually has a poor prognosis. If the GVHD is severe and requires intense immunosuppression involving steroids and additional agents to get it under control, a subject may develop severe infections as a result of the immunosuppression and may die of infection.

### Methods of Determining Susceptibility to GVHD

In one aspect, the disclosure provides a method of analyzing data representative of a biomarker of GVHD or a combination of biomarkers of GVHD in a subject, wherein the biomarker or combination of biomarkers is associated with a susceptibility to GVHD, and determining a susceptibility to GVHD for the subject from the data. In certain embodiments, the method is predictive of susceptibility of acute GVHD. In particular embodiments, the acute GVHD is acute gastrointestinal GVHD.

The data can be any type of data that is representative of the presence of the biomarker. In certain embodiments, the data is protein biomarker data or nucleic acid biomarker data. In exemplary embodiments, the protein biomarker data is biomarker protein expression level data or biomarker protein level data. In certain embodiments, the biomarker protein level data is obtained from a biological sample comprising or containing protein from a subject. In some embodiments, the biomarker protein level data is obtained using any method known for analyzing protein data in a biological sample. In other embodiments, the biomarker protein level data is obtained from a preexisting record. For example, the preexisting record may comprise a protein dataset for a biomarker or a combination of biomarkers. In certain embodiments, the determining comprises comparing the biomarker data to a database containing correlation data between the biomarker or combination of biomarkers and susceptibility to GVHD. In certain embodiments, the biomarker data is provided as protein level, identifying the level of the biomarker or the combination of biomarkers present in the biological sample.

The data to be analyzed by the methods of the disclosure is suitably obtained by analysis of a biological sample from a subject to obtain information about the levels of biomarkers present in the blood of the subject. In certain embodiments, the information is measurement of protein expression level information or nucleic acid expression level information.

In a further embodiment of the disclosure, a biological sample is obtained from the subject prior to the analyzing steps. The analyzing may also suitably be performed by analyzing data from a preexisting record about the subject. The preexisting record may, for example, include data regarding biomarker expression level in the subject.

In certain embodiments, information about risk for developing GVHD in the subject can be determined using methods known in the art. Some of these methods are described herein. For example, information about the probability of developing GVHD is determined from information about the protein expression level of a biomarker or a combination of biomarkers.

It is contemplated that in certain embodiments of the disclosure, it may be convenient to prepare a report of results of risk assessment. Thus, certain embodiments of the methods of the disclosure comprise a further step of preparing a report containing results from the determination of risk, wherein said report is written in a computer readable medium, printed on paper, or displayed on a visual display. In certain embodiments, it may be convenient to report results of susceptibility to at least one entity selected from the group consisting of the subject, a guardian of the subject, a physician, a medical organization, and a medical insurer.

Risk Assessment and Formulas for Predicting GVHD after Transplant

Within any given population, there is an absolute risk of developing a disease or trait, defined as the chance of a person developing the specific disease or trait over a specified time-period. Formulas were developed for predicting probability or risk of GVHD in a patient by calculating a score (or risk score) and then determining a probability from that score from data collected from a pool of over 800 patients. The formulas comprise data from biomarker analysis along with various clinical parameters. Data from the biomarker analysis and collection of clinical parameters is factored into a formula for calculation of a score for each patient. Such clinical parameters and patient characteristics include patient age, type of transplantation (i.e., bone marrow versus peripheral blood stem cell), matching of human leukocyte antigen (HLA) loci, whether patient received treatment with both tacrolimus and methotrexate, whether patient received a high toxicity conditioning regimen, and whether patient did or did not receive total body irradiation.

High toxicity conditioning in a patient is an intense, myeloablative conditioning regimen prior to HCT aimed at reducing tumor burden. Such myeloablative conditioning is described by the Center for International Blood and Marrow Transplant Research (CIBMTR) and is defined in the literature (Bacigalupo et al., Biol. Blood Marrow Transplant. 15:1628-1633, 2009). Total body irradiation (TBI) is considered to have been administered to a patient if the patient received a dose of TBI greater than about 500 centigrade. If the dosage of radiation was less than about 500 centigrade, the patient was considered to be without TBI.

A patient receives a “score” equal to A+B, wherein “A” is computed from biomarker data and “B” is computed from clinical parameter data of the patient Each patient's score is then converted to a predicted probability (p) of GVHD using the following formula:

$$p = \frac{{e}^{score}}{1 + {e}^{score}}$$

so that “p” will lie somewhere between 0 and 1. Each patient then gets a score based on the sum of the different factors as shown in the formulas below. Different formulas are used depending on whether the transplant was from a related donor or an unrelated donor.

To compute “A” in the formula, the following clinical observations and/or patient characteristics/variables are recorded and are inputed in the formula:

Age=1 if patient's age >55 yo; age=0 if patient's age <=55 yo;
BM (bone marrow)=1 if bone marrow transplantation; BM=0 if peripheral blood transplantation;
Mismatch=1 if patient does not match all, i.e., eight of eight HLA loci, 2 genes for each of the four loci, HLA-A, B, C, and DR, with the transplant; mismatch=0 if patient matches all eight loci;
TM=1 if patient received both tacrolimus (Tacro) and methotrexate (MTX); TM=0 if patient did not receive both Tacro and MTX;
Tox1=1 if patient received high toxicity conditioning without total body irradiation (TBI); Tox1=0 if patient did not receive high toxicity conditioning without TBI; and
Tox2=1 if patient received high toxicity conditioning with TBI; Tox2=0 if patient does not receive high toxicity conditioning with TBI.

To compute “B” in the formula, protein concentrations (in pg/ml for IL2Rα, TNFR1 and elafin; and in ng/ml for Reg3α) of biomarkers is measured in a biological sample from each patient one week after transplant.

### A) Related Donor Transplants

A recipient of a related donor transplant will receive a “score” equal to A+B, wherein

A=−3.57+0.54×Age−16.83×BM+1.35×Mismatch−0.08×TM+0.35×Tox1+0.47×Tox2,

wherein the values of “0” or “1” are multiplied by a conversion factor to determine “A;” and wherein

B=0.37×log IL2Rα−0.06×log TNFR1−0.12×log Elafin−0.03×log Reg3α,

wherein the log base 2 of each biomarker protein level (ng/ml) is multiplied by a conversion factor to determine “B.”

Each patient's score is then converted to a predicted probability, p, of GVHD using the following formula:

$$p = \frac{{e}^{score}}{1 + {e}^{score}}$$

so that “p” will lie somewhere between 0 and 1.

For related donors, a patient is determined to have a positive test result, i.e., a positive test result for predicting GVHD, if their p value is above 0.38.

### B) Unrelated Donor Transplants

A recipient of an unrelated donor transplant will receive a “score” equal to A+B, wherein

A=−1.87+0.16×Age+0.23×Match+−0.28×TM+0.18×Tox1+1.25×Tox2

wherein the values of “0” or “1” are multiplied by a conversion factor to determine “A;” and wherein

B=0.86×log IL2Rα−0.49×log TNFR1−0.23×log Elafin+0.06×log Reg3α

wherein the log base 2 of each biomarker protein level (ng/ml) is multiplied by a conversion factor to determine “B.”

The variables, explained in more detailed herein above, that were used to compute “A” are as follows:

### Age=1 if age >55 yo & 0 if age 55 yo

Match=1 if matched & 0 if mismatched
TM=1 if Tacro/MTX given & 0 if Tacro/MTX not given
Tox1=1 if given high toxicity conditioning without TBI & 0 otherwise
Tox2=1 if given high toxicity conditioning with TBI & 0 otherwise

Each patient's score was then converted to a predicted probability, p, of GVHD using the following formula:

$$p = \frac{{e}^{score}}{1 + {e}^{score}}$$

so that “p” will lie somewhere between 0 and 1.

A patient is then determined to have a positive test result, i.e., probability or risk of GVHD, if their value of p is above about 0.33.

### Database

Determining susceptibility can alternatively or additionally comprise comparing protein expression level data (or nucleic acid expression level data) to a database containing correlation data between biomarker expression level data and susceptibility to GVHD. The database can be part of a computer-readable medium described herein.

In a specific aspect of the disclosure, the database comprises at least one measure of susceptibility to GVHD for the biomarker or combination of biomarkers. For example, the database may comprise risk values associated with particular expression levels of such biomarker or risk values associated with particular combinations of biomarkers.

In another specific aspect of the disclosure, the database comprises a look-up table containing at least one measure of susceptibility to GVHD for the biomarker or combination of biomarkers.

### Further Steps

The methods disclosed herein can comprise additional steps which may occur before, after, or simultaneously with one of the aforementioned steps of the method of the disclosure. In a specific embodiment of the disclosure, the method of determining a susceptibility to GVHD further comprises reporting the susceptibility to at least one entity selected from the group consisting of the subject, a guardian of the subject, a physician, a medical organization, and a medical insurer. The reporting may be accomplished by any of several means. For example, the reporting can comprise sending a written report on physical media or electronically or providing an oral report to at least one entity of the group, wherein the written or oral report comprises the susceptibility. Alternatively, the reporting can comprise providing the at least one entity of the group with a login and password, which provides access to a report comprising the susceptibility posted on a password-protected computer system.

### Study Population

The methods, kits, systems, regimens, and uses described herein can be utilized from samples containing protein or nucleic acid material (DNA or RNA) from any source and from any subject. The disclosure also provides for assessing biomarker expression level in subjects who are members of a target population. Such a target population is in one embodiment a population or group of subjects at risk of developing GVHD.

Methods of Selecting Subjects for Treatment

In one aspect of the disclosure, a method for treating GVHD in a subject suffering from GVHD is provided, wherein the method comprises the steps of identifying the subject at risk of suffering from GVHD, measuring a level of a biomarker or a combination of biomarkers in a biological sample isolated from the subject, wherein an increased level of the biomarker present in the biological sample compared to a control level indicates GVHD in the subject, and administering an effective amount of a treatment for GVHD to the subject.

Methods of the disclosure relating to identifying a subject for treatment may further include a step of administering a therapeutic regimen to the subject. Methods of the disclosure relating to identifying subjects for treatment may further include a step of prescribing the therapeutic for the subject for self-administration, or for administration by a medical professional other than the professional that selects the patient.

### Prognostic Methods and Methods of Treatment

In addition to the utilities described above, the biomarker or combination of biomarkers of the disclosure are useful in determining efficacy of a treatment for GVHD. The disclosure includes methods for determining efficacy of a treatment for GVHD in a subject suffering from GVHD, wherein the method comprises administering to the subject the treatment for GVHD, and measuring a level of biomarker or a combination of biomarkers in a biological sample obtained from the subject, wherein a decrease in the level of the biomarker relative to the level of the biomarker prior to administration of the treatment, indicates that the treatment is effective for treating GVHD in the subject.

It may be useful to select subjects for treatment based on increased expression of a biomarker or a combination of biomarkers. It also may be useful to select subjects for treatment based on biomarker expression along with the presence or absence of a variety of clinical parameters as discussed herein. Accordingly, the disclosure provides in one aspect a method of treatment of GVHD in a subject suffering from GVHD, wherein the method comprises the steps of measuring a level of a biomarker or a combination of biomarkers in a biological sample isolated from the subject, and wherein an increased level of the biomarker or combination of biomarkers present in the biological sample compared to a control level indicates GVHD in the subject, and administering an effective amount of a treatment for GVHD to the subject.

### Treatment of GVHD

In various aspects, the disclosure includes methods of treating GVHD. To date, several successful strategies have been used to reduce the risk of developing acute GVHD. Such strategies include prophylaxis with immunosuppressive drugs, selective depletion of alloreactive T lymphocytes from the donor graft, the use of umbilical cord blood as a source of donor cells, and choosing more closely HLA-matched donors.

If acute GVHD does develop after transplantation, one or more immunosuppressive drugs are administered. The disclosure includes such methods for treating GVHD after detecting or diagnosis of GVHD or detecting a risk of GVHD by an increased level of a biomarker or a combination of biomarkers.

Typically, the first line treatment for GVHD is the administration of steroids and the second line treatment for GVHD is the administration of immunosuppressive drugs. In some aspects, however, steroids are administered with immunosuppressive drugs at the onset of GVHD. Such steroids include, but are not limited to, corticosteroids (e.g., prednisone, prednisolone, methylprednisolone, and the like). Such immunosuppressive drugs include, but are not limited to, cyclosporine, tacrolimus (also known as FK-506 or Fujimycin), methotrexate, mycophenoate mofetil, antithymocyte globulin (ATG), monoclonal antibodies (e.g., anti-CD3, -CD5, and -IL-2 antibodies, anti-CD20 (rituximab), and alemtuzumab (Campath)), anti-TNF drugs (e.g., etanercept (Enbrel®), infliximab, adlimumab), lymphocyte immune globulin (Atgam®), sirolimus, ustekinumab, extracorporeal photophoresis (ECP), anti-CD3 drugs (e.g., Visilizumab and OKT3), anti-CD5 drug and anti-IL-2(CD25) drugs (inolimomab, basiliximab, daclizumab, and denileukin diftitox), anti-CD147 drugs (e.g., Alefacept), anti-IL1R drugs, (e.g., Anakinra), mesenchymal stem cells, and regulatory T cells. The list of drugs provided herein above is not meant to be limiting as a person skilled in the art is aware of the many available treatment options for GVHD, acute GVHD, and GI GVHD. The disclosure includes methods of treatment for GVHD as discussed by Blazar et al. in Nature Reviews Immunology 12: 443-58, 2012.

In some aspects, high-level steroid doses are administered if a subject is considered to be high risk or demonstrates an increased risk of GVHD. In some aspects, these high steroid doses are combined with immunosuppressive drugs. In some aspects, high steroid doses alone or combined with immunosuppressive drugs is considered a more aggressive therapy or regimen. In some aspects, low-level steroid doses are administered or no steroid treatment is administered if a subject is considered to be low risk or demonstrates a decreased risk of GVHD. In some aspects, low-level steroid doses alone or combined with immunosuppressive drugs is considered a less aggressive therapy or regimen. In more particular aspects, “decreasing toxicity” of a therapy or regimen for the treatment of GVHD may include such practices as reducing drug dosage or changing GVHD therapy to a less toxic drug and/or a less toxic combination of drugs.

The GVHD treatment may be administered to the subject via any suitable route of administration. The effective amount or dose of GVHD treatment administered should be sufficient to provide a therapeutic or prophylactic response in the subject over a reasonable time frame. For example, the dose of immunosuppressive drug should be sufficient to decrease symptoms of GVHD along with decreasing the level of any of the biomarkers described herein as being associated with GVHD. The dose will be determined by the efficacy of the particular active agent and the condition of the subject (e.g., human), as well as the body weight of the subject (e.g., human) to be treated.

### Determining Efficacy of Therapeutic Agents

The disclosure also provides methods of determining the efficacy of a therapeutic agent in treating GVHD. In exemplary aspects, the method comprises the steps of administering to a subject suffering from GVHD a therapeutic agent used in the treatment of GVHD, and measuring a level of biomarker in a biological sample obtained from the subject, wherein a decrease in the level of biomarker relative to the level prior to administration of the therapeutic agent, is indicative of the therapeutic agent as effective for decreasing GVHD in a subject.

In additional aspects of the disclosure, methods are providing for predicting outcome for a subject at the onset of GVHD by measuring the level of a biomarker. In such aspects, the methods of the disclosure are useful because

The step of administering an effective amount of a therapeutic agent to the subject occurs through any suitable route of administration known in the art, some of which are described herein. In exemplary aspects, the step of administering an effective amount of a therapeutic agent to the subject comprises administering a therapeutic agent to the subject. For example, a typical first line therapy for GVHD is the administration of steroids including, but not limited to, corticosteroids (such as, prednisone, prednisolone, and methylprednisolone) at a dosage of about 1-2 mg/kg/day. If a response to first line therapy is not seen, immunosuppressive drugs are administered. In some aspects, subjects are treated with both steroids and immunosuppressive drugs at the onset of GVHD.

The therapeutic agent may be any suitable agent and in exemplary aspects is a therapeutic agent which is effective or is being evaluated for its efficacy as a treatment for GVHD. In a particular aspect, the therapeutic agent is REG3α. REG3α has been shown to reduce inflammation of human intestinal crypts in vitro, and its administration protects ISCs and prevents GI epithelial damage.

### Biomarkers

In various aspects, the disclosure includes methods of measuring a biomarker in a biological sample from a subject, wherein the presence of the biomarker at an increased level over control indicates the presence of GVHD or a risk of GVHD. In additional aspects, the disclosure includes methods of measuring a biomarker in a biological sample from a subject, wherein a decrease in the biomarker level compared to the level prior to treatment for GVHD indicates that the treatment for GVHD is effective. The disclosure also includes methods of measuring a combination of biomarkers in a biological sample from a subject, wherein the presence of the combination of biomarkers at an increased level over control indicates the presence of GVHD or a risk of GVHD. In additional aspects, the disclosure includes methods of measuring a combination of biomarkers in a biological sample from a subject, wherein a decrease in the biomarker level compared to the level prior to treatment for GVHD indicates that the treatment for GVHD is effective

In some aspects, the methods include measuring the level of REG3α protein or nucleic acid in a biological sample. In some aspects, the methods further comprise measuring the level of a second biomarker or a combination of biomarkers with REG3α. Such additional biomarker(s) is selected from the group consisting of: interleukin 2 receptor alpha (IL2Rα), tumor necrosis factor receptor superfamily member 1A (TNFRSF1A or TNFR1), interleukin 8 (IL-8), hepatocyte growth factor (HGF), and elafin. IL2Rα, TNFRSF1A or TNFR1, IL-8, and HGF are biomarkers which have been previously reported to be diagnostic markers of acute GVHD. Elafin is a biomarker which has been previously reported to be a biomarker for GVHD of the skin. The present disclosure includes the use of one or more of these biomarkers in combination with REG3α in methods of diagnosing or predicting GI GVHD.

REG3α, a C-type lectin secreted by Paneth cells, was identified herein as a biomarker specific for lower GI GVHD through an unbiased, in-depth tandem MS-based discovery approach that can quantify proteins at low concentrations. REG proteins act downstream of IL-22 to protect the epithelial barrier function of the intestinal mucosa through the binding of bacterial peptidoglycans. Intestinal stem cells (ISCs) are principal cellular targets of GVHD in the GI tract, where intestinal flora are critical for amplification of GVHD damage. Without being bound by theory, a leading hypothesis is that ISCs are protected by anti-bacterial proteins, such as REG3α, secreted by neighboring Paneth cells into the crypt microenvironment. If death of an ISC eventually manifests itself as denudation of the mucosa, the patchy nature of GVHD histologic damage may be explained as the lack of mucosal regeneration following the dropout of individual ISCs. REG3α reduces the inflammation of human intestinal crypts in vitro, and its administration protects ISCs and prevents GI epithelial damage in vivo, raising interesting therapeutic possibilities for this molecule.

REG3α protein plasma concentrations correlate with disease activity in inflammatory bowel disease, and can distinguish infectious and autoimmune causes of diarrhea: Without being bound by theory, correlation of mucosal denudation (histologic grade 4) with high REG3α concentrations suggests that microscopic breaches in the mucosal epithelial barrier caused by severe GVHD permit REG3α to traverse into the systemic circulation. The tight proximity of Paneth cells with ISCs concentrates their secretory contents in that vicinity, so that mucosal barrier disruption caused by stem cell dropout may preferentially allow Paneth cell secretions, including REG3α, to traverse into the bloodstream. It is hypothesized that plasma levels of REG3α may therefore serve as a surrogate marker for the cumulative area of these breaches to GI mucosal barrier integrity, a parameter impossible to measure by individual tissue biopsies. Without being bound by theory, such an estimate of total damage to the mucosal barrier may also help explain the prognostic value of REG3α with respect to therapy responsiveness and NRM.

In some aspects of the disclosure relating to acute GVHD, ST2 is a biomarker that is used to predict response and survival to therapy for acute GVHD. ST2 is the IL33 receptor, a member of the IL1/Toll-like receptor superfamily. ST2 promotes a Th2-type immune response in diseases, such as arthritis and asthma (Kakkar et al., Nature Reviews Drug Discovery 7: 827-40, 2008). In further aspects, ST2 is a biomarker useful for predicting GVHD as well.

The disclosure includes the use of any one biomarker or combination of biomarkers listed in the table of biomarkers below in any of the disclosed methods, kits, systems, regimens, uses and the like. For example, the disclosure, in various aspects, includes any biomarker or combination of biomarkers as illustrated in columns 1-42 in the Table below.

Table of Biomarkers and Combinations of Biomarkers.

| Column No. | REG3α | Elafin | TNFR1 | IL2Rα | IL-8 | HGF | ST2 |
|---|---|---|---|---|---|---|---|
| 1 | X | | | | | | |
| 2 | X | X | | | | | |
| 3 | X | | X | | | | |
| 4 | X | | | X | | | |
| 5 | X | | | | X | | |
| 6 | X | | | | | X | |
| 7 | X | | | | | | X |
| 8 | | X | | | | | X |
| 9 | | | X | | | | X |
| 10 | | | | X | | | X |
| 11 | | | | | X | | X |
| 12 | | | | | | X | X |
| 13 | | | | | | | X |
| 14 | X | X | X | | | | |
| 15 | X | X | | X | | | |
| 16 | X | X | | | X | | |
| 17 | X | X | | | | X | |
| 18 | X | X | | | | | X |
| 19 | | X | X | | | | X |
| 20 | | | X | X | | | X |
| 21 | | | | X | X | | X |
| 22 | | | | | X | X | X |
| 23 | X | X | X | X | | | |
| 24 | X | X | X | | X | | |
| 25 | X | X | X | | | X | |
| 26 | X | X | X | | | | X |
| 27 | | X | X | X | | | X |
| 28 | | | X | X | X | | X |
| 29 | | | | X | X | X | X |
| 30 | X | X | X | X | X | | |
| 31 | X | X | X | X | | X | |
| 32 | X | X | X | X | | | X |
| 33 | | X | X | X | X | | X |
| 34 | | | X | X | X | X | X |
| 35 | X | X | X | X | X | X | |
| 36 | X | X | X | X | X | | X |
| 37 | X | X | X | X | | X | X |
| 38 | X | X | X | | X | X | X |
| 39 | X | X | | X | X | X | X |
| 40 | X | | X | X | X | X | X |
| 41 | | X | X | X | X | X | X |
| 42 | X | X | X | X | X | X | X |

### Prognostic Value of REG3α Level in the Diagnosis and Treatment of GVHD

In the disclosure, three high-risk parameters each independently correlated with lack of response to treatment and to greater NRM: (1) elevated plasma REG3α concentration, (2) higher clinical stage of GVHD at diagnosis, and (3) grade 4 histologic severity. All three of these values provided important prognostic information prior to the initiation of therapy rather than at the time of maximum grade of GVHD, which by definition includes responsiveness to therapy. This disclosure confirms earlier reports where higher clinical stage of GI GVHD and more severe histology correlated with worse survival.

In the disclosure, the level of a biomarker is measured in a sample from a subject at risk of GVHD and compared to the level of the biomarker in a control. In various aspects, an increased level of biomarker is a level significantly greater than the control level. In various aspects, an increase in the level of the biomarker in a subject is at least or about 25% greater, at least or about 30% greater, at least or about 35% greater, at least or about 40% greater, at least or about 45% greater, at least or about 50% greater, at least or about 55% greater, at least or about 60% greater, at least or about 65% greater, at least or about 70% greater, at least or about 75% greater, at least or about 80% greater, at least or about 85% greater, at least or about 90% greater, at least or about 95% greater, at least or about 100% greater, at least or about 110% greater, at least or about 110% greater, at least or about 120% greater, at least or about 130% greater, at least or about 140% greater, at least or about 150% greater, at least or about 160% greater, at least or about 170% greater, at least or about 180% greater, at least or about 190% greater, at least or about 200% greater, at least or about 220% greater, at least or about 240% greater, at least or about 260% greater, at least or about 280% greater, at least or about 300% greater, at least or about 320% greater, at least or about 340% greater, at least or about 360% greater, at least or about 380% greater, at least or about 400% greater, at least or about 420% greater, at least or about 440% greater, at least or about 460% greater, at least or about 480% greater, at least or about 500% greater, at least or about 520% greater, at least or about 540% greater, at least or about 560% greater, at least or about 580% greater, at least or about 600% greater, at least or about 620% greater, at least or about 640% greater, at least or about 660% greater, at least or about 680% greater, at least or about 700% greater, at least or about 750% greater, at least or about 800% greater, at least or about 850% greater, at least or about 900% greater, at least or about 950% greater, or at least or about 1000% greater than the level of the control. In some aspects, the control level is a median control level.

In additional aspects, an increase in the level of the biomarker in a subject is at least or about ¼ greater, at least or about ½ greater, at least or about 1 time greater, at least or about 2 times greater, at least or about 3 times greater, at least or about 4 times greater, at least or about 5 times greater, at least or about 6 times greater, at least or about 7 times greater, at least or about 8 times greater, at least or about 9 times greater, at least or about 10 times greater, at least or about 12 times greater, at least or about 14 times greater, at least or about 16 times greater, at least or about 18 times greater, or at least or about 20 times greater than the control level.

In other aspects, an increased level of a biomarker in a sample means that the concentration of the biomarker is significantly greater than the control level. Significant differences are calculated according to any statistical analysis method known to one of ordinary skill in the art.

The level of biomarker may be compared to any suitable control level of biomarker representing a standard or normal state. For example, the control level to which the measured level of a biomarker is compared may be an average or median level of biomarker of a population of subjects that are known to not have any risk of GVHD, and, optionally, are matched to the subject in other parameters, such as one or more of the following: age, sex, and the like. In exemplary aspects, the control level is a median control level. Alternatively, the control level to which the measured level of biomarker is compared may be an absolute level.

In some aspects of the invention, a REG3α level indicative of GVHD in a subject ranges from about 10 ng/ml to about 10,000 ng/ml. In particular aspects, the REG3α level is about 10 ng/ml, about 11 ng/ml, about 12 ng/ml, about 13 ng/ml, about 14 ng/ml, about 15 ng/ml, about 16 ng/ml, about 17 ng/ml, about 18 ng/ml, about 19 ng/ml, about 20 ng/ml, about 21 ng/ml, about 22 ng/ml, about 23 ng/ml, about 24 ng/ml, about 25 ng/ml, about 26 ng/ml, about 27 ng/ml, about 28 ng/ml, about 29 ng/ml, about 30 ng/ml, about 31 ng/ml, about 32 ng/ml, about 33 ng/ml, about 34 ng/ml, about 35 ng/ml, about 36 ng/ml, about 37 ng/ml, about 38 ng/ml, about 39 ng/ml, about 40 ng/ml, about 41 ng/ml, about 42 ng/ml, about 43 ng/ml, about 44 ng/ml, about 45 ng/ml, about 46 ng/ml, about 47 ng/ml, about 48 ng/ml, about 49 ng/ml, about 50 ng/ml, about 51 ng/ml, about 52 ng/ml, about 53 ng/ml, about 54 ng/ml, about 55 ng/ml, about 56 ng/ml, about 57 ng/ml, about 58 ng/ml, about 59 ng/ml, about 60 ng/ml, about 61 ng/ml, about 62 ng/ml, about 63 ng/ml, about 64 ng/ml, about 65 ng/ml, about 66 ng/ml, about 67 ng/ml, about 68 ng/ml, about 69 ng/ml, about 70 ng/ml, about 75 ng/ml, about 80 ng/ml, about 85 ng/ml, about 90 ng/ml, about 95 ng/ml, about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, about 200 ng/ml, about 210 ng/ml, about 220 ng/ml, about 230 ng/ml, about 240 ng/ml, about 250 ng/ml, about 260 ng/ml, about 270 ng/ml, about 280 ng/ml, about 290 ng/ml, about 300 ng/ml, about 320 ng/ml, about 340 ng/ml, about 360 ng/ml, about 380 ng/ml, about 400 ng/ml, about 420 ng/ml, about 440 ng/ml, about 460 ng/ml, about 480 ng/ml, about 500 ng/ml, about 520 ng/ml; about 540 ng/ml, about 560 ng/ml, about 580 ng/ml, about 600 ng/ml, about 620 ng/ml, about 640 ng/ml, about 660 ng/ml, about 680 ng/ml, about 700 ng/ml, about 720 ng/ml, about 740 ng/ml, about 760 ng/ml, about 780 ng/ml, about 800 ng/ml, about 820 ng/ml, about 840 ng/ml, about 860 ng/ml, about 880 ng/ml, about 900 ng/ml, about 920 ng/ml, about 940 ng/ml, about 960 ng/ml, about 980 ng/ml, about 1000 ng/ml, about 1500 ng/ml, about 2000 ng/ml, about 2500 ng/ml, about 3000 ng/ml, about 3500 ng/ml, about 4000 ng/ml, about 4500 ng/ml, about 5000 ng/ml, about 5500 ng/ml, about 6000 ng/ml, about 7000 ng/ml, about 8000 ng/ml, about 9000 ng/ml, or about 10,000 ng/ml.

In exemplary aspects, a REG3α level at the onset of diarrhea of 28 ng/ml had a positive predictive value of 84% for GI GVHD; a REG3α level at the onset of diarrhea of 57 ng/ml had a positive predictive value of 92% for GI GVHD; a REG3α level at the onset of diarrhea of 100 ng/ml had a positive predictive value of 95% for GI GVHD; and a REG3α level at the onset of diarrhea of 151 ng/ml had a positive predictive value of 95% for GI GVHD.

In some aspects of the invention, an ST2 level indicative of GVHD in a subject ranges from about 200 pg/ml to about 10,000 pg/ml. In particular aspects, the ST2 level is about 200 pg/ml, about 210 pg/ml, about 220 pg/ml, about 230 pg/ml, about 240 pg/ml, about 250 pg/ml, about 260 pg/ml, about 270 pg/ml, about 280 pg/ml, about 290 pg/ml, about 300 pg/ml, about 320 pg/ml, about 340 pg/ml, about 360 pg/ml, about 380 pg/ml, about 400 pg/ml, about 420 pg/ml, about 440 pg/ml, about 460 pg/ml, about 480 pg/ml, about 500 pg/ml, about 520 pg/ml, about 540 pg/ml, about 560 pg/ml, about 580 pg/ml, about 600 pg/ml, about 620 pg/ml, about 640 pg/ml, about 660 pg/ml, about 680 pg/ml, about 700 pg/ml, about 720 pg/ml, about 740 pg/ml, about 760 pg/ml, about 780 pg/ml, about 800 pg/ml, about 820 pg/ml, about 840 pg/ml, about 860 pg/ml, about 880 pg/ml, about 900 pg/ml, about 920 pg/ml, about 940 pg/ml, about 960 pg/ml, about 980 pg/ml, about 1000 pg/ml, about 1500 pg/ml, about 2000 pg/ml, about 2500 pg/ml, about 3000 pg/ml, about 3500 pg/ml, about 4000 pg/ml, about 4500 pg/ml, about 5000 pg/ml, about 5500 pg/ml, about 6000 pg/ml, about 7000 pg/ml, about 8000 pg/ml, about 9000 pg/ml, or about 10,000 pg/ml.

In exemplary aspects, an ST2 level of about 50% greater than the median control level is indicative of GVHD. In particular aspects of the disclosure, a high or increased level of ST2 at therapy initiation is defined as an ST2 concentration of greater than about 740 pg/mL, and a low or decreased level of ST2 at therapy initiation is defined as an ST2 concentration at therapy initiation of less than or equal to about 740 pg/mL.

In further aspects, the ST2 level indicative of GVHD is dependent upon the treatment that the patient received prior to HCT. For example, at about D14 post-HCT, a high or increased level of ST2 is defined as an ST2 concentration of greater than about 600±200 pg/mL for patients who received chemotherapy-based full intensity conditioning, of greater than about 300±100 pg/mL for patients who received reduced intensity conditioning, and of greater than about 1660±500 pg/mL for patients who received total body irradiation-based full intensity conditioning.

In other aspects of the disclosure, biomarker level is measured after treatment for GVHD. In such aspects, efficacy of treatment is determined by a decrease in biomarker level compared to the level of the biomarker prior to treatment. Methods of measuring biomarker levels are described in the art and herein. In these particular embodiments, therefore, the decreased level of biomarker is at least or about a 10% decrease, at least or about a 15% decrease, at least or about a 20% decrease, at least or about a 25% decrease, at least or about a 30% decrease, at least or about a 35% decrease, at least or about a 40% decrease, at least or about a 45% decrease, at least or about a 50% decrease, at least or about a 55% decrease, at least or about a 60% decrease, at least or about a 65% decrease, at least or about a 70% decrease, at least or about a 75% decrease, at least or about a 80% decrease, at least or about a 85% decrease, at least or about a 90% decrease, at least or about a 95% decrease, at least or about a 100% decrease compared to the level of the biomarker in a subject's biological sample prior to treatment for GVHD.

### Detecting and Measuring Biomarker Level

In various aspects of the disclosure, level of the protein biomarker is detected or quantitatively measured in a biological sample by any suitable means known in the art for quantifying protein including, but not limited to, immunoassay (e.g., ELISA, RIA), immunoturbidimetry, rapid immunodiffusion, laser nephelometry, visual agglutination, quantitative Western blot analysis, multiple reaction monitoring-mass spectrometry (MRM Proteomics), Lowry assay, Bradford assay, BCA assay, and UV spectroscopic assays, such as a UV spectroscopic assay. Alternatively, Northern blotting can be used to compare the levels of mRNA. These processes are described in Sambrook et al., Molecular Cloning: A Laboratory Manual, 3.sup.rd ed. Cold Spring Harbor Laboratory Press, Cold Spring Harbor, N.Y. (2001).

Methods of detecting expression levels are known in the art. For example, ELISA, radioimmunoassays, immunofluorescence, and Western blotting can be used to compare the expression of protein levels. Alternatively, Northern blotting can be used to compare the levels of mRNA. These processes are described in Sambrook et al., Molecular Cloning: A Laboratory Manual, 3.sup.rd ed. Cold Spring Harbor Laboratory Press, Cold Spring Harbor, N.Y. (2001).

Any of these methods may be performed using a nucleic acid (e.g., DNA, mRNA) or protein of a biological sample obtained from the human individual for whom a susceptibility is being determined. The biological sample can be any nucleic acid or protein containing sample obtained from the human individual. For example, the biological sample can be any of the biological samples described herein.

In exemplary aspects, REG3α level is measured by ELISA (MBL International, Woburn, Mass.; Ab-Match Assembly Human PAP1 kit and Ab-Match Universal kit) performed according to the manufacturer's protocol. Samples (diluted 1:10) and standards are run in duplicate. Absorbance is measured with a SpectraMax M2 (Molecular Devices, Sunnyvale, Calif.), and results are calculated with SoftMax Pro v5.4 (Molecular Devices).

In further exemplary aspects, elafin, IL2Rα, HGF, TNFR1, and IL-8 levels are measured by ELISA. In some aspects, such ELISAs are performed in duplicate as previously reported (Paczesny et al., Sci. Transl. Med. 2: 50-7, 2010; Paczesny et al., Blood 113: 273-8, 2009). Details of assay parameters used in various aspects of the disclosure are provided herein in Table 6. Elafin, IL2Rα, HGF, TNFR1, and IL-8 have been described previously as plasma biomarkers for GVHD (Paczesny et al., Biol. Blood Marrow Transplant. 15 (1 Suppl): 33-8, 2008).

Specificity and sensitivity are best represented by a Receiver Operating Characteristic (ROC) curve which is a plot of the false positive rate on the x axis and true positive rate on the y axis for every possible level of a marker. A perfect test would have a ROC curve that is a right angle demonstrating 100% of true positives and no false positives. In this case, the corresponding Area Under the Curve (AUC) equals 1. A random test has an AUC of 0.5, meaning that there is one false positive for every true positive. A biomarker panel, in various aspects, includes several biomarkers that together are diagnostic or predictive.

In some aspects of the disclosure, the level of an mRNA biomarker is detected or quantitatively measured in a biological sample by any suitable means known in the art for quantifying mRNA including, but not limited to, Northern blotting, RT-qPCR, direct digital quantification, and serial analysis of gene expression (SAGE).

### Methods of the Disclosure

In some embodiments, methods are provided for detecting GVHD in a subject, comprising measuring a level of a biomarker in a biological sample isolated from the subject, wherein the biomarker is REG3α, and wherein an increased level of the biomarker present in the biological sample compared to a control level indicates GVHD in the subject.

In some embodiments, methods are provided for predicting GVHD in a subject, comprising measuring a level of a biomarker in a biological sample isolated from the subject, wherein the biomarker is REG3α, and wherein an increased level of the biomarker present in the biological sample compared to a control level predicts GVHD in the subject.

In additional embodiments, methods are provided for treating GVHD in a subject suffering from GVHD comprising the steps of identifying the subject at risk of suffering from GVHD, measuring a level of a biomarker in a biological sample isolated from the subject, wherein the biomarker is REG3α, and wherein an increased level of the biomarker present in the biological sample compared to a control level indicates GVHD in the subject; and administering an effective amount of a treatment for GVHD to the subject.

In further embodiments, methods are provided for determining efficacy of a therapeutic agent in treating a subject suffering from GVHD comprising the steps of administering to the subject the therapeutic agent, and measuring a level of biomarker in a biological sample obtained from the subject, wherein a decrease in the level of biomarker relative to the level prior to administration of the therapeutic agent, indicates that the therapeutic agent is effective for treating GVHD in the subject.

The methods optionally comprise additional steps, as noted herein, or as otherwise appreciated by the ordinarily skilled artisan. For example, the methods of the disclosure optionally comprise, unless noted otherwise, one or more of the following steps: (i) determining whether the subject is suffering from GVHD, (ii) determining whether the subject is at risk from suffering from GVHD, (iii) measuring the level of one or more biomarkers in a biological sample obtained from the subject, and, if necessary (iv) administering to the subject an effective amount of a treatment or prophylaxis for GVHD. In an additional example, the methods of the disclosure optionally comprise, unless noted otherwise, one or more of the following steps: (i) determining whether the subject is suffering from GVHD, (ii) measuring a level of one or more biomarkers in a biological sample obtained from the subject, (iii) administering a treatment for GVHD, (iv) measuring the level of one or more biomarkers in a biological sample obtained from the subject after treatment, and (v) comparing the level of the biomarkers before and after treatment, wherein a decrease in the biomarker level after treatment indicates that the treatment is effective in GVHD. The methods optionally comprise measuring the levels of additional markers of GVHD.

In cases in which a method comprises combination of steps, each and every combination or sub-combination of the steps is encompassed within the scope of the disclosure, unless otherwise noted herein.

In regard to any of the methods provided, the steps of the method may occur simultaneously or sequentially. When the steps of the method occur sequentially, the steps may occur in any order, unless noted otherwise.

### Kits

As an additional aspect, the disclosure includes kits which comprise reagents packaged in a manner which facilitates their use for measuring a biomarker in a biological sample from a subject suspected of having GVHD. In some variations, such reagents are packaged together. In some variations, the kit further includes an analysis tool for evaluating risk of a subject developing GVHD from a measurement of the biomarker from a biological sample from the subject.

In one embodiment, the disclosure pertains to a kit for assaying a sample from a subject to detect a susceptibility to GVHD in the subject, wherein the kit comprises reagents necessary for selectively detecting a biomarker or a combination of biomarkers in the subject. In certain embodiments, the biomarker is REG3α or ST2. In additional embodiments, the combination of biomarkers comprises REG3α or ST2. In particular embodiments, the combination of biomarkers comprises REG3α and further comprises any of elafin, tumor necrosis factor receptor 1 (TNFR1), interleukin-2 receptor alpha chain (IL2Rα), interleukin 8 (IL-8), and hepatocyte growth factor (HGF). In other embodiments, the combination of biomarkers comprises ST2 and further comprises any of elafin, tumor necrosis factor receptor 1 (TNFR1), interleukin-2 receptor alpha chain (IL2Rα), interleukin 8 (IL-8), REG3α and hepatocyte growth factor (HGF). In more particular embodiments, the combination of biomarkers comprises REG3α; elafin, TNFR1, and IL2Rα. In even more particular embodiments, the combination of biomarkers comprises REG3α, IL2Rα, and elafin. In exemplary embodiments, the kit comprises antibodies for detecting the biomarkers or combinations of biomarkers.

In a further aspect of the present invention, a pharmaceutical pack (kit) is provided, the pack comprising a therapeutic agent and a set of instructions for administration of the therapeutic agent to a subject diagnostically tested for risk of GVHD. The therapeutic agent can be any of the therapeutic agents described herein for treating GVHD.

In some embodiments, the kit further comprises a set of instructions for using the reagents comprising the kit. In certain embodiments, the kit further comprises a collection of data comprising correlation data between the biomarker level and the susceptibility to GVHD.

In a specific embodiment, the kits of the disclosure each contain an apparatus for collecting a biological sample from a subject and reagents for measuring the level of biomarker in a biological sample. In a further aspect, the kit comprises optional instructions included in the package that describes use of the reagents packaged in the kit for practicing the method.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more Controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, and the like.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

Thus, another aspect of the disclosure is a system that is capable of carrying out a part or all of a method of the disclosure, or carrying out a variation of a method of the disclosure as described herein in greater detail. Exemplary systems include, as one or more components, computing systems, environments, and/or configurations that may be suitable for use with the methods and include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. In some variations, a system of the disclosure includes one or more machines used for analysis of biological material (e.g., genetic material), as described herein. In some variations, this analysis of the biological material involves a chemical analysis and/or a nucleic acid amplification.

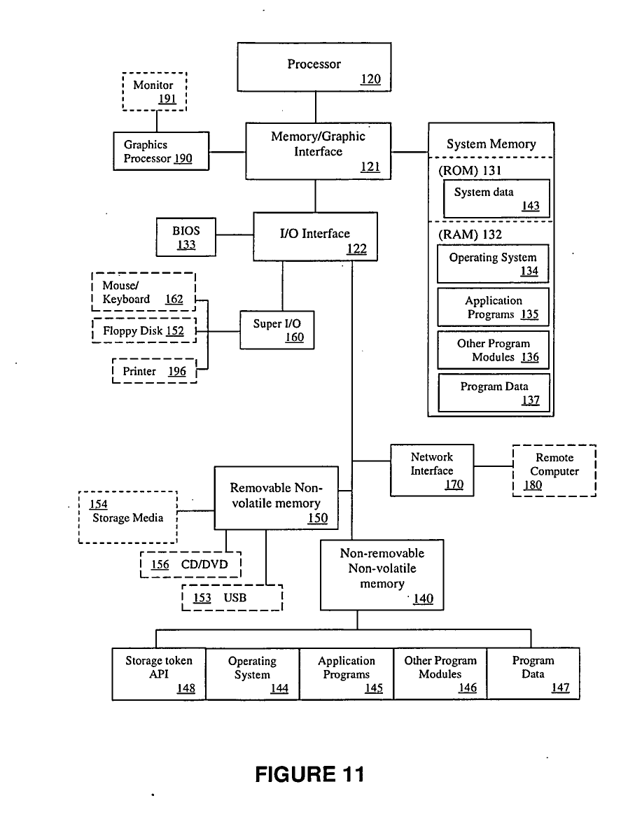

With reference to FIG. 11, an exemplary system of the disclosure, which may be used to implement one or more steps of methods of the disclosure, includes a computing device in the form of a computer 110. Components shown in dashed outline are not technically part of the computer 110, but are used to illustrate the exemplary embodiment of FIG. 11. Components of computer 110 may include, but are not limited to, a processor 120, a system memory 130, a memory/graphics interface 121, also known as a Northbridge chip, and an I/O interface 122, also known as a Southbridge chip. The system memory 130 and a graphics processor 190 may be coupled to the memory/graphics interface 121. A monitor 191 or other, graphic output device may be coupled to the graphics processor 190.

A series of system busses may couple various system components including a high speed system bus 123 between the processor 120, the memory/graphics interface 121 and the I/O interface 122, a front-side bus 124 between the memory/graphics interface 121 and the system memory 130, and an advanced graphics processing (AGP) bus 125 between the memory/graphics interface 121 and the graphics processor 190. The system bus 123 may be any of several types of bus structures including, by way of example, and not limitation, such architectures include Industry Standard Architecture (ISA) bus, Micro Channel Architecture (MCA) bus and Enhanced ISA (EISA) bus. As system architectures evolve, other bus architectures and chip sets may be used but often generally follow this pattern. For example, companies such as Intel and AMD support the Intel Hub Architecture (IHA) and the Hypertransport™ architecture, respectively.

The computer 110 typically includes a variety of computer-readable media. Computer-readable media are any available media that can be accessed by computer 110 and includes both volatile and nonvolatile media, removable and non-removable media. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or, other memory technology, CD-ROM, digital versatile disks (DVD) or other optical disk storage, magnetic cassettes, magnetic tape, magnetic disk storage or other magnetic storage devices, or any other physical medium which can be used to store the desired information and which can accessed by computer 110.

The system memory 130 includes computer storage media in the form of volatile and/or nonvolatile memory such as read only memory (ROM) 131 and random access memory (RAM) 132. The system ROM 131 may contain permanent system data 143, such as identifying and manufacturing information. In some embodiments, a basic input/output system (BIOS) may also be stored in system ROM 131. RAM 132 typically contains data and/or program modules that are immediately accessible to and/or presently being operated on by processor 120. By way of example, and not limitation, FIG. 11 illustrates operating system 134, application programs 135, other program modules 136, and program data 137.

The I/O interface 122 may couple the system bus 123 with a number of other busses 126, 127 and 128 that couple a variety of internal and external devices to the computer 110. A serial peripheral interface (SPI) bus 126 may connect to a basic input/output system (BIOS) memory 133 containing the basic routines that help to transfer information between elements within computer 110, such as during start-up.

A super input/output chip 160 may be used to connect to a number of ‘legacy’ peripherals, such as floppy disk 152, keyboard/mouse 162, and printer 196, as examples. The super I/O chip 160 may be connected to the I/O interface 122 with a bus 127, such as a low pin count (LPC) bus, in some embodiments. Various embodiments of the super I/O chip 160 are widely available in the commercial marketplace.

In one embodiment, bus 128 may be a Peripheral Component Interconnect (PCI) bus, or a variation thereof, may be used to connect higher speed peripherals to the I/O interface 122. A PCI bus may also be known as a Mezzanine bus. Variations of the PCI bus include the Peripheral Component Interconnect-Express (PCI-E) and the Peripheral Component Interconnect-Extended (PCI-X) busses, the former having a serial interface and the latter being a backward compatible parallel interface. In other embodiments, bus 128 may be an advanced technology attachment (ATA) bus, in the form of a serial ATA bus (SATA) or parallel ATA (PATA).

The computer. 110 may also include other removable/non-removable, volatile/nonvolatile computer storage media. By way of example only, FIG. 11 illustrates a hard disk drive 140 that reads from or writes to non-removable, nonvolatile magnetic media. The hard disk drive 140 may be a conventional hard disk drive.

Removable media, such as a universal serial bus (USB) memory 153, firewire (IEEE 1394), or CD/DVD drive 156 may be connected to the PCI bus 128 directly or through an interface 150. A storage media 154 may couple through interface 150. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

The drives and their associated computer storage media discussed above and illustrated in FIG. 11, provide storage of computer readable instructions, data structures, program modules and other data for the computer 110. In FIG. 11, for example, hard disk drive 140 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a mouse/keyboard 162 or other input device combination. Other input devices (not shown) may include a microphone, joystick, game pad, satellite dish, scanner, or the like. These and other input devices are often connected to the processor 120 through one of the I/O interface busses, such as the SPI 126, the LPC 127, or the PCI 128, but other busses may be used. In some embodiments, other devices may be coupled to parallel ports, infrared interfaces, game ports, and the like (not depicted), via the super I/O chip 160.

The computer 110 may operate in a networked environment using logical connections to one or more remote computers, such as a remote computer 180 via a network interface controller (NIC) 170. The remote computer 180 may be a personal computer, a server, a router, a network PC, a peer device or other common network node, and typically includes many or all of the elements described above relative to the computer 110. The logical connection between the NIC 170 and the remote computer 180 depicted in FIG. 11 may include a local area network (LAN), a wide area network (WAN), or both, but may also include other networks. Such networking environments are commonplace in offices, enterprise-wide computer networks, intranets, and the Internet. The remote computer 180 may also represent a web server supporting interactive sessions with the computer 110; or in the specific case of location-based applications may be a location server or an application server.

In some embodiments, the network interface may use a modem (not depicted) when a broadband connection is not available or is not used. It will be appreciated that the network connection shown is exemplary and other means of establishing a communications link between the computers may be used.

In some variations, the disclosure provides a system for identifying susceptibility to GVHD in a human subject. For example, in one variation, the system includes tools for performing at least one step, preferably two or more steps, and in some aspects all steps of a method of the disclosure, where the tools are operably linked to each other. Operable linkage describes a linkage through which components can function with each other to perform their purpose.

In some variations, a system of the disclosure is a system for identifying susceptibility of developing GVHD in a subject, the system comprising: at least one processor; at least one computer-readable medium; a susceptibility database operatively coupled to a computer-readable medium of the system and containing population information correlating protein level of a biomarker or a combination of biomarkers in a subject to susceptibility to developing GVHD in a population of humans, wherein the biomarker or the combination of biomarkers is selected from the group consisting of REG3α and ST2; a measurement tool that receives an input about the subject and generates information from the input about the protein level of the biomarker or the combination of biomarkers in the subject, wherein an elevated protein level of the biomarker or the combination of biomarkers is associated with increased susceptibility to GVHD; and an analysis tool that is operatively coupled to the susceptibility database and the measurement tool is stored on a computer-readable medium of the system, is adapted to be executed on a processor of the system, to compare the information about the subject with the population information in the susceptibility database and generate a conclusion with respect to susceptibility of developing GVHD for the subject.

In other variations, a system of the disclosure further comprises a susceptibility database, wherein the susceptibility database further comprises population information correlating a clinical parameter or a combination of clinical parameters in the subject to susceptibility to developing GVHD in a population of humans, wherein the clinical parameter or combination of clinical parameters is selected from the group consisting of: age of the subject; whether the subject received a bone marrow transplantation or a peripheral blood stem cell transplantation, whether all human leukocyte antigens were matched or mismatched in the transplant, whether subject received previous treatment with tacrolimus and methotrexate, whether subject received high toxicity conditioning without total body irradiation; whether subject received high toxicity conditioning with or without total body irradiation to susceptibility to developing GVHD in a population of humans; and wherein the measurement tool further generates information from the input about the clinical parameter or combination of clinical parameters in the subject, and the impact of the presence or absence of the clinical parameter or combination of clinical parameters on identifying susceptibility of developing GVHD.

Exemplary processors (processing units) include all variety of microprocessors and other processing units used in computing devices. Exemplary computer-readable media are described above. When two or more components of the system involve a processor or a computer-readable medium, the system generally can be created where a single processor and/or computer readable medium is dedicated to a single component of the system; or where two or more functions share a single processor and/or share a single computer readable medium, such that the system contains as few as one processor and/or one computer readable medium. In some variations, it is advantageous to use multiple processors or media, for example, where it is convenient to have components of the system at different locations. For instance, some components of a system may be located at a testing laboratory dedicated to laboratory or data analysis, whereas other components, including components (optional) for supplying input information or obtaining an output communication, may be located at a medical treatment or counseling facility (e.g., doctor's office, health clinic, HMO, pharmacist, geneticist, hospital) and/or at the home or business of the human subject (patient) for whom the testing service is performed.

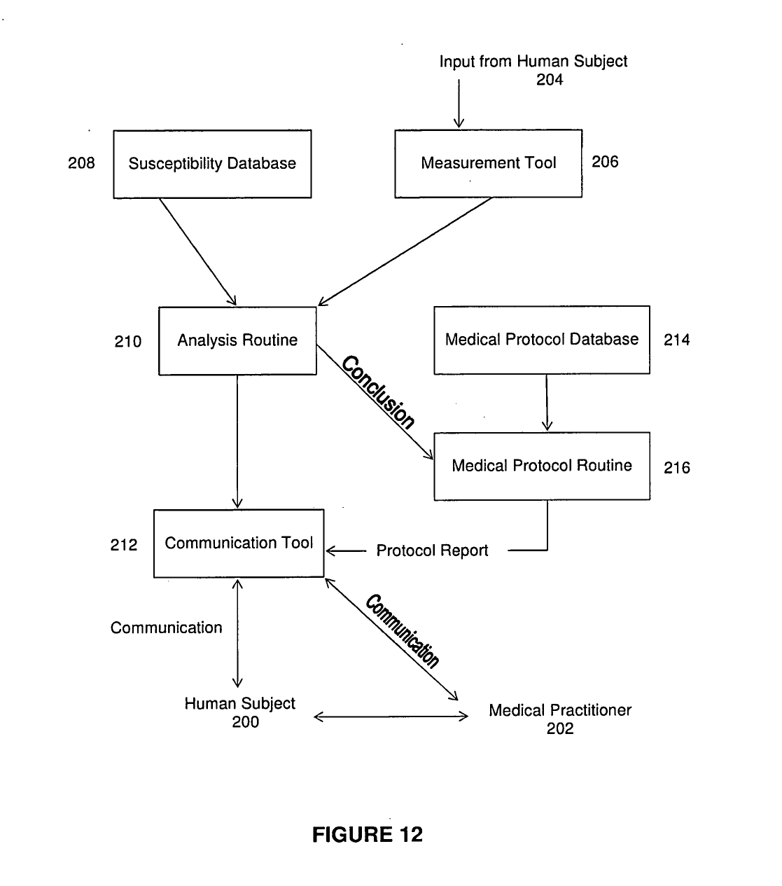

Referring to FIG. 12, an exemplary system includes a susceptibility database 208 that is operatively coupled to a computer-readable medium of the system and that contains population information correlating the level of biomarker or combination of biomarkers and susceptibility to a GVHD in a population of subjects.

In a simple-variation, the susceptibility database contains 208 data relating to the frequency that a particular level of biomarker has been observed in a population of human subjects with GVHD and a population of human subjects free of GVHD. Such data provides an indication as to the risk or probability of developing GVHD for a human subject that is identified as being at risk of developing GVHD. In another variation, the susceptibility database includes similar data with respect to a combination of biomarkers. In still another variation, the susceptibility database includes additional quantitative personal, medical, or genetic information about the subjects in the database diagnosed with GVHD or free of GVHD. Such information includes, but is not limited to, information about parameters and/or clinical parameters, such as age, type of transplantation (e.g., bone marrow transplantation or a peripheral blood stem cell transplantation), whether all human leukocyte antigens (HLA) were matched or mismatched in the transplant, whether the subject received previous treatment with tacrolimus and methotrexate, whether the subject received high toxicity conditioning without total body irradiation, and whether the subject received high toxicity conditioning with or without total body irradiation, and the impact of any of these parameters on susceptibility to GVHD. Additional information includes subject's sex, ethnicity, race, medical history, weight, diabetes status, blood pressure, family history of cancer, smoking history, alcohol use and the impact of any of these parameters on susceptibility to GVHD. These more robust susceptibility databases can be used by an analysis routine 210 to calculate a combined score with respect to susceptibility or risk for developing GVHD.

In addition to the susceptibility database 208, the system further includes a measurement tool 206 programmed to receive an input 204 from or about the human subject and generate an output that contains information about the level of biomarker or combination of biomarkers and, optionally about the presence or absence of various clinical parameters described herein. (The input 204 is not part of the system per se but is illustrated in the schematic FIG. 12.) Thus, the input 204 will contain a specimen or contain data about the level of biomarker or combination of biomarkers and, optionally data about the presence or absence of various clinical parameters, which can be directly read, or analytically determined. In a simple variation, the input contains annotated information about biomarker levels in a human subject, in which case no further processing by the measurement tool 206 is required, except possibly transformation of the relevant information about the level of biomarker or combination of biomarkers and, optionally about the presence or absence of various clinical parameters, into a format compatible for use by the analysis routine 210 of the system.

In another variation, the input 204 from the human subject contains data that is unannotated or insufficiently annotated with respect to biomarker level, requiring analysis by the measurement tool 206. For example, the input can be a biological sample, including blood, plasma, or isolated protein or nucleic acid from the biological sample. In such variations of the disclosure, the measurement tool 206 comprises a tool, preferably stored on a computer-readable medium of the system and adapted to be executed on a processor of the system, to receive a data input about a subject and determine information about the level of biomarker or combination of biomarkers in a human subject from the data. For example, the measurement tool 206 contains instructions, preferably executable on a processor of the system, for analyzing the unannotated input data and determining the expression level of biomarker of interest in the human subject. Where the input data is a biological sample comprising protein, and the measurement tool optionally comprises a protein measurement tool stored on a computer readable medium of the system and executable by a processor of the system with instructions for determining the level of biomarker from the protein sample information.

In yet another variation, the input 204 from the human subject comprises a biological sample, such as a fluid (e.g., blood) or tissue sample, which contains genetic material or protein material that can be analyzed to determine the expression level of biomarker. In this variation, an exemplary measurement tool 206 includes laboratory equipment for processing and analyzing the sample to determine the expression level of biomarker in the human subject. For instance, in one variation, the measurement tool includes: an immunoassay containing a plurality of antibodies, attached to a solid support; a detector for measuring interaction between protein obtained from the biological sample and one or more antibodies attached to a solid support to generate detection data; and an analysis tool stored on a computer-readable medium of the system and adapted to be executed on a processor of the system, to determine the expression level of biomarker of interest based on the detection data.

To provide another example, in some variations the measurement tool 206 includes: a nucleotide sequencer (e.g., an automated DNA sequencer) that is capable of determining nucleotide sequence information from nucleic acid obtained from or amplified from the biological sample; and an analysis tool stored on a computer-readable medium of the system and adapted to be executed on a processor of the system, to determine the presence or absence of the expression level of biomarker based on the nucleotide sequence information.

In some variations, the measurement tool 206 further includes additional equipment and/or chemical reagents for processing the biological sample to purify protein or nucleic acid and/or amplify nucleic acid of the human subject for further analysis. In some aspects, further analysis of nucleic acid is carried out using a sequencer, gene chip, or other analytical equipment.

The exemplary system further includes an analysis tool or routine 210 that: is operatively coupled to the susceptibility database 208 and operatively coupled to the measurement tool 206, is stored on a computer-readable medium of the system, is adapted to be executed on a processor of the system to compare the information about the human subject with the population information in the susceptibility database 208 and generate a conclusion with respect to susceptibility to GVHD for the human subject. In simple terms, the analysis tool 210 looks at the expression level of biomarker obtained by the measurement tool 206 for the human subject, and compares this information to the susceptibility database 208, to determine a susceptibility to GVHD for the subject. The susceptibility can be based on the single parameter (the expression level of a biomarker), multiple parameters (the expression level of a combination of biomarkers), or can involve a calculation based on other data, as described above, that is collected and included as part of the input 204 from the human subject, and that also is stored in the susceptibility database 208 with respect to a population of other humans. Generally speaking, each parameter of interest is weighted to provide a conclusion with respect to susceptibility to GVHD. Such a conclusion is expressed in any statistically useful form, for example, as a score, risk score, or a probability for the subject developing GVHD.

In some variations of the disclosure, the system as just described further includes a communication tool 212. For example, the communication tool is operatively connected to the analysis routine 210 and comprises a routine stored on a computer-readable medium of the system and adapted to be executed on a processor of the system, to: generate a communication containing the conclusion; and to transmit the communication to the human subject 200 or the medical practitioner 202, and/or enable the subject or medical practitioner to access the communication. (The subject and medical practitioner are depicted in the schematic FIG. 12, but are not part of the system per se, though they may be considered users of the system. The communication tool 212 provides an interface for communicating to the subject, or to a medical practitioner for the subject (e.g., doctor, nurse, genetic counselor), the conclusion generated by the analysis tool 210 with respect to susceptibility to GVHD for the subject. Usually, if the communication is obtained by or delivered to the medical practitioner 202, the medical practitioner will share the communication with the human subject 200 and/or counsel the human subject about the medical significance of the communication. In some variations, the communication is provided in a tangible form, such as a printed report or report stored on a computer readable medium such as a flash drive or optical disk. In some variations, the communication is provided electronically with an output that is visible on a video display or audio output (e.g., speaker). In some variations, the communication is transmitted to the subject or the medical practitioner, e.g., electronically or through the mail. In some variations, the system is designed to permit the subject or medical practitioner to access the communication, e.g., by telephone or computer. For instance, the system may include software residing on a memory and executed by a processor of a computer used by the human subject or the medical practitioner, with which the subject or practitioner can access the communication, preferably securely, over the internet or other network connection. In some variations of the system, this computer will be located remotely from other components of the system, e.g., at a location of the human subject's or medical practitioner's choosing.

In some variations of the disclosure, the system as described (including embodiments with or without the communication tool) further includes components that add a treatment or prophylaxis utility to the system. For instance, value is added to a determination of susceptibility to GVHD when a medical practitioner can prescribe or administer a standard of care that can reduce susceptibility to GVHD; and/or delay onset of GVHD; and/or increase the likelihood of detecting GVHD at an early stage, to facilitate early treatment of GVHD.

For example, in some variations, the system-further includes a medical protocol database 214 operatively connected to a computer-readable medium of the system and containing information correlating the level of biomarker or combination of biomarkers of interest and medical protocols for human subjects at risk for GVHD. Such medical protocols include any variety of treatments for GVHD. The information correlating a biomarker level with protocols could include, for example, information about the success with which GVHD is avoided, or success with which GVHD is detected early and treated, if a subject has certain biomarker level and follows a treatment protocol.

A system of this embodiment further includes a medical protocol tool or routine 216, operatively connected to the medical protocol database 214 and to the analysis tool or routine 210. The medical protocol tool or routine 216 preferably is stored on a computer-readable medium of the system, and adapted to be executed on a processor of the system, to: (i) compare (or correlate) the conclusion that is obtained from the analysis routine 210 (with respect to susceptibility to GVHD for the subject) and the medical protocol database 214, and (ii) generate a protocol report with respect to the probability that one or more medical protocols in the medical protocol database will achieve one or more of the goals of reducing susceptibility to GVHD; delaying onset of GVHD; and increasing the likelihood of detecting GVHD at an early stage to facilitate early treatment. The probability can be based on empirical evidence collected from a population of humans and expressed either in absolute terms (e.g., compared to making no intervention), or expressed in relative terms, to highlight the comparative or additive benefits of two or more protocols.

Some variations of the system just described include the communication tool 212. In some examples, the communication tool generates a communication that includes the protocol report in addition to, or instead of, the conclusion with respect to susceptibility.

Information about biomarker level alone, or in combination with clinical parameter information, not only can provide useful information about identifying or quantifying susceptibility to GVHD; it can also provide useful information about possible causative factors for a human subject identified with GVHD, and useful information about therapies for GVHD in a subject suffering from GVHD. In some variations, systems of the disclosure are useful for these purposes.

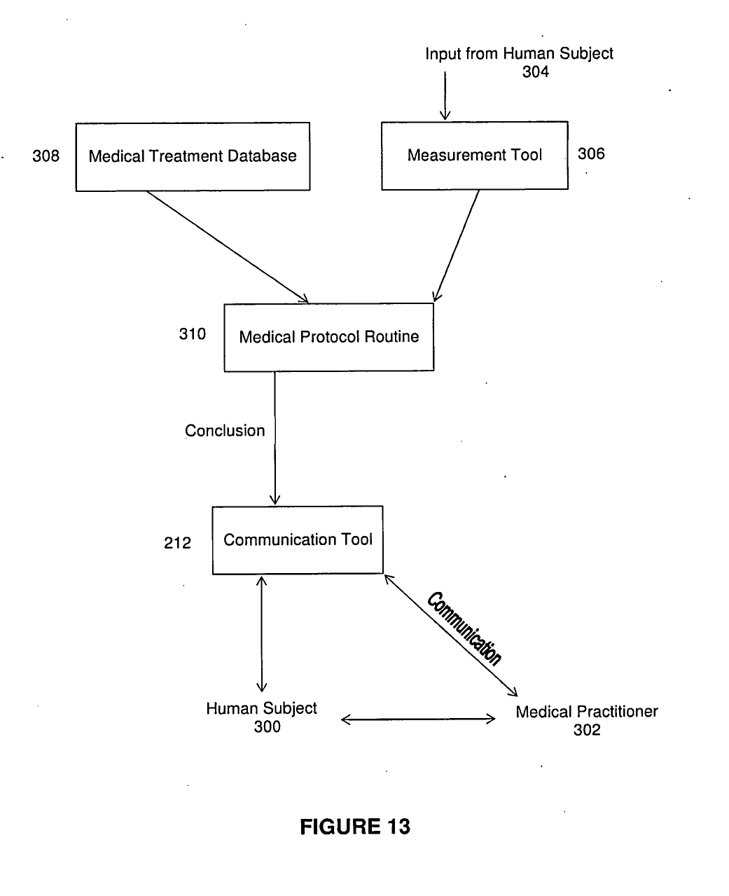

For instance, in some variations the disclosure is a system for assessing or selecting a treatment protocol for a subject diagnosed with GVHD. An exemplary system, schematically depicted in FIG. 13, comprises: (a) at least one processor; (b) at least one computer-readable medium; (c) a medical treatment database 308 operatively connected to a computer-readable medium of the system and containing information correlating the level of biomarker or combination of biomarkers and efficacy of treatment regimens for GVHD; (d) a measurement tool 306 to receive an input (304, depicted in FIG. 13 but not part of the system per se) about the subject and generate information from the input 304 about the level of biomarker or combination of biomarkers in a human subject diagnosed with GVHD; and (e) a medical protocol routine or tool 310 operatively coupled to the medical treatment database 308 and the measurement tool 306, stored on a computer-readable medium of the system, and adapted to be, executed on a processor of the system, to compare the information with respect to the level of biomarker or combination of biomarkers for the subject and the medical treatment database, and generate a conclusion with respect to at least one of: (i) probability that one or more medical treatments will be efficacious for treatment of GVHD for the subject; and (ii) which of two or more medical treatments for GVHD will be more efficacious for the subject.

Preferably, such a system further includes a communication tool 312 operatively connected to the medical protocol tool or routine 310 for communicating the conclusion to the subject 300, or to a medical practitioner for the subject 302 (both depicted in the schematic of FIG. 13, but not part of the system per se). An exemplary communication tool comprises a routine stored on a computer-readable medium of the system and adapted to be executed on a processor of the system, to generate a communication containing the conclusion; and transmit the communication to the subject or the medical practitioner, or enable the subject or medical practitioner to access the communication.

Each publication, patent application, patent, and other reference cited herein is incorporated by reference in its entirety to the extent that it is not inconsistent with the present disclosure.

Recitation of ranges of values herein are merely intended to serve as a shorthand method for referring individually to each separate value falling within the range and each endpoint, unless otherwise indicated herein, and each separate value and endpoint is incorporated into the specification as if it were individually recited herein.

All methods described herein are performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

Additional aspects and details of the disclosure will be apparent from the following examples, which are intended to be illustrative rather than limiting.

### Example 1

### Materials and Methods

### Proteomics Analysis

Methods for sample preparation, protein fractionation, mass spectrometry (MS) analysis, protein identification, and quantitative analysis of protein concentrations during the intact protein analysis system (IPAS) have been previously reported (Faca et al., J. Proteome Res. 5: 2009-18, 2006; Faca et al., J. Proteome Res. 6: 3558-65, 2007; Paczesny et al., Sci. Transl. Med. 2: 50-7, 2010).

### Subjects and Samples

In a first trial (at the University of Michigan), heparinized blood samples were collected weekly for four weeks after allogeneic HCT, then monthly for two months, and also at the time of key clinical events, including the development of symptoms consistent with GVHD, e.g., the onset of diarrhea. Plasma samples were collected prospectively per institutional guidelines. GVHD assessments, sample processing and storage were performed as previously described (Przepiorka et al., Bone Marrow Transpl. 15: 825-8, 1995; Paczesny et al., Sci. Transl. Med. 2: 50-7, 2010).

In an additional trial (at Regensburg, Germany and Kyushu, Japan), serum samples were collected weekly and at the onset of GVHD symptoms. Samples were prepared, frozen and stored per institutional guidelines. Samples were shipped and received frozen on dry ice; no sample was thawed more than twice before analysis. REG3α concentrations were stable in samples frozen for at least five years. REG3α concentrations from the plasma and serum of 12 paired, healthy donors were similar (mean±SEM: 20±3 versus 24±3 ng/ml, respectively).

All subjects received pharmacologic GVHD prophylaxis with at least two agents, including a calcineurin inhibitor. No donor grafts were depleted of T cells. All subjects with available samples were analyzed, including subjects who developed other complications of HCT, such as sinusoidal obstruction syndrome (SOS), idiopathic pneumonia syndrome (IPS) and sepsis/bacteremia. Subjects were excluded from analysis only if a plasma sample at the time of GVHD onset was not available, or if methylprednisolone >1 mg/kg (or equivalent) had been administered for more than 48 hours at the time of sample acquisition. One sample was analyzed per subject.

A discovery set consisted of plasma samples from ten HCT subjects at the onset of biopsy-proven GI GVHD (clinical stage 1-3) and ten HCT subjects who never developed GVHD and who were matched for key transplant characteristics (Table 1). Subject samples in the discovery set were not included in the validation set.

TABLE 1
Patient characteristics of the discovery set.

| | GI GVHD | No GVHD | |
|---|---|---|---|
| Total N = 20 | N = 10 | N = 10 | p-value |
| Age (years) | | | >0.9 |
| Median | 52 | 50 | |
| (range) | (27-60) | (34-64) | |
| Disease (%) | | | >0.9 |
| Malignant | 100% | 100% | |
| | (N = 10) | (N = 10) | |
| Other | 0% | 0% | |
| | (N = 0) | (N = 0) | |
| Disease status at | | | >0.9 |
| transplant* (%) | | | |
| Other/low/ | 60% | 50% | |
| Intermediate risk | (N = 6) | (N = 5) | |
| High risk | 40% | 50% | |
| | (N = 4) | (N = 5) | |
| Donor type (%) | | | >0.9 |
| Related donor | 70% | 70% | |
| | (N = 7) | (N = 7) | |
| Unrelated donor | 30% | 30% | |
| | (N = 3) | (N = 3) | |
| Donor match (%) | | | >0.9 |
| Matched donor | 100% | 90% | |
| | (N = 10) | (N = 9) | |
| Mismatched donor | 0% | 10% | |
| | (N = 0) | (N = 1) | |
| Conditioning regimen | | | >0.9 |
| intensity (%) | | | |
| High intensity | 100% | 90% | |
| | (N = 10) | (N = 9) | |
| Moderate intensity | 0% | 10% | |
| | (N = 0) | (N = 1) | |
| Grade of GVHD at | | | |
| onset (%) | | | |
| 0 | 0% | 100% | |
| | (N = 0) | (N = 10) | |
| I | 0% | 0% | |
| | (N = 0) | (N = 0) | |
| II | 10% | 0% | |
| | (N = 1) | (N = 0) | |
| Isolated Upper GI GVHD | 10% | 0% | |
| | (N = 1) | (N = 0) | |
| Lower GI GVHD | 0% | 0% | |
| | (N = 0) | (N = 0) | |
| III-IV | 90% | 0% | |
| | (N = 9) | (N = 0) | |
| GI Stage 2 | 50% | 0% | |
| | (N = 5) | (N = 0) | |
| GI Stage 3 | 40% | 0% | |
| | (N = 4) | (N = 0) | |
| GI Stage 4 | 0% | 0% | |
| | (N = 0) | (N = 0) | |
| Day after HCT | | | 0.7 |
| Median | 26 | 27 | |
| (range) | (7-63) | (14-70) | |

*High risk of disease status at HCT is according to Center for International Blood and Marrow Transplant Research (CIBMTR) guidelines.

A validation set from the University of Michigan consisted of four groups: (1) subjects with newly diagnosed GVHD involving the GI tract (with or without other organ involvement) (GI GVHD); (2) subjects at similar time points who never developed GVHD symptoms (no GVHD); (3) subjects with GI distress that was inconsistent with GVHD, either by clinical or histologic criteria (non-GVHD enteritis); and (4) subjects who presented with isolated skin GVHD (skin GVHD). Patient, i.e. human subject, numbers and characteristics are shown in Table 2. Enteritis was determined to be inconsistent with GVHD on clinical grounds by documentation of infected stool and by resolution of symptoms without steroid treatment. The etiologies of non-GVHD enteritis are listed in Table 3.

TABLE 2
Patient characteristics of the University of Michigan validation set.

| | | | Non- | | |
|---|---|---|---|---|---|
| | GI | No | GVHD | Skin | |
| | GVHD^{†,‡} | GVHD | Enteritis^{§} | GVHD | p- |
| Total N = 871 | N = 167 | N = 362 | N = 52 | N = 290 | Value |
| Age (years) | | | | | 0.003 |
| Median | 50 | 46 | 48 | 49 | |
| (range) | (0-67) | (0-68) | (3-66) | (0-70) | |
| Disease (%) | | | | | 0.002 |
| Malignant | 99% | 92% | 96% | 97% | |
| | (N = 165) | (N = 334) | (N = 50) | (N = 282) | |
| Other | 1% | 8% | 4% | 3% | |
| | (N = 2) | (N = 28) | (N = 2) | (N = 8) | |
| Disease status at | | | | | 0.63 |
| transplant* (%) | | | | | |
| Other/low/ | 64% | 69% | 68% | 68% | |
| Intermediate risk | (N = 105) | (N = 232) | (N = 34) | (N = 192) | |
| High risk | 36% | 31% | 32% | 32% | |
| | (N = 60) | (N = 102) | (N = 16) | (N = 90) | |
| Donor type (%) | | | | | <0.001 |
| Related donor | 45% | 64% | 54% | 40% | |
| | (N = 75) | (N = 233) | (N = 28) | (N = 115) | |
| Unrelated donor | 55% | 36% | 46% | 60% | |
| | (N = 92) | (N = 129) | (N = 24) | (N = 175) | |
| Donor match (%) | | | | | <0.001 |
| Matched donor | 70% | 90% | 92% | 73% | |
| | (N = 117) | N = (325) | (N = 48) | (N = 212) | |
| Mismatched | 30% | 10% | 8% | 27% | |
| donor | (N = 50) | (N = 37) | (N = 4) | (N = 78) | |
| Conditioning | | | | | 0.06 |
| regimen | | | | | |
| intensity (%) | | | | | |
| High intensity | 57% | 67% | 63% | 57% | |
| | (N = 95) | (N = 243) | (N = 33) | (N = 165) | |
| Moderate | 43% | 33% | 37% | 43% | |
| intensity | (N = 72) | (N = 119) | (N = 19) | (N = 125) | |
| Grade of GVHD | | | | | |
| at onset (%) | | | | | |
| 0 | 0% | 100% | 100% | 0% | |
| | (N = 0) | (N = 362) | (N = 52) | (N = 0) | |
| I | 0% | 0% | 0% | 69% | |
| | (N = 0) | (N = 0) | (N = 0) | (N = 201) | |
| Skin Stage 1 | 0% | 0% | 0% | 41% | |
| | (N = 0) | (N = 0) | (N = 0) | (N = 118) | |
| Skin Stage 2 | 0% | 0% | 0% | 29% | |
| | (N = 0) | (N = 0) | (N = 0) | (N = 83) | |
| II | 57% | 0% | 0% | 30% | |
| | (N = 96) | (N = 0) | (N = 0) | (N = 88) | |
| Isolated | 0% | 0% | 0% | 30% | |
| Skin Stage 3 | (N = 0) | (N = 0) | (N = 0) | (N = 88) | |
| Isolated Upper | 17% | 0% | 0% | 0% | |
| GI Stage 1^{‡} | (N = 29) | (N = 0) | (N = 0) | (N = 0) | |
| Lower | 40% | 0% | 0% | 0% | |
| GI Stage 1^{‡} | (N = 67) | (N = 0) | (N = 0) | (N = 0) | |
| III-IV | 43% | 0% | 0% | 1% | |
| | (N = 71) | (N = 0) | (N = 0) | (N = 1) | |
| Isolated | 0% | 0% | 0% | 1% | |
| Skin Stage 4 | (N = 0) | (N = 0) | (N = 0) | (N = 1) | |
| GI Stage 2^{‡} | 13% | 0% | 0% | 0% | |
| | (N = 22) | (N = 0) | (N = 0) | (N = 0) | |
| GI Stage 3^{‡} | 16% | 0% | 0% | 0% | |
| | (N = 27) | (N = 0) | (N = 0) | (N = 0) | |
| GI Stage 4^{‡} | 13% | 0% | 0% | 0% | |
| | (N = 22) | (N = 0) | (N = 0) | (N = 0) | |
| Day after HCT | | | | | <0.001 |
| Median | 33 | 31 | 24 | 28 | |
| (range) | (11-216) | (7-185) | (7-93) | (5-175) | |

*High risk of disease status at HCT is according to Center for International Blood and Marrow Transplant Research (CIBMTR) guidelines.
^{†}Including 29 patients with isolated upper GI GVHD and 138 with lower ± upper GI GVHD.
^{‡}With or without other GVHD target organ involvement.
^{§}Including 13 patients with isolated upper GI non-GVHD enteritis and 39 patients with lower ± upper GI non-GVHD enteritis.

TABLE 3
Causes of non-GVHD enteritis in the
University of Michigan validation set

| Non-GVHD lower GI enteritis +/− upper | | |
|---|---|---|
| GI symptoms: N = 39 | | |
| C. difficile infection | 54% | (N = 21) |
| Diarrhea w/negative biopsy | 15% | (N = 6) |
| N/V and diarrhea w/negative biopsy | 28% | (N = 11) |
| Ulcerative esophagitis and diarrhea (negative biopsies) | 3% | (N = 1) |
| Non-GVHD upper GI enteritis without | | |
| diarrhea (all biopsy negative): N = 13 | | |
| Nausea/vomiting | 54% | (N = 7) |
| Anorexia | 15% | (N = 2) |
| Chemical gastropathy | 23% | (N = 3) |
| H. pylori gastritis | 8% | (N = 1) |

Patients, i.e. human subjects, from the Regensburg/Kyushu validation set were divided into four groups as above; patient characteristics are detailed in Table 4, with causes of non-GVHD enteritis listed in Table 5.

TABLE 4
Patient characteristics of the Regensburg/Kyushu validation set.

| | | | Non- | | |
|---|---|---|---|---|---|
| | GI | No | GVHD | Skin | |
| | GVHD^{†,‡} | GVHD | Enteritis^{§} | GVHD | p- |
| Total N = 143 | N = 30 | N = 53 | N = 11 | N = 49 | value |
| Age (years) | | | | | 0.22 |
| Median | 44 | 46 | 35 | 44 | |
| (range) | (15-63) | (24-67) | (15-51) | (19-62) | |
| Disease (%) | | | | | 0.53 |
| Malignant | 97% | 92% | 91% | 98% | |
| | (N = 29) | (N = 49) | (N = 10) | (N = 48) | |
| Other | 3% | 8% | 9% | 2% | |
| | (N = 1) | (N = 4) | (N = 1) | (N = 1) | |
| Disease status at | | | | | 0.09 |
| transplant* (%) | | | | | |
| Other/low/ | 43% | 64% | 82% | 53% | |
| Intermediate risk | (N = 13) | (N = 34) | (N = 9) | (N = 26) | |
| High risk | 57% | 36% | 18% | 47% | |
| | (N = 17) | (N = 19) | (N = 2) | (N = 23) | |
| Donor type (%) | | | | | 0.96 |
| Related donor | 23% | 23% | 27% | 20% | |
| | (N = 7) | (N = 12) | (N = 3) | (N = 10) | |
| Unrelated donor | 77% | 77% | 73% | 80% | |
| | (N = 23) | (N = 41) | (N = 8) | (N = 39) | |
| Donor match (%) | | | | | 0.25 |
| Matched donor | 67% | 81% | 91% | 69% | |
| | (N = 20) | (N = 43) | (N = 10) | (N = 34) | |
| Mismatched | 33% | 19% | 9% | 31% | |
| donor | (N = 10) | (N = 10) | (N = 1) | (N = 15) | |
| Conditioning | | | | | 0.62 |
| regimen | | | | | |
| intensity (%) | | | | | |
| High intensity | 47% | 34% | 27% | 37% | |
| | (N = 14) | (N = 18) | (N = 3) | (N = 18) | |
| Moderate | 53% | 66% | 73% | 63% | |
| intensity | (N = 16) | (N = 35) | (N = 8) | (N = 31) | |
| Grade of GVHD | | | | | |
| at onset (%) | | | | | |
| 0 | 0% | 100% | 100% | 0% | |
| | (N = 0) | (N = 53) | (N = 11) | (N = 0) | |
| I | 0% | 0% | 0% | 61% | |
| | (N = 0) | (N = 0) | (N = 0) | (N = 30) | |
| Skin Stage 1 | 0% | 0% | 0% | 22% | |
| | (N = 0) | (N = 0) | (N = 0) | (N = 11) | |
| Skin Stage 2 | 0% | 0% | 0% | 39% | |
| | (N = 0) | (N = 0) | (N = 0) | (N = 19) | |
| II | 63% | 0% | 0% | 39% | |
| | (N = 19) | (N = 0) | (N = 0) | (N = 19) | |
| Isolated | 0% | 0% | 0% | 39% | |
| Skin Stage 3 | (N = 0) | (N = 0) | (N = 0) | (N = 19) | |
| Isolated Upper | 20% | 0% | 0% | 0% | |
| GI Stage 1^{‡} | (N = 6) | (N = 0) | (N = 0) | (N = 0) | |
| Lower | 43% | 0% | 0% | 0% | |
| GI Stage 1^{‡} | (N = 13) | (N = 0) | (N = 0) | (N = 0) | |
| III-IV | 37% | 0% | 0% | 0% | |
| | (N = 11) | (N = 0) | (N = 0) | (N = 0) | |
| Isolated | 0% | 0% | 0% | 0% | |
| Skin Stage 4 | (N = 0) | (N = 0) | (N = 0) | (N = 0) | |
| GI Stage 2^{‡} | 17% | 0% | 0% | 0% | |
| | (N = 5) | (N = 0) | (N = 0) | (N = 0) | |
| GI Stage 3^{‡} | 7% | 0% | 0% | 0% | |
| | (N = 2) | (N = 0) | (N = 0) | (N = 0) | |
| GI Stage 4^{‡} | 13% | 0% | 0% | 0% | |
| | (N = 4) | (N = 0) | (N = 0) | (N = 0) | |
| Day after HCT | | | | | 0.7 |
| Median | 19 | 26 | 28 | 20 | |
| (range) | (8-182) | (14-86) | (14-51) | (11-485) | |

*High risk of disease status at HCT is according to Center for International Blood and Marrow Transplant Research (CIBMTR) guidelines.
^{†}Including 6 patients with isolated upper GI GVHD and 24 with lower ± upper GI GVHD.
^{‡}With or without other GVHD target organ involvement.
^{§}Including 8 patients with isolated upper GI non-GVHD enteritis and 3 patients with lower ± upper GI non-GVHD enteritis.

TABLE 5
Causes of non-GVHD enteritis in the
Regensburg/Kyushu validation set

| | |
|---|---|
| Non-GVHD lower GI enteritis +/− upper | |
| GI symptoms: N = 3 | |
| C. difficile infection | 33% (N = 1) |
| Diarrhea; biopsy negative | 33% (N = 1) |
| Diarrhea; no biopsy, spontaneously resolved | 33% (N = 1) |
| Non-GVHD upper GI enteritis without diarrhea: N = 8 | |
| Nausea/vomiting; biopsy negative | 75% (N = 6) |
| Nausea/vomiting; no biopsy, spontaneously resolved | 12% (N = 1) |
| CMV gastritis | 13% (N = 1) |

### Histopathology

GI biopsies were obtained and prepared per institutional guidelines. GVHD was histologically confirmed by duodenal/colonic biopsy in 183 of 197 GI GVHD patients and by skin biopsy in an additional five subjects with both rash and GI symptoms. Skin GVHD was confirmed by biopsy in 272 of 341 subjects with rashes and by biopsy of another target organ later affected by GVHD in an additional eight subjects. 162 subjects of 197 subjects with GVHD had diarrhea. 140 of these 162 subjects had biopsies (duodenal=87, colonic=53) available for formal grading as described by Lerner et al. (Transplant Proc. 6: 367-71, 1974). If both duodenal and colonic biopsies were available, colonic biopsies were graded only if duodenal biopsies were negative. Values for unavailable biopsies were not imputed. Paneth cells were counted in four high-power fields (HPFs) in the area of each biopsy showing the largest number of Paneth cells per specimen using an Olympus BX43 microscope; a HPF was defined as a 40× objective (0.345 mm²). The counts from the four fields were then averaged to give the number of Paneth cells per HPF.

### ELISA Assays

REG3α ELISA kits were purchased from MBL International (Woburn, Mass.; Ab-Match Assembly Human PAP1 kit and Ab-Match Universal kit), and measurements were performed according to the manufacturer's protocol. Samples (diluted 1:10) and standards were run in duplicate. Absorbance was measured with a SpectraMax M2 (Molecular Devices, Sunnyvale, Calif.), and results were calculated with SoftMax Pro v5.4 (Molecular Devices). Elafin, IL2Rα, HGF, TNFR1, and IL-8 ELISAs were performed in duplicate as previously reported (Paczesny et al., Sci. Transl. Med. 2: 50-7, 2010; Paczesny et al., Blood 113: 273-8, 2009). Measurements of samples from 66 subjects (6.5% of the total population) were repeated in a second ELISA at random intervals and were comparable; correlation coefficient r=0.82, p<0.0001. Details of the assay parameters are provided in Table 6.

TABLE 6
ELISA assay parameters

| | | | | | ULOD | LLOD |
|---|---|---|---|---|---|---|
| | STD Curve | | Dilution | CV | (optical | (optical |
| | Range | | Factor | %* | density) | density) |
| REG3α | 100-1.6 | ng/ml | 1/10 | 5.91 | 1.80 ± 0.13 | 0.04 ± 0.08 |
| IL-2Rα | 2000-31.2 | pg/ml | Un- | 2.59 | 1.11 ± 0.29 | 0.03 ± 0.02 |
| | | | diluted | | | |
| TNFR1 | 800-12.5 | pg/ml | 1/25 | 4.23 | 1.64 ± 0.36 | 0.05 ± 0.03 |
| Elafin | 2000-31.2 | pg/ml | 1/20 | 6.46 | 2.26 ± 0.63 | 0.16 ± 0.05 |
| HGF | 4000-62.5 | pg/ml | ½ | 2.35 | 1.96 ± 0.60 | 0.07 ± 0.11 |
| IL-8 | 200-3.1 | pg/ml | ⅙ | 7.13 | 1.86 ± 0.76 | 0.03 ± 0.04 |

*CV calculated on 3^{rd} highest standard concentration; CV = (standard deviation/mean)*100.

### Statistical Analysis

The statistical methods used for the IPAS were previously described (Faca et al., J. Proteome Res. 5: 2009-18, 2006; Faca et al., J. Proteome Res. 6: 3558-65, 2007; Paczesny et al., Sci. Transl. Med. 2: 50-7, 2010). REG3α and albumin concentrations from individual samples in the discovery and validation sets (described in more detail in the Examples herein below) were compared using two-sample t-tests applied to log-transformed concentrations. Differences in characteristics between subject groups were assessed with a Kruskal-Wallis test for continuous values and chi-squared tests of association for categorical values. Receiver operating characteristic (ROC) areas under the curves (AUC) were estimated nonparametrically. Non-relapse mortality (NRM) and relapse mortality were modeled with cumulative incidence regression methods as described by Fine et al. (J. Am. Stat. Assoc. 94: 496-509, 1999). 1-year overall survival (OS) was modeled with Cox regression methods and probability of response was modeled with logistic regression.

### Example 2

### Discovery Study

The objective of this discovery study was to identify candidate biomarkers for GVHD using a proteomics approach to identify candidate biomarkers in a discovery set of pooled plasma samples taken at similar times after HCT from ten subjects with biopsy-proven GI GVHD and ten subjects without GVHD (see Table 1 above).

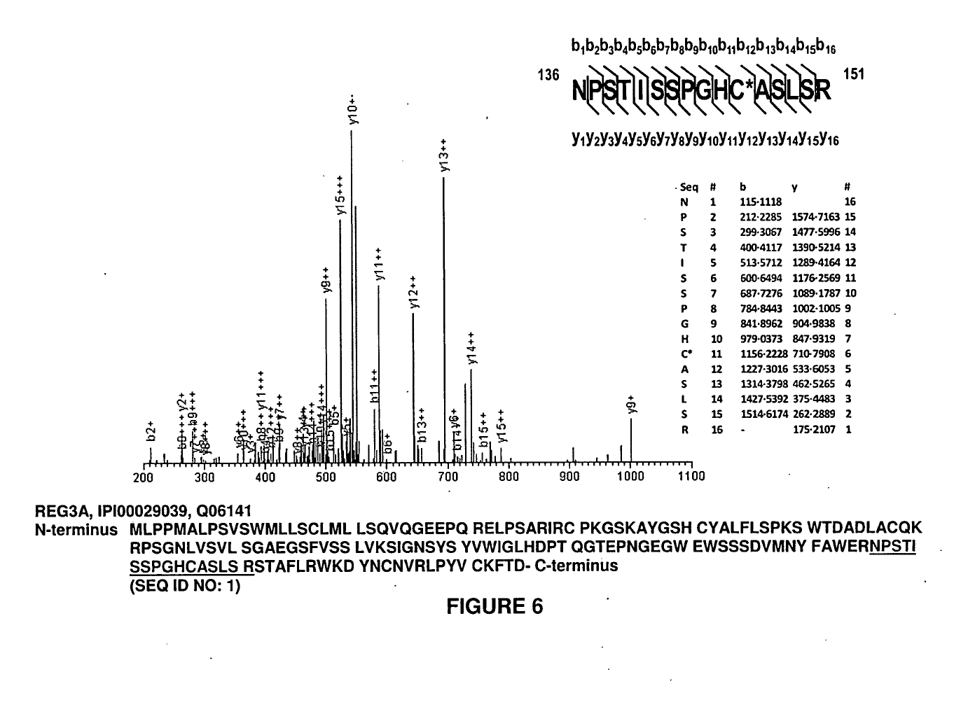

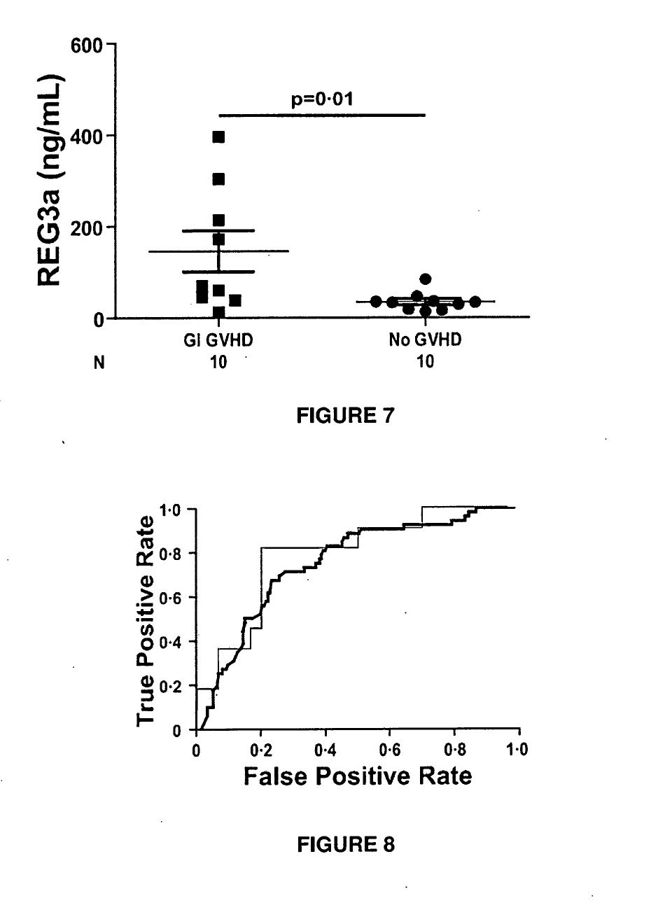

562 proteins were identified and quantified of which 74 were increased at least two-fold in subjects with GVHD (Table 7). Five proteins (carboxypeptidase N catalytic chain precursor, pancreatic secretory trypsin inhibitor precursor, palladin, lithostathine 1-alpha precursor, and regenerating Islet-derived 3-alpha (REG3α)) were preferentially expressed in the GI tract. Commercially available antibodies suitable for quantification of plasma concentrations by ELISA were available for only one of these five proteins, REG3α (Table 7): The MS characteristics of the identified REG3α peptides are shown in FIG. 6 and Table 8. The plasma concentrations of REG3α in the individual plasma samples in the discovery set were four times greater in subjects with GI GVHD than in asymptomatic controls (FIG. 7, p=0•01).

TABLE 7
GI GVHD candidate biomarkers identified by IPAS

| | | | RATIO | | Preferential GI | Suitable |
|---|---|---|---|---|---|---|
| IPI* | Gene Name | Gene Description | (mean) | #Events | expression^{†} | antibodies ^{‡} |
| IPI00032214 | BRD1 | Bromodomain-containing protein 1. | 35.5 | 1 | No | No |
| IPI00012549 | OCDHGA11 | Isoform 2 of protocadherin gamma a11 | 34.0 | 1 | No | No |
| | | precursor. | | | | |
| IPI00738813 | OXR1 | Oxidation Resistance Protein 1 | 25.1 | 1 | No | No |
| IPI00456604 | FAM19A1 | Family with sequence similarity 19, | 12.0 | 1 | No | No |
| | | member A1 precursor | | | | |
| IPI00060310 | PLD4 | Phospholipase d4. | 11.8 | 1 | No | No |
| IPI00100668 | GBA2 | Isoform 1 of non-lysosomal | 11.7 | 1 | No | No |
| | | glucosylceramidease. | | | | |
| IPI00010295 | CPN1 | Carboxypeptidase N catalytic chain | 8.9 | 2 | Yes | No |
| | | precursor. | | | | |
| IPI00010779 | TPM4 | Isoform 1 of tropomyosin alpha-4 chain. | 8.4 | 1 | No | Yes |
| IPI00410143 | CENPM | Isoform 2 of centromere protein m. | 7.7 | 2 | No | No |
| IPI00305698 | GGCX | Vitamin k-dependent gamma- | 7.6 | 1 | No | No |
| | | carboxylase. | | | | |
| IPI00012011 | CFL1 | Cofilin, non-muscle isoform | 7.5 | 9 | No | No |
| IPI00059279 | EXOC4 | Exocyst complex component 4 | 7.5 | 2 | No | No |
| IPI00020687 | SPINK1 | Pancreatic secretory trypsin inhibitor | 7.4 | 4 | Yes | No |
| | | precursor | | | | |
| IPI00022417 | LRG1 | Leucine-rich alpha-2-glycoprotein | 7.2 | 1 | No | Yes |
| | | precursor. | | | | |
| IPI00005822 | CDC23 | Cell division cycle protein 23. | 7.0 | 2 | No | No |
| IPI00009822 | SRP54 | Signal recognition particle 54 kDa | 7.0 | 4 | No | No |
| | | protein | | | | |
| IPI00008274 | CAP1 | Adenylyl cyclase-associated protein 1 | 6.1 | 2 | No | No |
| IPI00009143 | ADAMTS5 | ADAM metallopeptidase with | 5.1 | 1 | No | Yes |
| | | thrombospondin type 1 motif, 5 | | | | |
| | | precursor | | | | |
| IPI00292950 | SERPIND1 | Heparin cofactor II precursor | 4.8 | 7 | No | Yes |
| IPI00036578 | ADAMTS12 | ADAM metallopeptidase with | 4.4 | 1 | No | No |
| | | thrombospondin type 1 motif, 12 | | | | |
| | | preproprotein | | | | |
| IPI00299155 | PSMA4 | Proteasome subunit alpha type 4. | 4.3 | 2 | No | No |
| IPI00216691 | PFN1 | Profilin-1 | 4.2 | 3 | No | No |
| IPI00290420 | HPGD | 15-hydroxy prostaglandin dehydrogenase. | 4.1 | 1 | No | No |
| IPI00304922 | LSMD1 | LSM domain containing 1 | 4.0 | 1 | No | No |
| IPI00477868 | LAMA5 | Laminin, alpha 5 | 3.8 | 1 | No | No |
| IPI00414467 | COLEC12 | Nurse cell scavenger receptor 2 | 3.8 | 1 | No | No |
| IPI00011155 | ASGR2 | Splice Isoform 1 of Asialoglycoprotein | 3.7 | 1 | No | No |
| | | receptor 2 | | | | |
| IPI00294615 | FBLN5 | Fibulin-5 precursor | 3.5 | 1 | No | No |
| IPI00376787 | EZH2 | Enhancer of zeste 2 isoform a. | 3.3 | 1 | No | No |
| IPI00293276 | MIF | Macrophase migration inhibitory factor | 3.3 | 2 | No | Yes |
| IPI00006971 | CD248 | Tumor endothelial marker 1 | 3.3 | 2 | No | No |
| IPI00184019 | PILRA | Paired immunoglobin-like receptor alpha | 3.2 | 1 | No | No |
| IPI00019372 | PRG1 | Secretory granule proteoglycan core | 3.2 | 1 | No | No |
| | | protein precursor | | | | |
| IPI00018136 | VCAM1 | Splice Isoform 1 of Vascular cell | 3.1 | 11 | No | No |
| | | adhesion protein 1 precursor | | | | |
| IPI00022585 | AKAP1 | Isoform 1 of a kinase anchor protein 1, | 3.0 | 1 | No | No |
| | | mitochondrial precursor. | | | | |
| IPI00239077 | HINT1 | Histidine triad nucleotide-binding | 2.9 | 1 | No | No |
| | | protein 1 | | | | |
| IPI00166197 | PALLD | Palladin | 2.9 | 1 | Yes | No |
| IPI00009027 | REG1A | Lithostathine 1 alpha precursor | 2.9 | 3 | Yes | No |
| IPI00298547 | PARK7 | Protein DJ-1 | 2.9 | 2 | No | No |
| IPI00218288 | SEC24D | Sec24-related protein D | 2.8 | 2 | No | No |
| IPI00010341 | PRG2 | Eosinophil granule major basic protein | 2.8 | 5 | No | No |
| | | precursor | | | | |
| IPI00299977 | PHPT1 | 14 kDa phosphohistidine phosphatase | 2.8 | 1 | No | No |
| IPI00004656 | B2M | Beta-2-microglobulin precursor | 2.8 | 48 | No | Yes |
| IPI00030154 | PSME1 | Proteasome activator complex subunit 1 | 2.6 | 3 | No | No |
| IPI00326257 | AP1B1 | Isoform a of ap-1 complex subunit beta-1. | 2.6 | 1 | No | No |
| IPI00291866 | SERPING1 | Plasma protease C1 inhibitor precursor | 2.6 | 60 | No | No |
| IPI00001458 | KNTC1 | Kinetochore-associated protein 1. | 2.6 | 1 | No | No |
| IPI00002436 | CNOT4 | Isoform 5 of ccr4-not transcription | 2.6 | 1 | No | No |
| | | complex subunit 4. | | | | |
| IPI00027848 | MRC1 | Macrophage mannose receptor 1 precursor. | 2.6 | 1 | No | No |
| IPI00006717 | CCL16 | Small inducible cytokine A16 precursor | 2.6 | 1 | No | Yes |
| IPI00022429 | ORM1 | Alpha-1-acid glycoprotein 1 precursor | 2.5 | 192 | No | Yes |
| IPI00296713 | GRN | Splice Isoform 1 of Granulins precursor | 2.5 | 1 | No | Yes |
| IPI00022200 | COL6A3 | AlphA 3 type VI collagen isoform 1 | 2.4 | 1 | No | No |
| | | precursor | | | | |
| IPI00419585 | PPIA | Peptidyl-prolyl cis-trans isomerase A | 2.4 | 2 | No | No |
| IPI00022284 | PRNP | Major prion protein precursor | 2.4 | 1 | No | Yes |
| IPI00032292 | TIMP1 | Metalloproteinase inhibitor 1 precursor | 2.3 | 9 | No | Yes |
| IPI00022418 | FN1 | Splice Isoform 1 of Fibronectin precursor | 2.2 | 224 | No | Yes |
| IPI00414283 | FN1 | Fibronectin 1 isoform 1 preproprotein. | 2.2 | 36 | No | Yes |
| IPI00029039 | REG3A | Regenerating islet-derived protein 3 | 2.2 | 4 | Yes | Yes |
| | | alpha precursor | | | | |
| IPI00005769 | FANCG | Fanconi anemia group g protein. | 2.2 | 1 | No | No |
| IPI00164104 | DLEC1 | Isoform 1 of deleted in lung and | 2.2 | 1 | No | No |
| | | esophageal cancer protein 1 | | | | |
| IPI00008148 | GFRA1 | Isoform 1 of gdnf family receptor | 2.1 | 1 | No | No |
| | | alpha-1 precursor. | | | | |
| IPI00103636 | WFDC2 | Splice Isoform 2 of WAP four disulfide | 2.1 | 2 | No | No |
| | | core domain protein 2 precursor | | | | |
| IPI00376005 | EIF5A | Isoform 2 of eukaryotic translation | 2.1 | 1 | No | No |
| | | initiation factor 5a-1. | | | | |
| IPI00026941 | PRSS23 | Serine protease 23 precursor | 2.1 | 2 | No | No |
| IPI00025155 | FSTL3 | Follistatin-related protein 3 precursor. | 2.1 | 1 | No | Yes |
| IPI00013831 | CD48 | B-lymphocyte activation marker BLAST-1 | 2.1 | 1 | No | No |
| | | precursor | | | | |
| IPI00295339 | SELP | P-selectin precursor | 2.1 | 1 | No | Yes |
| IPI00760855 | TMEM110 | Transmembrane protein 110 | 2.0 | 3 | No | No |
| IPI00030144 | PPIAL4 | Preptidyl-prolyl cis-trans isomerase. | 2.0 | 1 | No | No |
| IPI00479186 | PKM2 | Pyruvate kinase 3 isoform 1 variant | 2.0 | 2 | No | No |
| IPI00015029 | PTGES3 | Telomerase-binding protein p23 | 2.0 | 3 | No | No |
| IPI00029623 | PSMA6 | Proteasome subunit alpha type 6 | 2.0 | 2 | No | No |
| IPI00219018 | GAPDH | Glyceraldehyde-3-phophate | 2.0 | 3 | No | No |
| | | dehydrogenase, liver | | | | |

*IPI: International Protein Index
^{†}Preferential GI expression: proteins expressed in GI tract but not in skin, bone marrow or lymphoid tissue, as referred by gene ontology, human protein atlas and literature search
^{‡} Suitable antibodies: Established antibody pairs for ELISA screening.

TABLE 8
REG3A proteomic analysis

| Peptide Level | | | | | | | | | | | Peptide | |
|---|---|---|---|---|---|---|---|---|---|---|---|---|
| IPI | Z | Time | PreMass | CalMass | dMppm | Expect | q3L | q3H | NRatio | Prob | Sequence | dbHit |
| IPI00029039 | 2 | 1626.3 | 1686.83 | 1686.81 | 11 | 0.098 | 0 | 329190 | 9999 | 0.97 | NPSTISSPGHC[C | 1 |
| | | | | | | | | | | | ysH]ASLSR | |
| IPI00029039 | 3 | 1729.8 | 1686.83 | 1686.81 | 10 | 0.054 | 0 | 108078 | 9999 | 1 | NPSTISSPGHC[C | 1 |
| | | | | | | | | | | | ysH]ASLSR | |
| IPI00029039 | 2 | 1730.1 | 1686.83 | 1686.81 | 14 | 0.029 | 0 | 67488 | 9999 | 1 | NPSTISSPGHC[C | 1 |
| | | | | | | | | | | | ysH]ASLSR | |
| IPI00029039 | 3 | 1641.6 | 1686.83 | 1686.81 | 13 | 0.28 | 0 | 5991 | 9999 | 0.97 | NPSTISSPGHC[C | 1 |
| | | | | | | | | | | | ysH]ASLSR | |
| IPI00029039 | 2 | 1609.4 | 1687.84 | 1686.81 | 15 | 0.085 | 0 | 25367 | 9999 | 0.86 | NPSTISSPGHC[C | 1 |
| | | | | | | | | | | | ysH]ASLSR | |
| IPI00029039 | 3 | 1885.1 | 1687.83 | 1686.81 | 8 | 0.64 | 3852 | 6344 | 1.6 | 0.91 | NPSTISSPGHC[C | 1 |
| | | | | | | | | | | | ysH]ASLSR | |
| IPI00029039 | 3 | 1500.9 | 1686.83 | 1686.81 | 14 | 0.1 | 11429 | 23797 | 2.0 | 0.99 | NPSTISSPGHC[C | 1 |
| | | | | | | | | | | | ysH]ASLSR | |
| IPI00029039 | 3 | 1597.4 | 1686.83 | 1686.81 | 14 | 0.39 | 0 | 5007 | 9999 | 0.99 | NPSTISSPGHC[C | 1 |
| | | | | | | | | | | | ysH]ASLSR | |
| IPI00029039 | 3 | 1836.6 | 1686.83 | 1686.81 | 11 | 2.2 | 740 | 1279 | 1.7 | 0.81 | NPSTISSPGHC[C | 1 |
| | | | | | | | | | | | ysH]ASLSR | |
| IPI00029039 | 2 | 2462 | 1310.6 | 1310.59 | 8 | 0.017 | 0 | 341159 | 9999 | 1 | SWTDADLAC[C | 1 |
| | | | | | | | | | | | ysH]QK | |
| IPI00029039 | 2 | 2565.4 | 1310.6 | 11310.59 | 12 | 0.012 | 23969 | 103841 | 4.0 | 1 | SWTDADLAC[C | 1 |
| | | | | | | | | | | | ysH]QK | |
| Big Quant Level | | | | | | | | | | | | |
| IPI | Chr | Length | MW | GMean | Events | StDev | TTest | Pvalue | | | | |
| IPI00029039 | 2 | 175 | 19395 | 2.2 | 4 | 0.43 | 3.6 | 0.0343 | | | | |

IPI: International Protein Index; Z: charge, PreMass: Precursor Mass; CalMass: Calculated Mass; dMppm: fractional delta mass in part per million; Expect: Expected value; q3L: quantified Light value; q3H quantified Heavy value; Nratio: Normalized ratio; Prob: Peptide Probability; dbHits: number of indistinguishable hits in IPI database; GMean: geometric mean.

### Example 3

### Validation Study

The objective of this validation study was to determine if REG3α, identified as a biomarker in the discovery study, is a valid biomarker of GI GVHD. Plasma REG3α concentrations in samples from a validation set of 871 allogeneic HCT recipients from the University of Michigan (see Table 2 above) were measured. Older transplant recipients, an underlying diagnosis of malignant disease, graft sources from unrelated and HLA-mismatched donors were over-represented in the groups with GVHD. The median day of sample acquisition for subjects with non-GVHD enteritis was closer to the day of transplant than for all other groups.

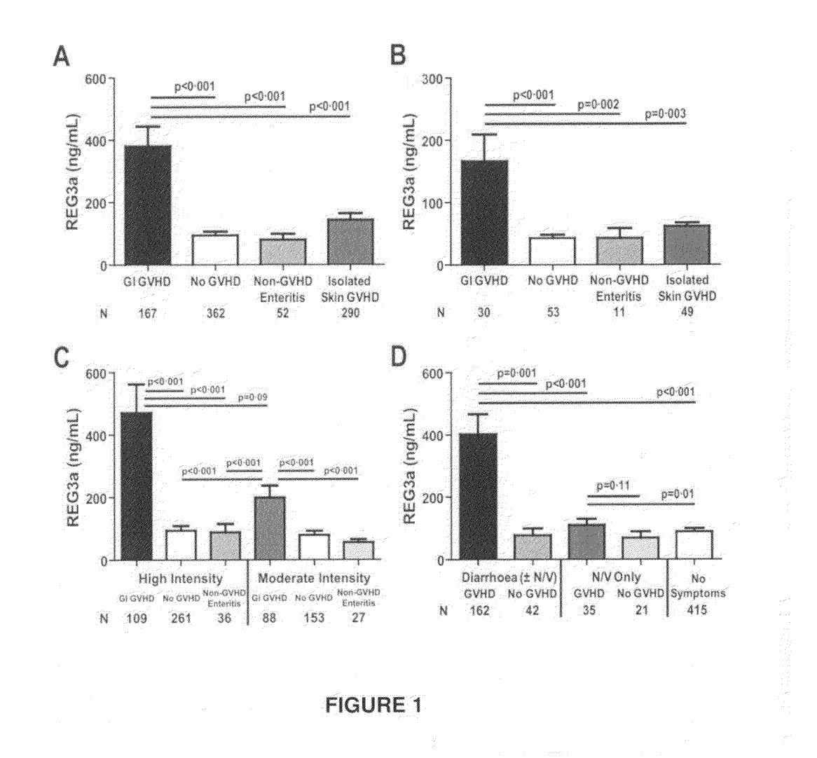

Plasma REG3α concentrations were three times greater in subjects at the onset of GI GVHD than in all other subjects, including those with non-GVHD enteritis (FIG. 1A). Serum REG3α concentrations were also greater in GI GVHD in an independent validation set of 143 HCT subjects from Regensburg, Germany, and Kyushu, Japan, although the absolute values were lower (FIG. 1B). This difference may be due to a center effect that depends on several factors, including variations in transplant conditioning regimens and supportive care. Subjects receiving high intensity conditioning regimens had REG3α concentrations that were twice as high as those receiving moderate intensity conditioning, but this difference did not reach statistical significance (FIG. 1C). In addition, all subjects in Regensburg and Kyushu received oral antibiotics as GVHD prophylaxis, whereas Michigan subjects did not. Without being bound by theory, increased GI flora could account for greater REG3α secretion.

REG3α concentrations were next analyzed according to diagnosis and type of GI symptom. In subjects with diarrhea caused by GVHD, REG3α concentrations at the onset of GVHD were five times greater than in subjects with diarrhea from other causes (FIG. 1D). In subjects without diarrhea, REG3α concentrations were 25% greater when attributable to GVHD compared to other causes, a difference that was not statistically significant.

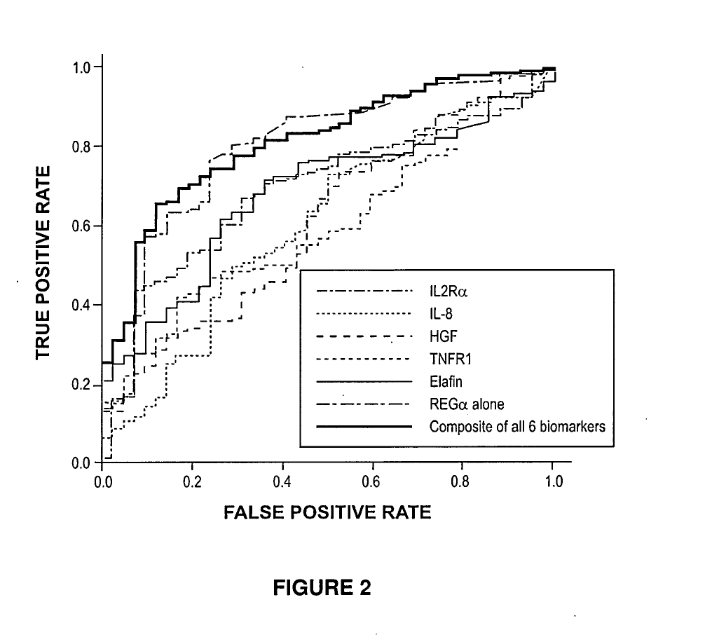

Concentrations of four previously reported diagnostic markers of systemic acute GVHD (IL2Rα, TNFR1, IL-8, and HGF) and of elafin, a biomarker for GVHD of the skin, in all subjects with diarrhea (FIG. 1C, N=204) were measured. ROC curves for these biomarkers distinguished GVHD from non-GVHD with an area under the curve (AUC) of 0•80 for REG3α alone and an AUC of 0•81 for a composite panel of all six biomarkers (FIG. 2). In this analysis, 52% of subjects with lower GI GVHD also had skin involvement at onset and, thus, the AUC for elafin, which is specific for GVHD of the skin, was greater than expected (Table 9). ROC curves of REG3α concentrations in subjects with diarrhea had similar AUCs in both validation sets (FIG. 8). REG3α was therefore the best single diagnostic biomarker at the onset of symptoms of lower GI GVHD, and additional biomarkers provided no further increased sensitivity or specificity.

TABLE 9
GVHD target organ involvement at onset of GVHD.

| | |
|---|---|
| Isolated skin GVHD | 339 |
| Isolated GI GVHD* | 118 |
| HI GVHD plus skin GVHD† | 79 |

*Including 9 subjects with liver GVHD
†Including 13 subjects with liver GVHD

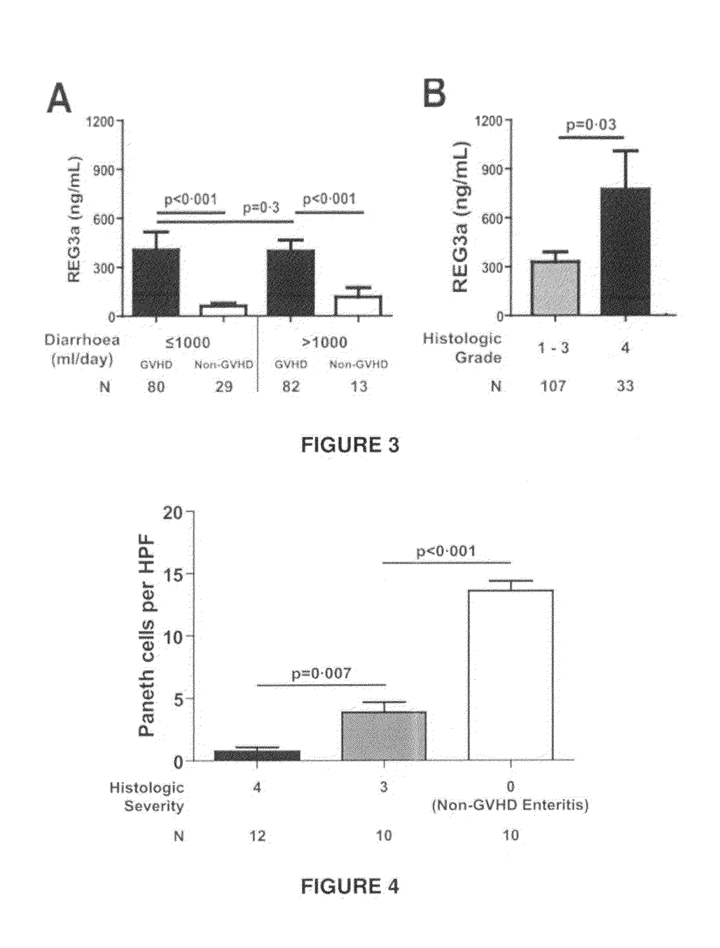

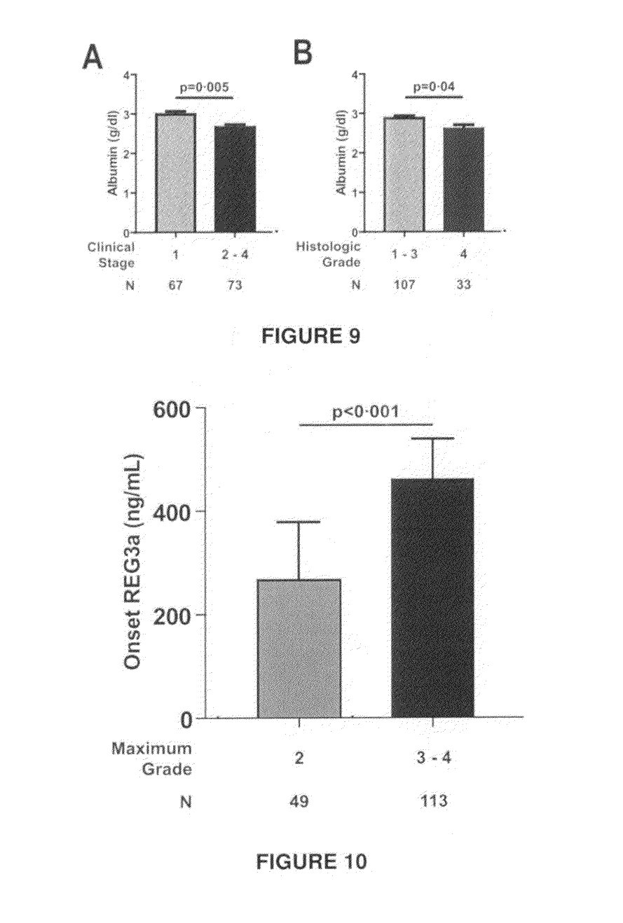

When subjects were categorized by volume of diarrhea, REG3α concentrations at the onset of symptoms continued to distinguish between GVHD and non-GVHD etiologies (FIG. 3A, p<0•001), but did not correlate with the clinical stage of GVHD. 23 of 26 subjects with clinical stage 4 GI GVHD at onset received full intensity conditioning, and these subjects showed a trend toward greater REG3α concentrations than those with stages 1-3 GI GVHD (p=0•07; data not shown). Plasma REG3α concentrations at the onset of GVHD were significantly greater in subjects whose GI biopsies showed evidence of severe GVHD with mucosal denudation (histologic grade 4) compared to less severe GVHD (FIG. 3B; p=0•03). The number of Paneth cells present in biopsies decreased as the histologic grade of GVHD increased (FIG. 4). Hypoalbuminemia is associated with the protein-losing enteropathy in GI GVHD (Weisdorf et al., Gastroenterology 85: 1076-81, 1983); thus, serum albumin level was analyzed as a potential marker for loss of intravascular proteins into the intestinal lumen. Albumin levels at the onset of GI GVHD also correlated with both the clinical GI GVHD severity (FIG. 9A) and, histopathologic severity (FIG. 9B).

### Example 4

### Prognostic Value of REG3α Concentrations in Subjects with Lower GI GVHD

The clinical utility of any biomarker is greatly enhanced when it provides prognostic information regarding the future status of a disease and/or subject, e.g. the likelihood of response to treatment. Therefore, the prognostic value of REG3α plasma levels in 162 subjects taken at the time of diagnosis of lower GI GVHD was evaluated.

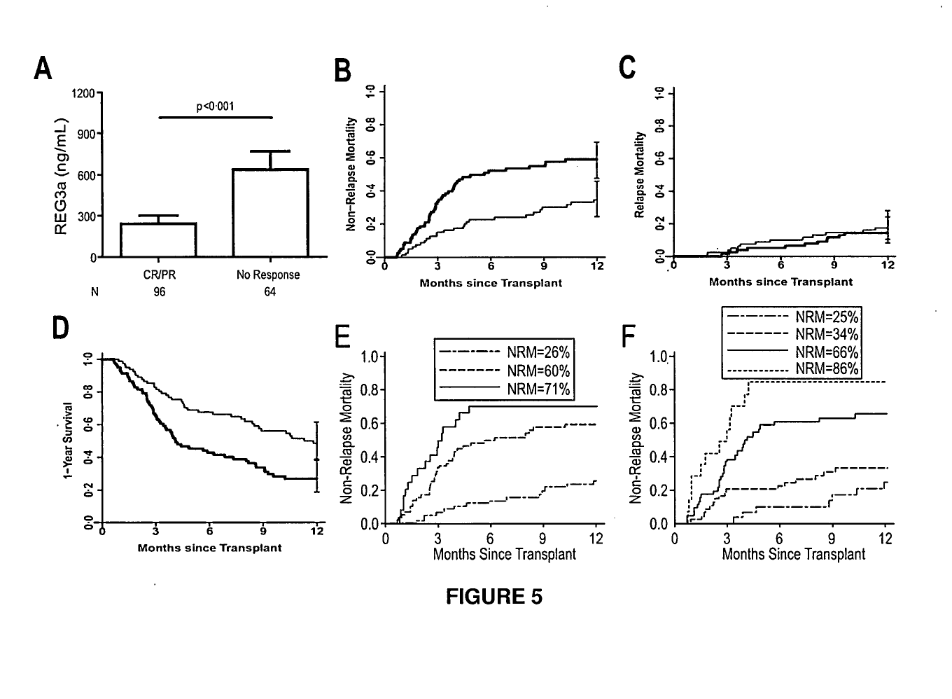

REG3α concentrations were three-fold greater at the time of GVHD diagnosis in subjects who had no response to therapy at four weeks than in subjects who experienced a complete or partial response (FIG. 5A; p<0•001). Subjects responding to therapy still exhibited REG3α concentrations more than twice that of non-GVHD controls. REG3α concentrations at diagnosis also correlated with eventual maximal clinical stage of GI GVHD (FIG. 10).

Because maximal GVHD grade correlates with NRM (Weisdorf et al., Blood 75: 1024-30, 1990), it was hypothesized that REG3α concentration at GVHD diagnosis would also correlate with NRM. To test this hypothesis, 162 subjects were divided into two equal groups based upon median REG3α concentration: high (>151 ng/ml, n=81) and low (≦151 ng/ml, N=81). NRM was twice as high in subjects with high REG3α concentrations, and this difference remained significant after adjusting for known risk factors of donor type, degree of HLA match, conditioning intensity, age, and baseline disease severity (59% [95% CI 48-69%] vs. 34% [95% CI 24-46%], p<0•001, FIG. 5B). The incidence of relapse mortality was comparable for both groups (14% [95% CI 8-24] vs. 17% [95% CI 8-24], p=0•5; FIG. 5C); subjects with high REG3α concentrations at the time of GVHD diagnosis experienced significantly inferior one-year OS (27% [95% CI 19-39%] vs. 48% [95% CI 38-61%], p=0•001; FIG. 5D). Causes of one-year mortality for these subjects are listed in Table 10.

TABLE 10
Causes of 1-year mortality in lower GI GVHD subjects (N = 97).

| | | |
|---|---|---|
| Non-relapse mortality | 79% | (N = 77) |
| Acute GVHD | 65% | (N = 50) |
| Infection/sepsis | 12% | (N = 9) |
| Chronic GVHD | 12% | (N = 9) |
| Graft failure | 3% | (N = 2) |
| Multiple organ failure | 1% | (N = 1) |
| SOS | 1% | (N = 1) |
| Intracranial hemorrhage | 1% | (N = 1) |
| Unknown | 5% | (N = 4) |
| Relapse mortality | 21% | (N = 20) |

Of the 162 subjects with diarrhea at the onset of GVHD, all four data points, (1) clinical stage, (2) histologic grade, (3) REG3α concentration and (4) serum albumin level were evaluated in 140 subjects. As shown in Table 11, the plasma concentration of REG3α, the clinical severity of GVHD, the histologic severity, and serum albumin level at GVHD diagnosis independently predicted lack of response to GVHD therapy four weeks following treatment after adjustment for the aforementioned risk factors (odds ratios: 4•8, 3•9, 18•9, and 2•5, respectively). When lack of response to therapy and NRM were modeled simultaneously on all four parameters, all but albumin remained statistically significant. When only advanced clinical stage and severe histologic grade were considered, NRM was 71% (FIG. 5E), but the inclusion of high REG3α concentration produced a significantly greater NRM of 86% for subjects with all three risk factors (FIG. 5F, p<0•001).

TABLE 11
REG3α concentrations and characteristics at onset of GVHD
diarrhea predict 4-week response to GVHD therapy and 1-year NRM.

| | Independent | | Simultaneous | |
|---|---|---|---|---|
| No response to treatment | Odds | p- | Odds | p- |
| (at 4 weeks) | Ratio | value* | Ratio | value* |
| REG3 (high vs. low) | 4.8 | <0.001 | 5.7 | 0.001 |
| GVHD GI onset state (2-4 vs. 1) | 3.9 | 0.001 | 3.0 | 0.027 |
| Histologic grade (4 vs. 1-3) | 18.9 | <0.001 | 16.7 | <0.001 |
| Albumin (low vs. high) | 2.5 | 0.02 | 1.4 | 0.5 |

| | Independent | | Simultaneous | |
|---|---|---|---|---|
| | Hazard | p- | Hazard | p- |
| 1-Year NRM | Ratio | value* | Ratio | value* |
| REG3 (high vs. low) | 2.2 | 0.003 | 2.4 | 0.002 |
| GVHD GI onset state (2-4 vs. 1) | 3.0 | <0.001 | 3.1 | <0.001 |
| Histologic grade (4 vs. 1-3) | 3.6 | <0.001 | 2.9 | <0.001 |
| Albumin (low vs. high) | 2.3 | 0.004 | 1.6 | 0.2 |

*Adjusted for age, donor type, HLA match, conditioning intensity and disease status at transplant.

TABLE 12
Positive (PPV) and negative (NPV) predictive values for GI GVHD
of plasma REG3α concentrations at the onset of diarrhea.

| Cutoff | PPV | NPV |
|---|---|---|
| 151 ng/ml (50%-ile*) | 95% | 32% |
| 100 ng/ml (42%-ile*) | 95% | 35% |
| 57 ng/ml (25%-ile*) | 92% | 44% |
| 28 ng/ml (10%-ile*) | 84% | 50% |

*%-ile of REG3α concentration in patients with lower GI GVHD at onset.

### Example 5

### Predictive Ability of REG3α in Preemptive Treatment of GVHD

To determine whether biomarkers can predict the occurrence of clinically significant Grade II-IV GVHD before the onset of clinical symptoms, samples that were obtained prospectively from subjects on day +7 and day +14 after bone marrow transfer (BMT) were tested. These two particular days (days +7 and +14) were chosen for testing because the median day of onset of GVHD has been determined to be about day +23. Thus, to be useful in predicting GVHD, it is contemplated that a biomarker should accurately predict the occurrence of GVHD several days or more before the onset of symptoms.

When subjects were divided into a training set and a validation set, the measuring three markers of IL2Rα, REG3α and Elafin on days +7 and +14 gave the best sensitivity and specificity (68% and 50%, respectively). This biomarker panel is therefore sufficiently sensitive and specific to correctly predict the future occurrence of GVHD in the majority of subjects at risk and can be used to guide preemptive therapy for GVHD.

### Example 6

### Measuring REG3α to Determine Responsivity to Treatment for GVHD

To determine whether. REG3α level in subject can demonstrate responsivity to treatment for GVHD; blood samples are obtained from subjects at the time of diagnosis of GVHD and then at various intervals after the onset of treatment for GVHD. For example, blood samples are taken from a subject undergoing GVHD treatment at days 7, 14, 21, 28, 35, 42, and then weekly or monthly thereafter.

When subjects are responsive to treatment with GVHD, either first line therapy or second line therapy, there is a significant reduction in REG3α level in the blood sample of the subject. This biomarker is therefore sufficiently sensitive and specific to correctly demonstrate responsiveness to therapy in the treatment of GVHD.

### Example 7

### Plasma Concentration of ST2 at Initiation of GVHD Therapy Predicts Day 28 Response and Day 180 Survival Post-Treatment

Acute GVHD is the primary limitation of HCT. Current diagnostic tests do not predict a patient's response to therapy, particularly at GVHD onset, when risk-stratification is most beneficial. It would be valuable for clinicians to have a marker to predict non-response because it is related to mortality. Thus, a major challenge for clinicians is to identify which patients will respond to current GVHD treatment and to design more efficient treatment regimens. The ability to identify patients who will not respond to traditional treatment and who are at particularly high risk for morbidity and mortality could permit tailored treatment plans, such as additional immunosuppressive treatments for high-risk patients that may be more effective if introduced early. Equally important is the identification of low-risk patients who will respond well to treatment. These patients may tolerate a more rapid tapering of steroid regimens to reduce long-term toxicity, infections, and a loss of the graft versus leukemia effect. Follow-up marker monitoring in high-risk patients could also help decide whether to taper the treatment.

To identify a biomarker or panel of biomarkers that could predict therapy responsiveness, an intact proteomic analysis system (IPAS) approach (Paczesny et al., Sci. Transl. Med. 2:13ra2, 2010) was used to compare pooled plasma taken at D16±5 post-therapy from 10 responders (R) and 10 non-responders (NR). Ten candidate biomarkers with an NR/R ratio of >1.5 in the IPAS were measurable by ELISA. Biomarker concentrations were measured in the 20 individual plasma aliquots. Five biomarkers (ST2, IL1sRII, MIF, LYVE, and Lipocalin) were significantly increased in NR vs. R, with an area under the receiver operator characteristic curve of 0.85. These biomarker levels were then measured at therapy initiation (DO), with 6 previously validated diagnostic biomarkers of GVHD (IL2Rα, TNFR1, HGF, IL8, Elafin, a skin-specific marker, and Reg3α) in plasma samples from a validation set of 381 patients with acute GVHD grade 1-4 at onset and treated with systemic steroids.

Preliminary analyses (not shown) determined that DO measurements predicted D28 non-response and D180 overall survival (OS). HLA match (match vs. mismatched; Odds Ratio (OR) 1.5, p=0.07), conditioning intensity (full vs. reduced; OR 1.7, p=0.04), and GVHD onset grade (grade 3-4 vs. grade 1-2; OR 2.2, p=0.001) predicted D28 non-response in univariate analysis, while age at transplant 55 years vs. <55 years), donor (unrelated vs. related), and stem cell source (peripheral blood vs. bone marrow/cord) did not.

After adjustment for the three clinical characteristics which predict D28 response, multivariate analysis of the 11 protein concentrations showed that three biomarkers predicted D28 response (ST2, p=0.001; IL1sRII, p=0.07; IL8, and p=0.03) and seven biomarkers predicted post-therapy 0180 OS (ST2, p=0.003; IL1sRII, p=0.07; IL8, p=0.05; Elafin, p=0.06; MIF, p=0.04; TNFR, p=0.03; and Reg3α, p=0.002 in gut-GVHD subset). Using logistic regression, the ability of the seven biomarkers, and ST2 alone, to predict for D28 non-responsiveness was examined, since ST2 was the most significant marker in all previous analyses.

A high biomarker value was defined as a plasma concentration greater than 50% above the median value of the responders group. A high panel was defined as having at least 5 of 7 high biomarkers. Patients with high ST2 levels (as measured by ELISA) were 2.6 times more likely not to respond to therapy independent of the aforementioned significant clinical characteristics (p<0.001) while patients with a high panel were only 1.9 times more likely not to respond (p=0.004). Thus, only ST2 measurement was used for further analyses.

Because ST2 concentrations correlated with response, it was hypothesized that ST2 would predict D180 non-relapse mortality (NRM) independent of GVHD onset grade, the strongest clinical predictor of NRM (20% for GVHD grade 1-2 vs. 50% for GVHD grade 3-4, Hazard ratio (HR) 3.0, p<0.001). D180 post-therapy NRM and HR showing the relationship between ST2 and GVHD onset are shown in Table 13. Patients with low ST2 had a similar NRM regardless of GVHD grade, indicating that ST2 provides important prognostic information at initiation of therapy above the GVHD grade stage.

TABLE 13
ST2 as a marker for the prediction of D 180 post-therapy NRM

| Onset GVHD | D 180 post- | Hazard Ratio | |
|---|---|---|---|
| grade % D 0 ST2 level | therapy NRM | (compared to (a) | p-value |
| (a) Low ST2 & | 11% | | |
| Grade 1-2 (N = 130) | | | |
| (b) Low ST2 & | 9% | 0.8 | 0.80 |
| Grade 3-4 (N = 22) | | | |
| (c) High ST2 & | 28% | 2.9 | <0.001 |
| Grade 1-2 (N = 165) | | | |
| (d) High ST2 & | 64% | 9.0 | <0.001 |
| Grade 3-4 (N = 64) | | | |

In conclusion, soluble ST2, the form measured by ELISA, is a decoy receptor that drives the Th2 phenotype toward Th1, a mechanism by which it may act in the pathophysiology of resistant GVHD. ST2 concentrations obtained at initiation of GVHD therapy significantly enhance the accuracy of outcome prediction independent of GVHD grade. Measurement of ST2 allows for early identification of patients at risk for subsequent non-response and mortality, and provides a promising target for novel therapeutic interventions.

### Example 8

### Prognostic Value of ST2 Concentrations in Subjects with GVHD

The clinical utility of any biomarker is greatly enhanced when it provides prognostic information regarding the future status of a disease and/or subject, e.g. the likelihood of response to treatment Early identification of patients who will not respond to GVHD therapy is extremely important because these patients are at high risk of death. Early identification will allow improved risk-stratification of patients presenting with signs of GVHD and may permit alternative testing or additional therapies before the development of refractory disease. Patients with high ST2 levels at therapy initiation for GVHD were 2.3 times more likely to be non-responders by day 28 of therapy [95% Confidence interval (CI): 1.5-2.6] and 3.6 (CI: 2.2-5.8) times more likely to be decreased by 6 months after therapy compared to those with low ST2, independent of GVHD grade which was the strongest predictor of response to treatment so far. A high ST2 level is defined as an ST2 concentration at therapy initiation of >740 pg/mL and a low ST2 level is defined as an ST2 concentration at therapy initiation ≦740 pg/mL.

### Example 9

### Predictive Value of ST2 Concentrations for Occurrence of GVHD and Mortality

The clinical utility of any biomarker is greatly enhanced when it provides predictive information regarding the disease before clinical signs are visible. The ability to identify patients with high ST2 concentrations early in their transplant course before GVHD development has therapeutic consequences including more stringent monitoring and potential preemptive interventions. Therefore, the value of ST2 plasma levels in human subjects taken at D14 post HCT predict (1) development of GVHD by D100 post-HCT, (2) D180 post-HCT non-relapse mortality (NRM), and (3) one year post-HCT overall survival. Because ST2 concentrations were different between conditioning intensities, three models for prediction were implemented using the median ST2 concentrations for chemotherapy-based full intensity conditioning, for reduced intensity conditioning, and for total body irradiation-based full intensity conditioning as cutpoints. The medians were chosen because GVHD status was unknown at the time of ST2 measurement.

In multivariate analysis including the clinical characteristics of age, disease status, donor source, and HLA match, high ST2 expression (i.e., high ST2 level) predicted the development of GVHD by D100 in patients receiving chemotherapy-based full intensity conditioning and total body irradiation-based full intensity conditioning (HR 1.5, CI: 1.1-2.0 and HR 2.0, CI: 0.9-4.3) independent of the clinical characteristics. In addition, when NRM was examined, patients with high ST2 level at D14 had increased risk of NRM at 6 months for all conditioning regimens (HR 2.8, CI: 1.6-4.8; HR 2.6, CI: 1.1-6.5; and HR 4.8, CI: 1.6-14.4) after adjustment for the clinical characteristics. High ST2 level was not associated with increased risk of relapse mortality 1 year after HCT. Thus, overall survival (OS) at 1 year was decreased in patients with high ST2 levels at D14. Under these circumstances, at about D14 post-HCT, a high or increased level of ST2 is defined as an ST2 concentration of greater than (>) about 600±200 pg/mL for patients who received chemotherapy-based full intensity conditioning, of greater than (>) about 300±100 pg/mL for patients who received reduced intensity conditioning, and of greater than (>) about 1660±500 pg/mL for patients who received total body irradiation-based full intensity conditioning.

### Example 10

### Use of a Four Biomarker Panel in Predicting GVHD after Transplant

The ability to identify patients at high risk for GVHD early in their transplantation regimen allows for preemptive interventions. To determine whether validated biomarkers can predict GVHD before the appearance of clinical symptoms, the expression level of four biomarkers (i.e., IL2R-α, TNFR1, elafin, and REG3α) in day 7 and 14 post-HSCT samples from 513 patients who underwent unrelated HSCT and had not yet developed GVHD were evaluated. Measurement of this biomarker panel pre-HSCT predicted grade II-IV GVHD with a specificity of 75% and sensitivity of 57%.

### Example 11

### Formulas for Predicting GVHD after Transplant

Acute GVHD is the primary limitation of HCT. Formulas were developed for predicting probability or risk of GVHD in a patient by calculating a score and then determining a probability from that score from data collected from a pool of over 800 patients.

The formulas comprise data from biomarker analysis along with various clinical parameters. In one embodiment, for example, a panel of four biomarkers from a biological sample of a patient are analyzed for protein level and data relating to several clinical observations or characteristics of the patient is also collected. Data from the biomarker analysis and collection of clinical parameters is factored into a formula for calculation of a score for each patient. Such clinical parameters and patient characteristics are patient age, type of transplantation (i.e., bone marrow versus peripheral blood stem cell), matching of HLA loci, whether patient received treatment with both tacrolimus and methotrexate, whether patient received a high toxicity conditioning regimen, and whether patient did or did not receive total body irradiation. High toxicity conditioning in a patient is an intense, myeloablative conditioning regimen prior to HCT aimed at reducing tumor burden. Total body irradiation (TBI) was considered to have been administered to the patient if the patient received a dose of TBI greater than 500 centigrade. If the dosage of radiation was less than 500 centigrade, the patient was considered to be without TBI.

A patient will receive a “score” equal to A+B, wherein “A” is computed from biomarker data and “B” is computed from clinical parameter data of the patient. Each patient's score is then converted to a predicted probability (p) of GVHD using the following formula:

$$p = \frac{{e}^{score}}{1 + {e}^{score}}$$

so that “p” will lie somewhere between 0 and 1. Each patient then gets a score based on the sum of the different factors as shown in the formulas below. Different formulas are used depending on whether the transplant was from a related donor or an unrelated donor.

More specifically, to compute “B” in the formula, protein concentrations (ng/ml) of four biomarkers, including REG3α, elafin, TNFR1, and IL2Rα, were measured in a biological sample from each patient one week after transplant. To compute “A” in the formula, the following clinical observations and/or patient characteristics/variables were recorded and were inputed in the formula:
Age=1 if patient's age >55 yo; age=0 if patient's age <=55 yo,
BM (bone marrow)=1 if bone marrow transplantation; BM=0 if peripheral blood transplantation;
Mismatch=1 if patient does not match all, i.e., eight of eight HLA loci, 2 genes for each of the four loci, HLA-A, B, C, and DR, with the transplant; mismatch=0 if patient matches all eight loci;
TM=1 if patient received both tacrolimus (Tacro) and methotrexate (MTX); TM=0 if patient did not receive both Tacro and MTX;
Tox1=1 if patient received high toxicity conditioning without total body irradiation (TBI); Tox1=0 if patient did not receive high toxicity conditioning without TBI; and
Tox2=1 if patient received high toxicity conditioning with TBI; Tox2=0 if patient does not receive high toxicity conditioning with TBI.

### A) Related Donor Transplants

A recipient of a related donor transplant will receive a “score” equal to A+B, wherein

A=−3.57+0.54×Age−16.83×BM+1.35×Mismatch−0.08×TM+0.35×Tox1+0.47×Tox2,

wherein the values of “0” or “1” are multiplied by a conversion factor to determine “A;” and wherein

B=0.37×log IL2Rα−0.06×log TNFR1−0.12×log Elafin−0.03×log Reg3α,

wherein the log base 2 of each biomarker protein level (ng/ml) is multiplied by a conversion factor to determine “B.”

Each patient's score is then converted to a predicted probability, p, of GVHD using the following formula:

$$p = \frac{{e}^{score}}{1 + {e}^{score}}$$

so that “p” will lie somewhere between 0 and 1.

For related donors, a patient is determined to have a positive test result, i.e., a positive test result for predicting GVHD, if their p value is above 0.38.

Two examples of such related donor transplant are set out below.

### Example 1

### Related Donor Transplant

A 45 year-old patient who did not develop a GVHD had the following characteristics:

Age=0; BM=0; Mismatch=0; TM=1; Tox1=0; Tox2=1

This patient's biomarker levels at day 7 post-transplant were converted using log base 2 of x, wherein x is the biomarker protein level in ng/ml for IL2rα, TNFR1, and elafin, and in pg/ml for Reg3α as follows:

IL2rα=2,961 (log IL2rα=11.53)

TNFR1=2,543 (log TNFR1=11.31)

Elafin=16,000 (log Elafin=17.29)

Reg3α=54 (log Reg3α=5.78)

Using the formulae above, A=−2.30 and B=1.27. The score is therefore −1.03 and results in a predicted probability (p) of p=0.26. Since this value of p is less than the threshold of 0.38, this patient would receive a negative test result.

### Example 2

### Related Donor Transplant

A 56-year old patient who developed GVHD at Day 22 had the following characteristics:

Age=1; BM=0; Mismatch=1; TM=1; Tox1=1; Tox2=0

This patient's biomarker levels at day 7 post-transplant were:

IL2rα=20,954 (log IL2rα=14.35)

TNFR1=5,864 (log TNFR1=12.52)

Elafin=8,408 (log Elafin=13.04)

Reg3α=58 (log Reg3α=5.86)

Using the formulae above, A=−2.75 and B=2.75. The score is therefore 0 and results in a p value of p=0.50: Since this value of p is greater than the threshold of 0.38, this patient would receive a positive test result.

### B) Unrelated Donor Transplants

A recipient of an unrelated donor transplant will receive a “score” equal to A+B, wherein

A=−1.87+0.16×Age+0.23×Match+−0.28×TM+0.18×Tox1+1.25×Tox2

wherein the values of “0” or “1” are multiplied by a conversion factor to determine “A;” and wherein

B=0.86×log IL2Rα−0.49×log TNFR1−0.23×log Elafin+0.06×log Reg3α

wherein the log base 2 of each biomarker protein level (ng/ml) is multiplied by a conversion factor to determine “B.”

The variables, explained in more detailed herein above, that were used to compute “A” are as follows:

Age=1 if age>55 yo & 0 if age 550
Match=1 if matched & 0 if mismatched
TM=1 if Tacro/MTX given & 0 if Tacro/MTX not given
Tox1=1 if given high toxicity conditioning without TBI & 0 otherwise
Tox2=1 if given high toxicity conditioning with TBI & 0 otherwise

Each patient's score was then converted to a predicted probability, p, of GVHD using the following formula:

$$p = \frac{{e}^{score}}{1 + {e}^{score}}$$

so that “p” will lie somewhere between 0 and 1.
A patient is then determined to have a positive test result, i.e., probability or risk of GVHD, if their value of p is above about 0.33.

Two examples of such unrelated donor transplants are set out below.

### Example 1

### Unrelated Donor Transplant

A 44 year-old patient who did not develop GVHD had the following characteristics:

### Age=0; Match=1; TM=1; Tox1=0; Tox2=0

This patient's biomarker levels at day 7 post-transplant were:
IL2rα=1,136 (log IL2rα=10.15)

### TNFR1=5,629 (log TINFR1=12.46)

### Elafin=10,434 (log Elafin=13.35)

### Reg3α=158 (log Reg3α=7.30)

Using the formulae above, A=−1.92 and B=0.14. The score is therefore −1.78 and results in a p value of p=0.14. Since this value of p is less than the threshold of 0.33, this patient would receive a negative test result. Thus, this patient would not have been predicted to develop GVHD and this patient did not.

### Example 2

### Unrelated Donor Transplant

A 63-year old patient who developed GVHD at Day 30 had the following characteristics:

### Age=1; Match=0; TM=0, Tox1=1; Tox2=0

This patient's biomarker levels at day 7 post-transplant were:
IL2rα=936 (log IL2rα=9.87)

### TNFR1=1,249 (log TNFR1=10.29)

### Elafin=1,918 (log Elafin=10.91)

### Reg3α=23 (log Reg3α=4.52)

Using the formulae above, A=−1.54 and B=1.34. The score is therefore −0.20 and results in a p value of p=0.45. Since this value of p is greater than the threshold of 0.33, this patient would receive a positive test result. Thus, this patient would have been predicted to develop GVHD and this patient did.

The disclosure has been described in terms of particular embodiments found or proposed to comprise specific modes for the practice of the disclosure. Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

1. A method for detecting graft-versus-host disease (GVHD) in a subject, the method comprising:
measuring a level of a biomarker in a biological sample isolated from the subject, wherein the biomarker is regenerating islet-derived 3-alpha (REG3α), and wherein an increased level of the biomarker present in the biological sample compared to a control level indicates GVHD in the subject.

2. A method for treating graft-versus-host disease (GVHD) in a subject suffering from GVHD or at risk of suffering from GVHD, the method comprising the steps of:
(a) identifying the subject at risk of suffering from GVHD,
(b) measuring a level of a biomarker in a biological sample isolated from the subject, wherein the biomarker is regenerating islet-derived 3-alpha (REG3α), and wherein an increased level of the biomarker present in the biological sample compared to a control level indicates GVHD in the subject, and
(c) administering an effective amount of a treatment for GVHD to the subject.

3. A method for determining efficacy of a treatment for GVHD in a subject suffering from graft-versus-host disease (GVHD), the method comprising the steps of:
(a) administering to the subject the treatment for GVHD, and
(b) measuring a level of biomarker in a biological sample obtained from the subject, wherein the biomarker is regenerating islet-derived 3-alpha (REG3α), and wherein a decrease in the level of the biomarker relative to the level of the biomarker prior to administration of the treatment, indicates that the treatment is effective for treating GVHD in the subject.

4. The method of claim 1 further comprising measuring a level of a second biomarker or a combination of biomarkers selected from the group consisting of: interleukin 2 receptor alpha (IL2Rα), tumor necrosis factor receptor superfamily member 1A (TNFRSF1A or TNFR1), interleukin 8 (IL-8), hepatocyte growth factor (HGF), and elafin in a biological sample, wherein an increased level of the biomarker present in the biological sample compared to a control level indicates GVHD in the subject.

5. The method of claim 3 further comprising measuring a level of a second biomarker or a combination of biomarkers selected from the group consisting of: interleukin 2 receptor alpha (IL2Rα), tumor necrosis factor receptor superfamily member 1A (TNFRSF1A or TNFR1), interleukin 8 (IL-8), hepatocyte growth factor (HGF), and elafin in a biological sample, wherein a decreased level of the biomarker present in the biological sample compared to a control level indicates that the treatment is effective for treating GVHD in the subject.

6. A method for predicting graft-versus-host disease (GVHD) in a subject, the method comprising:
measuring biomarker level for a combination of biomarkers in a biological sample isolated from the subject, wherein the combination of biomarkers comprises regenerating islet-derived 3-alpha (REG3α), IL2Rα, and elafin, and wherein an increased level of each of the biomarkers in the combination of biomarkers present in the biological sample compared to a control level of each biomarker predicts GVHD in the subject.

7-9. (canceled)

10. A method for predicting a subject's response to a treatment for graft-versus-host disease (GVHD), the method comprising:
measuring a level of a biomarker in a biological sample isolated from the subject, wherein the biomarker is ST2, and wherein an increased level of the biomarker present in the biological sample compared to a control level predicts lack of effectiveness of the treatment for GVHD in the subject.

11. A method for detecting effectiveness of a treatment for graft-versus-host disease (GVHD) in a subject undergoing treatment for GVHD, the method comprising:
measuring a level of a biomarker in a biological sample isolated from the subject, wherein the biomarker is ST2, and wherein an increased level of the biomarker present in the biological sample compared to a control level indicates lack of effectiveness of the treatment for GVHD in the subject.

12. A method for detecting graft-versus-host disease (GVHD) in a subject, the method comprising:
measuring a level of a biomarker in a biological sample isolated from the subject, wherein the biomarker is ST2, and wherein an increased level of the biomarker present in the biological sample compared to a control level indicates GVHD in the subject.

13. A method for treating graft-versus-host disease (GVHD) in a subject suffering from GVHD or at risk of suffering from GVHD, the method comprising the steps of:
(a) identifying the subject at risk of suffering from GVHD,
(b) measuring a level of a biomarker in a biological sample isolated from the subject, wherein the biomarker is ST2, and wherein an increased level of the biomarker present in the biological sample compared to a control level indicates GVHD in the subject, and
(c) administering an effective amount of a treatment for GVHD to the subject.

14. A method for determining efficacy of a treatment for graft-versus-host disease (GVHD) in a subject suffering from GVHD, the method comprising the steps of:
(a) administering to the subject the treatment for GVHD, and
(b) measuring a level of biomarker in a biological sample obtained from the subject, wherein the biomarker is ST2, and wherein a decrease in the level of the biomarker relative to the level of the biomarker prior to administration of the treatment, indicates that the treatment is effective for treating GVHD in the subject.

15-17. (canceled)

18. A method for treating graft-versus-host disease (GVHD) in a subject at risk of suffering from GVHD, the method comprising the steps of:
(a) identifying the subject at risk of suffering from GVHD by measuring a level of a biomarker or a combination of biomarkers in a biological sample isolated from the subject, wherein the biomarker is REG3α or ST2, and wherein an increased level of the biomarker present in the biological sample compared to a control level indicates GVHD or risk of GVHD in the subject, and
(b) administering an effective amount of a treatment for GVHD to the subject at risk of suffering from GVHD.

19-27. (canceled)

28. A kit comprising reagents for measuring the biomarker or combination of biomarkers according to the method of claim 1, wherein the biomarker or combination of biomarkers is present in a biological sample isolated from the subject.

29. A kit for assessing susceptibility of developing graft-versus-host disease (GVHD) in a subject, the kit comprising reagents for selectively detecting a level of a biomarker or a combination of biomarkers in a biological sample from a subject, wherein the biomarker or the combination of biomarkers is selected from the group consisting of regenerating islet-derived 3-alpha (REG3α), ST2, or a combination of REG3α and ST2.

30. The kit of claim 28, wherein the biomarker or the combination of biomarkers further comprises a biomarker or combination of biomarkers selected from the group consisting of elafin, tumor necrosis factor receptor 1 (TNFR1), interleukin-2 receptor alpha chain (IL2Rα), interleukin 8 (IL-8), and hepatocyte growth factor (HGF).

31-35. (canceled)

36. A method of determining susceptibility of developing graft-versus-host disease (GVHD) in a subject, the method comprising:
a) analyzing a biological sample from the subject to obtain level of a biomarker or a combination of biomarkers in a subject, wherein the biomarker or the combination of biomarkers is selected from the group consisting of regenerating islet-derived 3-alpha (REG3α), ST2, and REG3α and ST2; and
b) assessing a clinical parameter or a combination of clinical parameters in the subject, wherein the presence of an elevated level of the biomarker or combination of biomarkers and the presence of a clinical parameter or a combination of clinical parameters associated with increased risk of GVHD indicates that the subject is susceptible of developing GVHD.

37. A method of determining susceptibility of developing graft-versus-host disease (GVHD) in a subject, the method comprising:
a) analyzing a biological sample from the subject to obtain level of a biomarker or a combination of biomarkers in a subject, wherein the biomarker or the combination of biomarkers is selected from the group consisting of regenerating islet-derived 3-alpha (REG3α), ST2, and REG3α and ST2; and
b) calculating a risk score or probability as an indicator of the subject's susceptibility of developing GVHD based upon level of the biomarker or the combination of biomarkers.

38. A method of determining susceptibility of developing graft-versus-host disease (GVHD) in a subject, the method comprising:
a) analyzing a biological sample from the subject to obtain level of a biomarker or a combination of biomarkers in a subject, wherein the biomarker or the combination of biomarkers is selected from the group consisting of regenerating islet-derived 3-alpha (REG3α), ST2, and REG3α and ST2;
b) assessing a clinical parameter or a combination of clinical parameters in the subject; and
c) calculating a risk score or probability as an indicator of the subject's susceptibility of developing GVHD based upon level of the biomarker or the combination of biomarkers and the clinical parameter or the combination of clinical parameters.

39. The method of claim 36, wherein the biomarker or the combination of biomarkers further comprises a biomarker or combination of biomarkers selected from the group consisting of elafin, tumor necrosis factor receptor 1 (TNFR1), interleukin-2 receptor alpha chain (IL2Rα), interleukin 8 (IL-8), and hepatocyte growth factor (HGF).

40. (canceled)

41. The method of claim 36, wherein the combination of biomarkers comprises REG3α, elafin, TNFR1, and IL2Rα.

42-58. (canceled)

59. A system for identifying susceptibility of developing graft-versus-host disease (GVHD) in a subject, the system comprising:
at least one processor; at least one computer-readable medium;
a susceptibility database operatively coupled to a computer-readable medium of the system and containing population information correlating level of a biomarker or a combination of biomarkers in a subject to susceptibility to developing GVHD in a population of humans, wherein the biomarker or the combination of biomarkers is selected from the group consisting of regenerating islet-derived 3-alpha (REG3α), ST2, and REG3α and ST2;
a measurement tool that receives an input about the subject and generates information from the input about the level of the biomarker or the combination of biomarkers in the subject, wherein an elevated level of the biomarker or the combination of biomarkers is associated with increased susceptibility to GVHD;
and an analysis tool that is operatively coupled to the susceptibility database and the measurement tool is stored on a computer-readable medium of the system, is adapted to be executed on a processor of the system, to compare the information about the subject with the population information in the susceptibility database and generate a conclusion with respect to susceptibility of developing GVHD for the subject.

60. The system according to claim 59, wherein the susceptibility database further comprises population information correlating a clinical parameter or a combination of clinical parameters in the subject to susceptibility to developing GVHD in a population of humans to susceptibility to developing GVHD in a population of humans; and wherein the measurement tool further generates information from the input about the clinical parameter or combination of clinical parameters in the subject, and the impact of the presence or absence of the clinical parameter or combination of clinical parameters on identifying susceptibility of developing GVHD.

61-76. (canceled)

77. A regimen for treating graft-versus-host disease (GVHD) in a subject, the regimen comprising:
a) measuring a biomarker or a combination of biomarkers in a biological sample from a subject with GVHD or at risk of GVHD, wherein the biomarker or the combination of biomarkers is selected from the group consisting of regenerating islet-derived 3-alpha (REG3α), ST2, and REG3α and ST2;
b) wherein an increased level of the biomarker or combination of biomarkers compared with control indicates that the subject is suffering from GVHD or is at risk of GVHD; and
c) for a subject with GVHD or a risk, probability, or susceptibility of developing GVHD based upon level of the biomarker or the combination of biomarkers and presence or absence of the clinical parameter or the combination of clinical parameters, prescribing or administering a treatment regimen that includes a steroid, an immunosuppressant, or a combination of steroid and immunosuppressant.

78. A regimen for treating graft-versus-host disease (GVHD) in a subject, the treatment regimen comprising:
a) measuring a biomarker or a combination of biomarkers in a biological sample from a subject at risk of GVHD, wherein the biomarker or the combination of biomarkers is selected from the group consisting of regenerating islet-derived 3-alpha (REG3α), ST2, and REG3α and ST2;
b) assessing a clinical parameter or a combination of clinical parameters in the subject; and
c) for a subject with a risk, probability, or susceptibility of developing GVHD based upon level of the biomarker or the combination of biomarkers and presence or absence of the clinical parameter or the combination of clinical parameters, prescribing or administering a treatment regimen that includes a steroid, an immunosuppressant, or a combination of steroid and immunosuppressant.

79-90. (canceled)

91. Use of measurement of an elevated level of a biomarker or a combination of biomarkers in a biological sample from a subject at risk of graft-versus-host disease (GVHD) compared to control level, wherein the biomarker or the combination of biomarkers is selected from the group consisting of regenerating islet-derived 3-alpha (REG3α), ST2, and REG3α and ST2, for the selection of a treatment regimen for the subject.

92-104. (canceled)

105. A method of decreasing toxicity of a regimen for treating graft-versus-host disease (GVHD) in a subject diagnosed with GVHD, wherein the subject is being treated with a more aggressive therapy for GVHD comprising:
a) measuring a level of a biomarker or a combination of biomarkers in a biological sample from the subject diagnosed with GVHD, wherein the biomarker or the combination of biomarkers is selected from the group consisting of regenerating islet-derived 3-alpha (REG3α), ST2, and REG3α and ST2; and wherein a decreased level of the biomarker or combination of biomarkers compared with control level indicates that the subject is at reduced risk of GVHD; and
b) prescribing or administering to the subject a less aggressive therapy or regimen for treating GVHD.

106. The method of claim 105, wherein the biomarker or the combination of biomarkers further comprises a biomarker or combination of biomarkers selected from the group consisting of elafin, tumor necrosis factor receptor 1 (TNFR1), interleukin-2 receptor alpha chain (IL2Rα), interleukin 8 (IL-8), and hepatocyte growth factor (HGF).

107-109. (canceled)
